SEQUENCE LISTING (19 sequences)

SEQ ID NO: 1 - DNA, 954 bp, Homo sapiens
atgacgtcacggactcgggtcacatggccaagtccgccccgccccctccccgtccccgccgctgcagcggtcgccttcggagcgaagggtaccgacccggcagaagctcggagctctcggggtatcgaggaggcaggcccgcgggcgcacgggcgagcgggccgggagccggagcggcggaggagccggcagcagcggcgcggcgggctccaggcgaggcggtcgacgctcctgaaaacttgcgcgcgcgctcgcgccactgcgcccggagcgatgaagatggtcgcgccctggacgcggttctactccaacagctgctgcttgtgctgccatgtccgcaccggcaccatcctgctcggcgtctggtatctgatcatcaatgctgtggtactgttgattttattgagtgccctggctgatccggatcagtataacttttcaagttctgaactgggaggtgactttgagttcatggatgatgccaacatgtgcattgccattgcgatttctcttctcatgatcctgatatgtgctatggctacttacggagcgtacaagcaacgcgcagcctggatcatcccattcttctgttaccagatctttgactttgccctgaacatgttggttgcaatcactgtgcttatttatccaaactccattcaggaatacatacggcaactgcctcctaattttccctacagagatgatgtcatgtcagtgaatcctacctgtttggtccttattattcttctgtttattagcattatcttgacttttaagggttacttgattagctgtgtttggaactgctaccgatacatcaatggtaggaactcctctgatgtcctggtttatgttaccagcaatgacactacggtgctgctacccccgtatgatgatgccactgtgaatggtgctgccaaggagccaccgccaccttacgtgtctgcctaa

SEQ ID NO: 2 - protein, 317 aa, Homo sapiens
MTSRTRVTWPSPPRPLPVPAAAAVAFGAKGTDPAEARSSRGIEEAGPRAHGRAGREPERRRSRQQRRGGLQARRSTLLKTCARARATAPGAMKMVAPWTRFYSNSCCLCCHVRTGTILLGVWYLIINAVVLLILLSALADPDQYNFSSSELGGDFEFMDDANMCIAIAISLLMILICAMATYGAYKQRAAWIIPFFCYQIFDFALNMLVAITVLIYPNSIQEYIRQLPPNFPYRDDVMSVNPTCLVLIILLFISIILTFKGYLISCVWNCYRYINGRNSSDVLVYVTSNDTTVLLPPYDDATVNGAAKEPPPPYVSA

SEQ ID NO: 3 - DNA, 738 bp, Homo sapiens
atggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaa

SEQ ID NO: 4 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 5 - RNA, 25 nt, Artificial Sequence
  Description of Artificial Sequence Synthetic oligonucleotide
cccguaugaugaugccacugugaau

SEQ ID NO: 6 - RNA, 25 nt, Artificial Sequence
  Description of Artificial Sequence Synthetic oligonucleotide
gggcauacuacuacggugacacuua

SEQ ID NO: 7 - RNA, 25 nt, Artificial Sequence
  Description of Artificial Sequence Synthetic oligonucleotide
ucggcgucugguaucugaucaucaa

SEQ ID NO: 8 - RNA, 25 nt, Artificial Sequence
  Description of Artificial Sequence Synthetic oligonucleotide
agccgcagaccauagacuaguaguu

SEQ ID NO: 9 - RNA, 25 nt, Artificial Sequence
  Description of Artificial Sequence Synthetic oligonucleotide
caucaaugguaggaacuccucugau

SEQ ID NO: 10 - RNA, 25 nt, Artificial Sequence
  Description of Artificial Sequence Synthetic oligonucleotide
guaguuaccauccuugaggagacua

SEQ ID NO: 11 - DNA, 50 bp, Artificial Sequence
  Description of Artificial Sequence Synthetic oligonucleotide
caccgctgatccggatcagtataaccgaagttatactgatccggatcagc

SEQ ID NO: 12 - DNA, 50 bp, Artificial Sequence
  Description of Artificial Sequence Synthetic oligonucleotide
aaaagctgatccggatcagtataacttcggttatactgatccggatcagc

SEQ ID NO: 13 - RNA, 25 nt, Artificial Sequence
  Description of Artificial Sequence Synthetic oligonucleotide
acagcacgcuaauaaugcaauuacu

SEQ ID NO: 14 - RNA, 25 nt, Artificial Sequence
  Description of Artificial Sequence Synthetic oligonucleotide
ugucgugcgauuauuacguuaauga

SEQ ID NO: 15 - RNA, 25 nt, Artificial Sequence
  Description of Artificial Sequence Synthetic oligonucleotide
gagagacaacuugacauuguggaca

SEQ ID NO: 16 - RNA, 25 nt, Artificial Sequence
  Description of Artificial Sequence Synthetic oligonucleotide
cucucuguugaacuguaacaccugu

SEQ ID NO: 17 - RNA, 25 nt, Artificial Sequence
  Description of Artificial Sequence Synthetic oligonucleotide
ggauacccaaggagacgaagcugaa

SEQ ID NO: 18 - RNA, 25 nt, Artificial Sequence
  Description of Artificial Sequence Synthetic oligonucleotide
ccuauggguuccucugcuucgacuu

SEQ ID NO: 19 - protein, 6 aa, Artificial Sequence
  Description of Artificial Sequence Synthetic 6xHis tag
HHHHHH

(19) United States
(12) Patent Application Publication
(10) Pub. No.: US 2012/0052079 A1
(43) Pub. Date: Mar. 1, 2012

(54) Compositions, Kits, and Methods for Predicting Anti-Cancer Response to Anthracyclines

(75) Inventors: Andrea L. Richardson, Chestnut Hill, MA (US); Zhigang C. Wang, Chestnut Hill, MA (US)

(73) Assignee: Dana-Farber Cancer Institute, Inc., Boston, MA (US)

(21) Appl. No.: 13/206,829

(22) Filed: Aug. 10, 2011

Related U.S. Application Data
(60) Provisional application No. 61/401,251, filed on Aug. 10, 2010.

(51) Int. Cl. A61K 39/395; C40B 30/04; G01N 33/566; A61P 35/00; A61K 31/704; C07H 21/00; C07K 16/18; C07H 21/02; C12Q 1/68; A61K 31/713

(52) U.S. Cl. 424/174.1; 435/6.11; 435/6.12; 506/9; 435/7.9; 424/178.1; 514/44 A; 514/34; 536/24.3; 530/389.7; 536/24.5

(57) ABSTRACT

The present invention is based, in part, on the discovery that amplification of human chromosome 8q22-23 regions and over-expression of 8q22-23 genes (e.g., LAPTM4B and YWHAZ) is associated with and predictive of resistance to anthracycline-type chemotherapy. Accordingly, the invention relates to compositions, kits, and methods for predicting the response of cancer cells, e.g., breast, prostate, lung, ovarian, pancreatic, liver, and colon malignancies to anthracyclines.

### RELATED APPLICATIONS

This application claims the benefit U.S. provisional application No. 61/401,251, filed Aug. 10, 2010; the content of said application is incorporated herein in its entirety by this reference.

### GOVERNMENT FUNDING

This invention was made with government support under Grant NIH RO1 CA89393 awarded by the National Institutes of Health and under Concept Award Grant BC053041 awarded by the Department of Defense. The U.S. government has certain rights in the invention. This statement is included solely to comply with 37 C.F.R. §401.14(a)(f)(4) and should not be taken as an assertion or admission that the application discloses and/or claims only one invention.

### BACKGROUND OF THE INVENTION

Cancer subjects are often administered adjuvant chemotherapy after surgical removal of primary tumors in order to eradicate residual tumor cells. Residual tumor cells resistant to chemotherapy have a survival advantage in treated subjects that can accelerate metastatic disease. For example, breast cancer is the most common non-skin cancer of women in the United States, yet about 50% of subjects respond poorly, have disease progression, or relapse due to chemoresistance when treated with anthracycline-based adjuvant chemotherapy.

De novo resistance (i.e., pre-existing resistance prior to any exposure to anti-cancer therapies such as chemotherapeutic drugs) mechanisms within tumor cells before treatment are key factors leading to the failure of chemotherapeutic drugs to prevent metastatic recurrence. Although a number of multidrug resistance genes contributing to chemoresistance to specific drugs have been discovered, their overexpression is often induced during drug treatment (Dowsett et al. (2007) Breast Cancer Res. 9, R81; Gottesman et al. (2002) Nat. Rev. Cancer 2, 48-58; Turton et al. (2001) Oncogene 20, 1300-1306), and the expression of these genes in tumors before treatment is not generally useful for initial guidance of drug selection. Gene signatures generated from responses of tumor cell lines to drugs are reported to predict drug response in subjects (Potti et al. (2006) Nat. Med. 12, 1294-1300; Gyorffy et al. (2005) Oncogene 24, 7542-7551; Lee et al. (2007) Proc. Natl. Acad. Sci. USA 104, 13086-13091); however, such cell line-derived signatures have often not been predictive of response in clinical cases (Liedtke et al. (2010) Breast Cancer Res. Treat. 121, 301-309). Overall, oncological clinical interventions have been severely hampered by the fact that often only a percentage of subjects will respond favorably to a particular anti-cancer treatment. Medical oncologists currently cannot predict which subjects will or will not respond to the proposed chemotherapeutic treatment. Accordingly, there is a great need in the art to define predictive biomarkers of response to particular anti-cancer therapies.

### SUMMARY OF THE INVENTION

The present invention is based, at least in part, on the discovery that amplification of human chromosome 8q22-23 regions and over-expression of 8q22-23 genes (e.g., LAPTM4B and YWHAZ) is associated with and predictive of anti-cancer responses to anthracycline-type chemotherapy. Accordingly, in one aspect, the present invention features a method of predicting the outcome of treatment of a subject with an anthracycline, wherein the subject has a cell hyperproliferative disorder, comprising obtaining a biological sample from the subject, comparing the copy number of a marker in the sample to a control copy number of the marker, wherein said marker comprises region 98,778K to 101,970K on human chromosome 8 or a fragment thereof, and determining therefrom the outcome of treatment of the subject with an anthracycline. The present invention further features a method of predicting the outcome of treatment of a subject with an anthracycline wherein the subject has a hyperproliferative disorder, comprising obtaining a biological sample from the subject, and comparing: a) the amount, structure, subcellular localization, and/or activity of at least one marker in a subject sample, wherein the marker is selected from the group consisting of LAPTM4B, YWHAZ, and functionally similar homologs thereof; and b) the amount, structure, subcellular localization, and/or activity of the at least one marker in a control, wherein a significant difference in the amount, structure, subcellular localization, and/or activity of the at least one marker in the sample and the amount, structure, subcellular localization, and/or activity in the control is predictive of the outcome of treatment of the subject with an anthracycline.

In one aspect, the method further comprises determining a suitable treatment regimen for the subject. In one embodiment, the suitable treatment regimen comprises treatment with an anthracycline when the copy number of the marker is equal to or less than the control copy number of the marker or does not comprise treatment with an anthracycline when the copy number of the marker is greater than the control copy number of the marker. In another embodiment, the control copy number of the marker is the wild type copy number of the marker in the species to which the subject belongs.

In another aspect, the marker is LAPTM4B and YWHAZ.

In still another aspect, the control is determined from a non cell hyperproliferative disorder cell sample from the subject or member of the same species to which the subject belongs, such as a normalized amount, subcellular localization, structure, and/or activity in a cell or tissue of interest. In one embodiment, the control amount, subcellular localization, structure, and/or activity is the wild type amount, subcellular localization, structure, and/or activity, such as a normalized amount, subcellular localization, structure, and/or activity in a cell or tissue of interest, in the species to which the subject belongs.

In yet another aspect, the subject sample is obtained before the subject has received adjuvant chemotherapy. Alternatively, the subject sample is obtained after the subject has received adjuvant chemotherapy.

In another aspect, the sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow.

In still another aspect, the cell hyperproliferative disorder is selected from the group consisting of breast cancer, ovarian cancer, transitional cell bladder cancer, bronchogenic lung cancer, thyroid cancer, pancreatic cancer, prostate cancer, uterine cancer, testicular cancer, gastric cancer, soft tissue and osteogenic sarcomas, neuroblastoma, Wilms' tumor, malignant lymphoma (Hodgkin's and non-Hodgkin's), acute myeloblastic leukemia, acute lymphoblastic leukemia, Kaposi's sarcoma, Ewing's tumor, refractory multiple myeloma, and squamous cell carcinomas of the head, neck, cervix, and vagina.

In yet another aspect, the amount of the marker is determined by determining the level of expression or copy number of the marker. In one embodiment, the copy number is determined by using at least one technique selected from the group consisting of fluorescence in situ hybridization (FISH), quantitative PCR (qPCR), comparative genomic hybridization (CGH), and single-nucleotide polymorphism (SNP) array. In another embodiment, the level of expression of the marker in the sample is assessed by detecting the presence in the sample of a protein corresponding to the marker. In still another embodiment, the protein is detected using a reagent selected from the group consisting of an antibody, an antibody derivative, and an antibody fragment. In yet another embodiment, the level of expression of the marker in the sample is assessed by detecting the presence in the sample of a transcribed polynucleotide or portion thereof, wherein the transcribed polynucleotide (e.g., mRNA or a cDNA) comprises the marker. In another embodiment, determining the level of expression of the marker comprises the use of at least one technique selected from the group consisting of Northern blot analysis, reverse transcriptase PCR, real-time PCR, RNAse protection, and microarray analysis. In still another embodiment, the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

In another aspect, the significant difference is an increase in the amount, structure, subcellular localization, and/or activity of the subject sample relative to the control, indicating a reduced likelihood of efficacy of anthracycline treatment of the subject. Alternatively, the significant difference is a decrease in the amount, structure, subcellular localization, and/or activity of the subject sample relative to the control, indicating an increased likelihood of efficacy of anthracycline treatment of the subject. In one embodiment, the outcome of treatment is measured by at least one criteria selected from the group consisting of survival until mortality, pathological complete response, clinical complete remission, clinical partial remission, clinical stable disease, recurrence-free survival, metastasis free survival, and disease free survival.

In still another aspect, the anthracycline is selected from the group consisting of mitoxantrone, doxorubicin, aclarubicin, daunorubicin, epirubicin, idarubicin and combinations thereof.

The present invention further features a method of treating a subject afflicted with cancer comprising administering to the subject an agent which changes the subcellular localization of or modulates the amount and/or activity of a gene or protein corresponding to at least one marker, wherein the at least one marker is selected from the group consisting of LAPTM4B, YWHAZ, and functionally similar homologs thereof.

In one aspect, the method further comprises administering to the subject an anthracycline. In one embodiment, the anthracycline is selected from the group consisting of mitoxantrone, doxorubicin, aclarubicin, daunorubicin, epirubicin, idarubicin, and combinations thereof.

In another aspect, the marker is LAPTM4B and YWHAZ.

In still another aspect, the agent is administered in a pharmaceutically acceptable formulation.

In yet another aspect, the agent is an antibody or an antigen binding fragment thereof, which specifically binds to a protein corresponding to said marker. In one embodiment, the antibody is conjugated to a toxin or a chemotherapeutic agent.

In another aspect, the compound is an RNA interfering agent which inhibits expression of a gene corresponding to said marker. In one embodiment, the RNA interfering agent is an siRNA molecule or an shRNA molecule.

In still another aspect, the compound is an antisense oligonucleotide complementary to a gene corresponding to said marker.

In yet another aspect, the compound is a peptide or peptidomimetic.

In another aspect, the compound is a small molecule which inhibits activity of said marker. In one embodiment, the small molecule inhibits a protein-protein interaction between a marker and a target protein.

In still another aspect, the compound is an aptamer which inhibits expression or activity of said marker.

The present invention further features several kits. In one aspect, a kit is featured for predicting the outcome of treatment of a subject with a cell hyperproliferative disorder with an anthracycline, comprising a reagent for assessing the copy number of at least one marker, wherein the at least one marker comprises region 98,778K to 101,970K on human chromosome 8 or a fragment thereof. In another aspect, a kit is featured for assessing the outcome of treatment of a subject with a cell hyperproliferative disorder with an anthracycline, comprising a reagent for assessing the amount, structure, subcellular localization, and/or activity of at least one marker, wherein the at least one marker is selected from the group consisting of LAPTM4B, YWHAZ, and functionally similar homologs thereof. In one embodiment, the at least one marker is LAPTM4B and YWHAZ. In another embodiment, the reagent is selected from the group consisting of a nucleic acid molecule that hybridizes with the at least one marker. In still another embodiment, the reagent is selected from the group consisting of an antibody, and antibody derivative, and an antibody fragment.

In still another aspect, a kit is featured for treating a subject afflicted with cancer comprising an agent which changes the subcellular localization of or modulates the amount and/or activity of a gene or protein corresponding to at least one marker, wherein the at least one marker is selected from the group consisting of LAPTM4B, YWHAZ, and functionally similar homologs thereof. In one embodiment, the at least one marker is LAPTM4B and YWHAZ. In another embodiment, the kit further comprises an anthracycline. In still another embodiment, the anthracycline is selected from the group consisting of mitoxantrone, doxorubicin, aclarubicin, daunorubicin, epirubicin, idarubicin, and combinations thereof.

### BRIEF DESCRIPTION OF THE DRAWINGS

FIGS. 1A-1G show the relationship between 8q22 amplification, gene expression and cancer recurrence. FIG. 1A shows a PAM score. Genes with significant PAM scores are displayed according to their annotated location on chromosome 8. FIG. 1B shows metaphase FISH in normal human lymphoid cells using 8q22 probe RP11-347C18 (orange). FIG. 1C shows interphase FISH with 8q22 probe RP11-347C18 (orange), chromosome 8 centromere probe (green) and DAPI nuclear stain (blue) in a human tumor sample. FIG. 1D shows the correlation between 8q22 copy number detected by FISH and the 8qEI (regression R=0.65). FIG. 1E shows Kaplan-Meier analysis for distant recurrence-free survival (DRFS) of 85 Boston cases with or without 8q22 amplification identified by FISH (Cox hazard ratio=7.77). FIG. 1F shows DRFS for 52 Boston cases treated with doxorubicin and cyclophosphamide, according to 8q22 amplification identified by FISH (Cox hazard ratio=7.66). FIG. 1G shows disease-free survival (DFS) for 361 subjects from six independent cohorts who received adjuvant chemotherapy, according to 8qEI high and low based on 8qEI levels above or below median level in each of the six cohorts (hazard ratio=1.9).

FIG. 2 shows frequent copy gain in breast cancer. The fraction of cases with SNP copy number gain ≧4 was plotted along the human genome. Chromosome number 1 to the X chromosome is indicated from left to right as labeled across the top.

FIGS. 3A-3D show Kaplan-Meier analysis for outcomes according to 8q22 genomic gain or gene expression. Kaplan-Meier curves for distant recurrence-free survival (DRFS) in ER− cases from Boston (FIG. 3A) and disease-free survival (DFS) for cases without adjuvant systemic therapy from six combined cohorts (FIG. 3B) are shown, according to averaged expression of twelve 8q22 genes (8qEI). FIG. 3C and FIG. 3D show DFS of cases from six combined cohorts according to averaged LAPTM4B+YWHAZ expression (L+Y) in those who have received adjuvant chemotherapy (FIG. 3C) and those who have not received any adjuvant systemic therapy (FIG. 3D). Cox Hazard Ratio (HR) and P-value derived using Mantel-Cox log rank test are indicated in each panel.

FIG. 4 shows qRT-PCR measurements of relative mRNA levels in BT549 cells after siRNA knockdown. The blue, red, and white bars indicate the linear fraction of mRNA levels after knockdown by test siRNA oligonucleotides relative to a control siRNA oligonucleotide. Genes are indicated along the X-axis.

FIGS. 5A-5D show the results of knockdown of 8q22 genes by siRNA in tumor cell lines to determine the effect on sensitivity to anthracycline chemotherapy. FIG. 5A shows siRNA-mediated knockdown of the twelve 8q22 genes, described in Table 1, in BT549 breast tumor cells. Bars indicate the ratio of IC50 for the indicated drugs in gene-specific siRNA-treated cells, relative to control siRNA-treated cells. Percentage reduction in IC50 is indicated as follows: ♦♦, 63% reduction; ♦♦♦, 82%; ♦♦♦♦, >85%. Error bars represent means±s.d. of triplicate measurements. FIG. 5B shows relative mRNA levels for LAPTM4B and YWHAZ in the three breast cancer cell lines, MDA-MB-231 (MDA231), HCC38 and CAMA-1, after treatment with the indicated control or specific siRNAs. Levels are relative to HMEC reference sample. Error bars represent means±s.d. of triplicate measurements. FIG. 5C shows drug concentration-dependent cell survival curves for doxorubicin (top), cisplatin (middle), and paclitaxel (bottom) in cells transfected with control siRNA or the indicated gene-specific siRNAs. The approximate percentage reduction in IC50 is indicated as follows: ♦♦♦, 85%; ♦♦, 75%; and ♦, 50%. FIG. 5D shows Western blot results for pro-caspase-3 (pr-cas3), active caspase-3 (ac-cas3) and cleaved lower molecular weight PARP (cl-PARP), in MDA-MB-231 (MDA231) and BT549 cells transfected with siRNA specific for LAPTM4B (L), YWHAZ (Y) or scramble control (C), as indicated across the top. Cells were treated with carrier (no marker) or with doxorubicin or cisplatin (+) for 48 h, as indicated above each lane.

FIGS. 6A-6B show the correlation between relative LAPTM4B or YWHAZ mRNA levels and doxorubicin resistance in 16 breast cancer cell lines. The fifty percent inhibitory concentration (IC50) for each cell line is plotted on the y-axes and the relative transcripts level (log2) of LAPTM4B (FIG. 6A) and YWHAZ (FIG. 6B) are indicated on the x-axes.

FIGS. 7A-7G show LAPTM4B expression, intracellular doxorubicin distribution and drug-induced DNA damage in breast cancer cell lines. FIG. 7A shows mRNA levels of LAPTM4B and YWHAZ relative to a reference sample (lefty axis) and doxorubicin IC₅₀ concentration (righty axis) in three breast cancer cell lines, as indicated at the bottom. FIG. 7B and FIG. 7C show merged fluorescence analysis for doxorubicin (drug autofluorescence, red) and nuclear staining (DAPI, blue). Doxorubicin localized in the nucleus appears purple. Scale bars, 10 p.m. In particular, FIG. 7B shows intracellular doxorubicin distribution after 24 h of drug exposure in three breast tumor cell lines, BT549, MDA-MB-231 (MDA231) and SKBR3. FIG. 7C shows intracellular doxorubicin distribution in MDA-MB-231 cells transfected with control siRNA (top row) and LAPTM4B-specific siRNA (bottom row). Time points during drug exposure and after removal of drug from culture medium are shown above the images. FIG. 7D shows results of a Western blot analysis with phospho-H2AX-specific antibody and β-actin-specific control antibody in lysates of MDA-MB-231 cells transfected with the indicated control and gene-specific siRNA oligonucleotides at the indicated time points of drug exposure and after removal of drug from culture medium. FIG. 7E shows a plot of inhibition of cell growth by doxorubicin, paclitaxel and cisplatin in HMEC cells transfected with vectors containing GFP control, LAPTM4B-HA, or YWHAZ-HA. The percentages of viable cells, compared to transfected cells without drug treatment, are indicated on they axis (mean of triplicates±s.d.). ♦♦ indicates a >250% increase in IC50. FIG. 7F shows results of a Western blot analysis of HMEC cells transfected with vectors containing LacZ control, LAPTM4B-HA and YWHAZ-HA using HA tag-specific (anti-HA) antibody and β-actin-specific antibody. FIG. 7G shows intracellular doxorubicin distribution in HMEC cells transfected with LacZ control vector or with LAPTM4B-HA vector after 16 h of doxorubicin exposure. Merged fluorescence analysis for doxorubicin (drug autofluorescence, red) and nuclear staining (DAPI, blue). Doxorubicin localized in the nucleus appears purple. Scale bars, 10 μm.

FIGS. 8A-8F show LAPTM4B and YWHAZ expression and pCR to neoadjuvant chemotherapy. Cases were ranked according to mean sum expression of LAPTM4B (L) and YWHAZ (Y) (FIG. 8A-8C) and ROC plots for the performance of the LAPTM4B and YWHAZ genes to predict pCR (FIG. 8D-8F) for epirubicin in the whole breast cancer cohort (pCR, n=17; no pCR, n=101; FIGS. 8A and 8D), epirubicin in the ER−HER2− subcohort (pCR, n=9; no pCR, n=78; FIGS. 8B and 8E), and cisplatin in ER−PR-HER2− individuals with breast cancer (pCR, n=4; no pCR, n=20; FIGS. 8C and 8F). FIGS. 8D-8F show ROC plots, in which the solid diagonal lines indicate the performance of a random predictor, and the dashed line indicates the performance of the mean LAPTM4B and YWHAZ levels to predict pCR.

FIGS. 9A-9B show intracellular localization of LAPTM4B and doxorubicin distribution in mammary epithelial cells. FIG. 9A shows the results of merged immunofluorescence analyses for LAPTM4B (anti-His, FITC, green) localization, lysosomes (LysoTracker DND-99, red) and nuclear staining (DAPI, blue) in HMEC cells transfected with His-tagged LAPTM4B. FIG. 9B shows the results of merged fluorescence analyses by confocal microscopy for lysosomes (LysoTracker, green), doxorubicin (drug autofluorescence, red), and nuclear staining (DAPI, blue) in BT549 cells after 48 hours of drug treatment.

FIGS. 10A-10C show lysosome membrane permeabilization triggered by suppression of LAPTM4B expression. Merged fluorescence analysis for dextran (FITC, green) and nuclear staining (DAPI, blue) to show intracellular distribution of 4 kDa (top panels), 40 kDa (middle panels), or 70 kDa (lower panels) dextran particles in BT549 cells transfected with a scrambled control siRNA (FIG. 10A) and BT549 cells transfected with LAPTM4B-specific siRNA (FIG. 10B). FIG. 10C shows the results of mMerged fluorescence analyses for doxorubicin (drug autofluorescence, red), lysosomes (Lysotracker DND-26, green) and nuclear staining (DAPI, blue) in BT549 cells transfected with control siRNA (Ctrl, upper panels) and with LAPTM4B-specific siRNA (siRNA, lower panels) after 48 hours of exposure to doxorubicin.

FIGS. 11A-11B show that suppression of LAPTM4B induces cathepsin-dependent cleavage of Bid, Caspase-3 and PARD in breast cancer cells that over express LAPTM4B. FIG. 11A shows the results of BT549 cells transfected with a scrambled control siRNA (Ctrl) or LAPTM4B-specific siRNA (L4B siRNA) and incubated in medium alone ( - - - ) or with 1 μM doxorubicin (Doxo). Proteins (100 μg per lane) from cytosol preparations were fractionated on SDS-PAGE, and blots were probed with anti-Cathepsin B, D, and anti-actin (as loading control). FIG. 11B shows the results of BT549 cells were pretreated with cathepsin inhibitors, pepstatin A and EST (Cath inh), or pan-caspase inhibitor z-VAD-fmk (Casp inh) followed by transfection with control or LAPTM4B-specific siRNA then treated with medium alone ( - - - ) or 1 μM doxorubicin (Doxo). Proteins (100 μg per lane) from cell lysates were fractionated on SDS-PAGE, and blots were probed with anti-Bid, anti-caspase 3, anti-PARP, and anti-actin antibodies.

FIG. 12A-12D show the effects of modulating LAPT4B expression on autophagy maturation and flux. FIGS. 12A-12B show the results of merged immunofluorescence analyses for autophagosomes (anti-LC3, red), lysosomes (anti-LAMP2, green), and nuclear staining (DAPI, blue) in BT549 cells transfected with a control scramble siRNA (FIG. 12A) and LAPTM4B-specific siRNA (FIG. 12B). Cells were cultured in nutrient rich medium (Normal, upper panels of FIGS. 12A-12B) or in low-serum medium (Starvation, lower panels of FIGS. 12A-12B). FIG. 12C shows the results of BT549 cells transfected with control siRNA were cultured with medium alone ( - - - ) or with the autophagy inhibitor chloroquine (CQ) or BT549 transfected with LAPTM4B-specific siRNA cultured in medium alone (si-L4B). Cells were cultured in nutrient rich medium (Normal, left side) or low serum medium (Starvation, right side). Proteins (100 μg per lane) from cell lysates were fractionated on SDS-PAGE, and blots were probed with anti-LC3, anti-p62, anti-active caspase 3, or anti-PARP. FIG. 12D shows the results of MDA468 cells transfected with control vector were cultured with medium alone ( - - - ) or with chloroquine (CQ); MDA468 cells transfected LAPTM4B-expression vector were cultured in medium alone (L4B) or with chloroquine (L4B+CQ). Cells were cultured in nutrient rich medium (Normal, left side) or low serum medium (Starvation, right side). Proteins (100 μg per lane) from cell lysates were fractionated on SDS-PAGE, and blots were probed with anti-LC3 and anti-p62.

FIGS. 13A-13D show that overexpression of LAPTM4B has no effect on growth in acidic conditions but improves survival and growth in hypoxic conditions. Cell survival inhibition curves are shown for MDA468 cells (FIGS. 13A and 13C) transfected with control GFP vector (open circles), and LAPTM4B vector (closed circles) and BT549 cells (FIGS. 13B and 13D) transfected with scramble control (open circles) and LATPM4B-specific siRNA (closed circles). FIGS. 13A-13B show cell survival curves for cells cultured in acidified medium with decreasing pH levels. FIGS. 13C-13D show cell survival curves for cells cultured in normal oxygen (circles) and 1% O₂ hypoxia (triangles). Percent viable cells relative to time zero is shown on y-axis.

FIGS. 14A-14D show quantitation for LC3II/I and p62/actin from immunoblots in FIGS. 12C-12D. FIGS. 14A-14B shows the results of BT549 cells transfected with control siRNA cultured in medium alone ( - - - ) or with chloroquine (CQ) or transfected with LAPTM4B-specific siRNA cultured in medium alone (si-L4B). FIGS. 14C-14D shows the results of MDA468 cells transfected with control vector cultured in medium alone ( - - - ) or with chloroquine (CQ) or transfected with LAPTM4B-expression vector cultured in medium alone (L4B) or with chloroquine (L4B+CQ). For all panels, cells were cultured in nutrient rich medium (white bars) or low serum medium (black bars). FIGS. 14A and 14C show quantitation of immunoblot showing ratio of LC3II to LC3I. FIGS. 14B and 14D show quantitation of immunoblots showing ratio of p62 to actin.

FIGS. 15A-15C shows the effects of modulating LAPTM4B expression on tolerance to nutrient stress. Nutrient and glucose-dependent cell survival curves are shown for BT549 cells (FIG. 15A) transfected with scramble control ( [symbol] ) and LATPM4B-specific siRNA ( [symbol] ) and MDA468 cells (FIG. 15B) transfected with control GFP vector ( [symbol] ), and LAPTM4B vector ( [symbol] ). The ratio of viable cells at each time point vs. time zero are indicated on y-axis (mean of triplicates±s.d.). P-values from t-test for difference between control and LAPTM4B-manipulated cells are indicated as follows: * P<0.1, ** P<0.03, *** P=0.001. Cells were cultured in nutrient rich medium (left panels), low serum (middle panels), and low glucose medium (right panels). FIG. 15C shows the results of MDA468 cells transfected with control GFP vector (Ctrl), and LAPTM4B vector (L4B) and BT549 cells transfected with scramble control (Ctrl) and LATPM4B-specific siRNA (si-L4B). Cell lines are indicated along the top. Cells were cultured in nutrient rich, low serum, and low glucose medium as indicated above the blot. Proteins (100 μg per lane) from cell lysates were fractionated on SDS-PAGE, and blots were probed with anti-caspase 3, anti-PARP, and anti-actin antibodies.

FIGS. 16A-16E show that overexpression of LAPTM4B promotes increased autophagy and faster tumor growth in vivo. FIG. 16A shows tumor growth of MDA468 expressing control vector ( [symbol] ) or LAPTM4B vector ( [symbol] ) in immunodeficient mice. FIG. 16B shows the results of immunoblotting for His epitope tagged-LAPTM4B in tumor explant lysates derived from MDA468 cells expressing a control vector (left lane) or LAPTM4B vector (right lanes). Full-length LAPTM4B (black arrow) and smaller proteolytic fragments (white arrows) indicate delivery of LAPTM4B to lysosomes. FIG. 16C shows the results of immunoblotting for LC3 and p62 in tumor explant lysates derived from cells expressing a control vector (left lanes, n=4) or LAPTM4B vector (right lanes, n=5). Quantification of LC3_{II} to LC3_{I} (FIG. 16D), p62 to actin (FIG. 16E), and LC3_{II}/LC3_{I} to p62 (FIG. 16F) ratios are shown. Data represent mean±standard deviation of MDA468 xenograft tumors expressing a control vector (white bars, n=4) or LAPTM4B vector (black bars, n=5).

### BRIEF DESCRIPTION OF THE TABLES

Table 1 shows a list of Predictive Analysis of Microarrays (PAM) probes.

Table 2 shows the associations of clinical and pathologic factors to risk of distant cancer recurrence.

Table 3 shows prediction data of pathological complete response to epirubicin and cisplatin by 8q22 genes.

Table 4 shows a list of cancer sample features.

### DETAILED DESCRIPTION OF THE INVENTION

The present invention is based, in part, on the discovery that the amplification of the human 8q22-23 region in tumors and over-expression of genes residing in that chromosomal region, especially including the two genes, LAPTM4B and YWHAZ, is associated with and predictive of resistance (e.g., de novo resistance) to anthracycline-based chemotherapy. Increased copy number, amount, and/or activity of both LAPTM4B and YWHAZ in combination within hyperproliferative cells provides an unexpectedly enhanced ability to predict increased resistance of the hyperproliferative cells to anthracycline-based chemotherapy. Determining decreased or wild type copy number, amount, and/or activity of both LAPTM4B and YWHAZ in combination within hyperproliferative cells provides an unexpectedly enhanced ability to predict decreased resistance (i.e., enhanced efficacy) of the hyperproliferative cells to anthracycline-based chemotherapy. Without being bound by theory, it is believed that increased LAPTM4B copy number, amount, and/or activity interferes with nuclear accumulation of anthracyclines and thus anthracycline resistance due to reduced ability to induce DNA damage. This anthracycline resistance is synergistically enhanced by the anti-apoptotic functions of YWHAZ.

Using the methods and compositions described herein, statistically significant models of predicted resistance of subjects to anthracycline-based chemotherapy evaluated in such terms as subject relapse, disease free survival, metastasis free survival, overall survival and/or disease progression can be developed and utilized to assist subjects and clinicians in determining suitable treatment options to be included in the therapeutic regimen. Thus, the methods and compositions of the present invention may be used as a predictive marker of outcome of a anthracycline treatment, thereby enabling the physician to determine if said treatment is of benefit to a subject.

Using the methods and compositions as described herein, subject survival may also be evaluated before or during treatment for a cell hyperproliferative disorder suitable for treatment with anthracyclines, in order to provide critical information to the subject and clinician as to the likely progression of the disease when treated by means of a therapy comprising an anthracycline. It will be appreciated, therefore, that the methods and compositions exemplified herein can serve to improve a subject's quality of life and odds of treatment success by allowing both subject and clinician a more accurate assessment of the subject's treatment options. It will also be appreciated that the methods and compositions described herein may be combined with other treatment regimes and/or other predictive biomarkers and methods of using same.

### I. DEFINITIONS

The articles “a” and “an” are used herein to refer to one or to more than one (i.e. to at least one) of the grammatical object of the article. By way of example, “an element” means one element or more than one element.

The term “altered amount” of a marker or “altered level” of a marker refers to increased or decreased copy number of a minimal common region (MCR) or marker, e.g., 8q22-23 or a sub-region therein, such as region 98,778K to 101,970K on human chromosome 8 of the Genome Reference Consortium Human genome build 37 (hereinafter, GRCh37), and/or increased or decreased expression level of a particular marker gene or genes in a cancer sample, as compared to the expression level or copy number of the marker in a control sample. The term “altered amount” of a marker also includes an increased or decreased protein level of a marker in a sample, e.g., a cancer sample, as compared to the protein level of the marker in a normal, control sample.

The “amount” of a marker, e.g., expression or copy number of a marker or MCR, or protein level of a marker, in a subject is “significantly” higher or lower than the normal amount of a marker or MCR, if the amount of the marker is greater or less, respectively, than the normal level by an amount greater than the standard error of the assay employed to assess amount, and preferably at least twice, and more preferably three, four, five, ten or more times that amount. Alternately, the amount of the marker or MCR in the subject can be considered “significantly” higher or lower than the normal amount if the amount is at least about two, and preferably at least about three, four, or five times, higher or lower, respectively, than the normal amount of the marker or MCR.

The term “altered level of expression” of a marker or MCR refers to an expression level or copy number of a marker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker or MCR in a control sample (e.g., sample from a healthy subject not having the associated disease) and preferably, the average expression level or copy number of the marker or MCR in several control samples.

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors.

The term “altered structure” of a marker refers to the presence of mutations or allelic variants within the marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

The term “altered subcellular localization” of a marker refers to the mislocalization of the marker within a cell relative to the normal localization within the cell e.g., within a healthy and/or wild-type cell. An indication of normal localization of the marker can be determined through an analysis of subcellular localization motifs known in the field that are harbored by marker polypeptides.

As used herein, “anthracyclines” generally refer to a large group of compounds having at least a tetrahydronaphthacenedione ring structure attached by a glycosidic linkage to a sugar molecule. Commonly used anthracyclines include but are not limited to mitoxantrone, doxorubicin, aclarubicin, daunorubicin, epirubicin and idarubicin. Anthracyclines can form noncovalent DNA intercalation interactions, covalent DNA adducts, inhibit topoisomerase II (topo II) enzyme activity, and/or have free radical effects on cellular membranes and DNA. Structural diversity of anthracyclines is generated by modifications of the backbone including a large number of different side chains (see, e.g., Henry, 1976, Cancer Chemotherapy, ACS Symposium Series, 15-57; Nagy et al., 1996, Proc. Natl. Acad. Sci. USA, 93: 2464-9; Bakina et al., 1999, Anti-Cancer Drug Design, 14: 507-15; Perrin et al., 1999, Nucleic Acids Research, p. 1781; U.S. Pat. Nos. 4,301,277; 4,314,054; 4,464,529; 4,585,859; 4,591,637; 4,826,964; 5,843,903; and U.S. Pat. Nos. 6,184,374; 5,962,216; 5,196,522; 6,218,519; 6,433,150; PCT publication No. WO 98/13059; and Eur. Pat. No. EP 02/90744).

Unless otherwise specified herein, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., LAPTM4B polypeptide or fragment thereof or YWHAZ polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. Preferably, antibodies of the invention bind specifically or substantially specifically to LAPTM4B and YWHAZ polypeptides or fragments thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “body fluid” refers to fluids that are excreted or secreted from the body as well as fluid that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. In one embodiment, the present invention is used in the analysis of breast cancer subjects. In another embodiment, efficacy of anthracycline treatment for various subtypes of breast cancer (including, but not limited to, estrogen receptor negative (ER−); v-erb-b2 erythroblastic leukemia viral oncogene homolog-2 negative (HER2−); ER−, HER2+; ER+, HER2−; ER+, HER2+; and ER−, progesterone negative (PR−), HER2− (Triple Negative); all subtypes of which can exhibit 8q22 gain) are predicted and treated based upon analysis of MCRs and markers of the present invention.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, the term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The “copy number of a gene” or the “copy number of a marker” refers to the number of DNA sequences in a cell encoding a particular gene product. Generally, for a given gene, a mammal has two copies of each gene. The copy number can be increased, however, by gene amplification or duplication, or reduced by deletion.

The “normal” copy number of a marker or MCR or “normal” level of expression of a marker is the level of expression, copy number of the marker, or copy number of the MCR, in a biological sample, e.g., a sample containing tissue, whole blood, serum, plasma, buccal scrape, saliva, cerebrospinal fluid, urine, stool, and bone marrow, from a subject, e.g., a human, not afflicted with cancer.

As used herein, the term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

As used herein, the term “diagnostic marker” includes markers listed herein which are useful in the diagnosis of cancer, e.g., over- or under-activity, emergence, expression, growth, remission, recurrence or resistance of tumors before, during or after therapy. The predictive functions of the marker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression (e.g., by ISH, Northern Blot, or qPCR), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human cancers types or cancer samples; (2) its presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its presence or absence in clinical subset of subjects with cancer (e.g., those responding to a particular therapy or those developing resistance). Diagnostic markers also include “surrogate markers,” e.g., markers which are indirect markers of cancer progression.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

As used herein, the term “inhibit” includes the decrease, limitation, or blockage, of, for example a particular action, function, or interaction.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

As used herein, the term “interaction”, when referring to an interaction between two molecules, refers to the physical contact (e.g., binding) of the molecules with one another. Generally, such an interaction results in an activity (which produces a biological effect) of one or both of said molecules.

An “isolated antibody”, as used herein, is intended to refer to an antibody that is substantially free of other antibodies having different antigenic specificities (e.g., an isolated antibody that specifically binds LAPTM4B polypeptide or a fragment thereof, or YWHAZ polypeptide or a fragment thereof, is substantially free of antibodies that specifically bind antigens other than said polypeptide or a fragment thereof). Moreover, an isolated antibody may be substantially free of other cellular material and/or chemicals.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of LAPTM4B polypeptide or a fragment thereof, or YWHAZ polypeptide or a fragment thereof, in which the protein is separated from cellular components of the cells from which it is isolated or recombinantly produced. In one embodiment, the language “substantially free of cellular material” includes preparations of target protein (e.g., LAPTM4B polypeptide or a fragment thereof, or YWHAZ polypeptide or a fragment thereof), having less than about 30% (by dry weight) of non-target protein (also referred to herein as a “contaminating protein”), more preferably less than about 20% of non-target protein, still more preferably less than about 10% of non-target protein, and most preferably less than about 5% non-target protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g. a package or container) comprising at least one reagent, e.g. a probe or small molecule, for specifically detecting and/or affecting the expression of a marker of the invention. The kit may be promoted, distributed, or sold as a unit for performing the methods of the present invention. The kit may comprise one or more reagents necessary to express a marker of the invention (e.g., LAPTM4B polypeptide or a fragment thereof, or YWHAZ polypeptide or a fragment thereof). In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

A “marker” is a gene whose altered level of expression in a tissue or cell from its expression level in normal or healthy tissue or cell is associated with a disease state, such as cancer. A “marker nucleic acid” is a nucleic acid (e.g., mRNA, cDNA) encoded by or corresponding to a marker of the invention. Such marker nucleic acids include DNA (e.g., cDNA) comprising the entire or a partial sequence of any of the nucleic acid sequences set forth in the Sequence Listing or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” is a protein encoded by or corresponding to a marker of the invention. A marker protein comprises the entire or a partial sequence of any of the sequences set forth in the Sequence Listing. The terms “protein” and “polypeptide” are used interchangeably.

A “minimal common region (MCR),” as used herein, refers to a contiguous chromosomal region which displays either gain and amplification (increased copy number) or loss and deletion (decreased copy number) in the genome of a cancer. An MCR includes at least one nucleic acid sequence which has increased or decreased copy number and which is associated with a cancer. In some embodiments, the MCR is human chromosomal region 8q22-23, or a sub-region therein, such as region NC_{—}000008.10 or region 98,778K to 101,970K on human chromosome 8 of the Genome Reference Consortium Human genome build 37 (hereinafter, GRCh37).

The “normal” level of expression of a marker is the level of expression of the marker in cells of a subject, e.g., a human subject, not afflicted with a cancer, e.g., lung, ovarian, pancreatic, liver, breast, prostate, and colon carcinomas, as well as melanoma and multiple myeloma. An “over-expression” or “significantly higher level of expression” of a marker refers to an expression level in a test sample that is greater than the standard error of the assay employed to assess expression, and is preferably at least twice, and more preferably three, four, five or ten times the expression level of the marker in a control sample (e.g., sample from a healthy subjects not having the marker associated disease) and preferably, the average expression level of the marker in several control samples. A “significantly lower level of expression” of a marker refers to an expression level in a test sample that is at least twice, and more preferably three, four, five or ten times lower than the expression level of the marker in a control sample (e.g., sample from a healthy subject not having the marker associated disease) and preferably, the average expression level of the marker in several control samples.

An “overexpression” or “significantly higher level of expression or copy number” of a marker or MCR refers to an expression level or copy number in a test sample that is greater than the standard error of the assay employed to assess expression or copy number, and is preferably at least twice, and more preferably three, four, five or ten or more times the expression level or copy number of the marker or MCR in a control sample (e.g., sample from a healthy subject not afflicted with cancer) and preferably, the average expression level or copy number of the marker or MCR in several control samples.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The “response of a hyperproliferative disorder to chemotherapy” relates to any response of the hyperproliferative disorder to chemotherapy, preferably to a change in tumor mass and/or volume after initiation of neoadjuvant or adjuvant chemotherapy. Hyperproliferative disorder response may be assessed in a neoadjuvant or adjuvant situation where the size of a tumor after systemic intervention can be compared to the initial size and dimensions as measured by CT, PET, mammogram, ultrasound or palpation. Response may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy.

An “RNA interfering agent” as used herein, is defined as any agent which interferes with or inhibits expression of a target gene, e.g., a marker of the invention, by RNA interference (RNAi). Such RNA interfering agents include, but are not limited to, nucleic acid molecules including RNA molecules which are homologous to the target gene, e.g., a marker of the invention, or a fragment thereof, short interfering RNA (siRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated be reference herein).

RNA interfering agents, e.g., siRNA molecules, may be administered to a subject having or at risk for having cancer, to inhibit expression of a marker gene of the invention, e.g., a marker gene which is overexpressed in cancer (such as the markers listed in Table 3) and thereby treat, prevent, or inhibit cancer in the subject.

A “constitutive” promoter is a nucleotide sequence which, when operably linked with a polynucleotide which encodes or specifies a gene product, causes the gene product to be produced in a living human cell under most or all physiological conditions of the cell.

As used herein, “stringent hybridization conditions” involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley and Sons, N.Y.) at Unit 2.10.

As used herein, “subject” refers to any healthy animal, mammal or human, or any animal, mammal or human afflicted with a cancer, e.g., lung, ovarian, pancreatic, liver, breast, prostate, and colon carcinomas, as well as melanoma and multiple myeloma. The term “subject” is interchangeable with “subject”.

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

As used herein, the term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

An “underexpression” or “significantly lower level of expression or copy number” of a marker or MCR refers to an expression level or copy number in a test sample that is greater than the standard error of the assay employed to assess expression or copy number, but is preferably at least twice, and more preferably three, four, five or ten or more times less than the expression level or copy number of the marker or MCR in a control sample (e.g., sample from a healthy subject not afflicted with cancer) and preferably, the average expression level or copy number of the marker or MCR in several control samples.

As used herein, the term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG,GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

### II. DESCRIPTION

The methods and compositions of the present disclosure relate to detection of expression and/or activity of a marker or MCR, e.g., 8q22-23 or a sub-region therein, such as region NC_{—}000008.10 or region 98,778K to 101,970K on human chromosome 8 of the Genome Reference Consortium Human genome build 37 (hereinafter, GRCh37). In one embodiment, the marker is the gene referred to herein as the LAPTM4B gene or a fragment thereof, e.g., a biologically active fragment thereof, as well as to the detection of expression and/or activity of gene products encoded by the LAPTM4B gene (i.e., a “LAPTM4B gene product”) or fragments thereof, e.g., biologically active fragments thereof. The methods and compositions of the present disclosure can utilize the LAPTM4B gene or gene sequence or fragments thereof, as well as gene products of the LAPTM4B gene, e.g., antibodies which specifically bind to such LAPTM4B gene products, or fragments thereof. Sequences, splice variants, and structures of LAPTM4B gene and gene products have been described in the art. See, for example, the Gene Cards.com website available on the world wide web at genecards.org/cgi-bin/carddisp.pl?gene=LAPTM4B; Shao et al. (2003) Oncogene 22, 5060-5069; and Kasper et al. (2005) Cancer Lett. 224:93-103. LAPTM4B gene and gene products from many species are known and include, for example, canine LAPTM4B (NCBI Accession XM_{—}532277.2 and XP_{—}532277.2), chimpanzee LAPTM4B (NCBI Accession XM_{—}508462.2 and XP_{—}508462.2), cow LAPTM4B (NCBI Accession NM_{—}205802.1 and NP_{—}991371.1), rat LAPTM4B (NCBI Accession NM_{—}001013174.1 and NP_{—}001013192.1), and mouse LAPTM4B (NCBI Accession NM_{—}033521.3 and NP_{—}277056.1). Human LAPTM4B sequences include those listed below.

| LAPTM4B coding nucleic acid sequence (NCBI Accession NM_018407.4): | |
|---|---|
| 1 | atgacgtcac ggactcgggt cacatggcca agtccgcccc gccccctccc cgtccccgcc |
| 61 | gctgcagcgg tcgccttcgg agcgaagggt accgacccgg cagaagctcg gagctctcgg |
| 121 | ggtatcgagg aggcaggccc gcgggcgcac gggcgagcgg gccgggagcc ggagcggcgg |
| 181 | aggagccggc agcagcggcg cggcgggctc caggcgaggc ggtcgacgct cctgaaaact |
| 241 | tgcgcgcgcg ctcgcgccac tgcgcccgga gcgatgaaga tggtcgcgcc ctggacgcgg |
| 301 | ttctactcca acagctgctg cttgtgctgc catgtccgca ccggcaccat cctgctcggc |
| 361 | gtctggtatc tgatcatcaa tgctgtggta ctgttgattt tattgagtgc cctggctgat |
| 421 | ccggatcagt ataacttttc aagttctgaa ctgggaggtg actttgagtt catggatgat |
| 481 | gccaacatgt gcattgccat tgcgatttct cttctcatga tcctgatatg tgctatggct |
| 541 | acttacggag cgtacaagca acgcgcagcc tggatcatcc cattcttctg ttaccagatc |
| 601 | tttgactttg ccctgaacat gttggttgca atcactgtgc ttatttatcc aaactccatt |
| 661 | caggaataca tacggcaact gcctcctaat tttccctaca gagatgatgt catgtcagtg |
| 721 | aatcctacct gtttggtcct tattattctt ctgtttatta gcattatctt gacttttaag |
| 781 | ggttacttga ttagctgtgt ttggaactgc taccgataca tcaatggtag gaactcctct |
| 841 | gatgtcctgg tttatgttac cagcaatgac actacggtgc tgctaccccc gtatgatgat |
| 901 | gccactgtga atggtgctgc caaggagcca ccgccacctt acgtgtctgc ctaa |
| LAPTM4B protein sequence (NCBI Accession Number NP_060877.3): | |
| 1 | mtsrtrvtwp spprplpvpa aaavafgakg tdpaearssr gieeagprah gragreperr |
| 61 | rsrqqrrggl qarrstllkt cararatapg amkmvapwtr fysnscclcc hvrtgtillg |
| 121 | vwyliinavv llillsalad pdqynfssse lggdfefmdd anmciaiais llmilicama |
| 181 | tygaykqraa wiipffcyqi fdfalnmlva itvliypnsi qeyirqlppn fpyrddvmsv |
| 241 | nptclvliil lfisiiltfk gyliscvwnc yryingrnss dvlvyvtsnd ttvllppydd |
| 301 | atvngaakep pppyvsa |

In another embodiment, the marker is the gene referred to herein as the YWHAZ gene or a fragment thereof, e.g., a biologically active fragment thereof, as well as to the detection of expression and/or activity of gene products encoded by the YWHAZ gene (i.e., a “YWHAZ gene product”) or fragments thereof, e.g., biologically active fragments thereof. The methods and compositions of the present disclosure can utilize the YWHAZ gene or gene sequence or fragments thereof, as well as gene products of the YWHAZ gene, e.g., antibodies which specifically bind to such YWHAZ gene products, or fragments thereof. Sequences, splice variants; and structures of YWHAZ gene and gene products have been described in the art. See, for example, the Gene Cards.com website available on the world wide web at genecards.org/cgi-bin/carddisp.pl?gene=YWHAZ; Maxwell et al. (2009) J. Biol. Chem. 284, 22379-22389, Lu et al. (2009) Cancer Cell 16, 195-207; Neal et al. (2009) Cancer Res. 69, 3425-3432; Frasor et al. (2006) Cancer Res. 66, 7334-7340. YWHAZ gene and gene products from many species are known and include, for example, canine YWHAZ (NCBI Accession XM_{—}533072.2 and XP_{—}533072.2), chimpanzee YWHAZ (NCBI Accession XM_{—}528202.2 and XP_{—}528202.2), cow YWHAZ (NCBI Accession NM_{—}174814.2 and NP_{—}777239.1), rat YWHAZ (NCBI Accession NM_{—}013011.2 and NP_{—}037143.2), and mouse YWHAZ (NCBI Accession NM_{—}011740.2 and NP_{—}035870.1). Human YWHAZ sequences include those listed below.

| YWHAZ coding nucleic acid sequence (NCBI Accession NM_003406.3): | |
|---|---|
| 1 | atggataaaa atgagctggt tcagaaggcc aaactggccg agcaggctga gcgatatgat |
| 61 | gacatggcag cctgcatgaa gtctgtaact gagcaaggag ctgaattatc caatgaggag |
| 121 | aggaatcttc tctcagttgc ttataaaaat gttgtaggag cccgtaggtc atcttggagg |
| 181 | gtcgtctcaa gtattgaaca aaagacggaa ggtgctgaga aaaaacagca gatggctcga |
| 241 | gaatacagag agaaaattga gacggagcta agagatatct gcaatgatgt actgtctctt |
| 301 | ttggaaaagt tcttgatccc caatgcttca caagcagaga gcaaagtctt ctatttgaaa |
| 361 | atgaaaggag attactaccg ttacttggct gaggttgccg ctggtgatga caagaaaggg |
| 421 | attgtcgatc agtcacaaca agcataccaa gaagcttttg aaatcagcaa aaaggaaatg |
| 481 | caaccaacac atcctatcag actgggtctg gcccttaact tctctgtgtt ctattatgag |
| 541 | attctgaact ccccagagaa agcctgctct cttgcaaaga cagcttttga tgaagccatt |
| 601 | gctgaacttg atacattaag tgaagagtca tacaaagaca gcacgctaat aatgcaatta |
| 661 | ctgagagaca acttgacatt gtggacatcg gatacccaag gagacgaagc tgaagcagga |
| 721 | gaaggagggg aaaattaa |
| YWHAZ protein sequence (NCBI Accession Number NP_003397.1): | |
| 1 | mdknelvqka klaeqaeryd dmaacmksvt eqgaelsnee rnllsvaykn vvgarrsswr |
| 61 | vvssieqkte gaekkqqmar eyrekietel rdicndvlsl lekflipnas qaeskvfylk |
| 121 | mkgdyyryla evaagddkkg ivdqsqqayq eafeiskkem qpthpirlgl alnfsvfyye |
| 181 | ilnspekacs laktafdeai aeldtlsees ykdstlimql lrdnitlwts dtqgdeaeag |
| 241 | eggen |

The present invention further provides variants, fragments, and functionally similar homologs of LAPTM4B and YWHAZ for use in the methods describer further herein. For example, a nucleic acid molecule of the present invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acid molecules encoding a protein which corresponds to a marker of the invention, and thus encode the same protein.

In addition to the nucleotide sequences described in the Tables, Figures, and Sequence Listing described herein, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene, including, but not limited to, the genes listed in Table 2A, Table 2B, Table 13A, Table 13B, and FIG. 19, can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989).

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of one of the proteins which correspond to the markers of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid molecule of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl)uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

### III. LAPTM4B AND YWHAZ ANTIBODIES

An isolated LAPTM4B or YWHAZ polypeptide or a fragment thereof (or a nucleic acid encoding such a polypeptide), can be used as an immunogen to generate antibodies that bind to said immunogen, using standard techniques for polyclonal and monoclonal antibody preparation. A full-length LAPTM4B or YWHAZ polypeptide can be used, or alternatively, the disclosure relates to antigenic peptide fragments of LAPTM4B or YWHAZ polypeptide for use as immunogens. An antigenic peptide of LAPTM4B or YWHAZ comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In one embodiment, an antibody binds substantially specifically to a LAPTM4B or YWHAZ polypeptide, or a fragment thereof. In a preferred embodiment, an antibody binds to a LAPTM4B or YWHAZ polypeptide, or a fragment thereof, and blocks the interaction between a LAPTM4B or YWHAZ polypeptide or a fragment thereof and its natural binding partner(s) or a fragment(s) thereof.

A LAPTM4B or YWHAZ immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well known protocols used for fusing lymphocytes and immortalized cell lines can be applied for the purpose of generating an anti-LAPTM4B or anti-YWHAZ monoclonal antibody (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal antibody specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-LAPTM4B or anti-YWHAZ polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743.

Additionally, fully human antibodies could be made against a LAPTM4B or YWHAZ immunogen. Fully human antibodies can be made in mice that are transgenic for human immunoglobulin genes, e.g. according to Hogan, et al., “Manipulating the Mouse Embryo: A Laboratory Manuel,” Cold Spring Harbor Laboratory. Briefly, transgenic mice are immunized with purified LAPTM4B or YWHAZ immunogen. Spleen cells are harvested and fused to myeloma cells to produce hybridomas. Hybridomas are selected based on their ability to produce antibodies which bind to the LAPTM4B or YWHAZ immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a LAPTM4B or YWHAZ polypeptide or a fragment thereof. In one embodiment, the bispecific antibody could specifically bind to both a LAPTM4B or YWHAZ polypeptide or a fragment thereof and its natural binding partner(s) or a fragment(s) thereof.

Yet another aspect of the invention pertains to anti-LAPTM4B or anti-YWHAZ antibodies that are obtainable by a process comprising, immunizing an animal with an immunogenic LAPTM4B or YWHAZ polypeptide or an immunogenic portion thereof unique to LAPTM4B or YWHAZ, respectively; and then isolating from the animal antibodies that specifically bind to the polypeptide or a fragment thereof.

In another aspect of this invention, LAPTM4B or YWHAZ polypeptide fragments or variants can be used. In one embodiment, a variegated library of LAPTM4B or YWHAZ variants is generated by combinatorial mutagenesis at the nucleic acid level and is encoded by a variegated gene library. A variegated library of LAPTM4B or YWHAZ variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of LAPTM4B or YWHAZ such as dominant-negative variants (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes LAPTM4B or YWHAZ. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

In one embodiment, the peptide has an amino acid sequence identical or similar to the LAPTM4B or YWHAZ binding site of its natural binding partner(s) or a fragment(s) thereof. In one embodiment, the peptide competes with a LAPTM4B or YWHAZ polypeptide or a fragment thereof for binding its natural binding partner(s) or a fragment(s) thereof.

Peptides can be produced, typically by direct chemical synthesis, and used e.g., as antagonists of the interactions between a LAPTM4B or YWHAZ polypeptide or a fragment thereof and its natural binding partner(s) or a fragment(s) thereof. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, and biochemical properties.

Peptidomimetics (Fauchere, J. (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to peptides useful for diagnostic, prognostic, and/or clinical trial monitoring applications can be used to produce equivalent diagnostic, prognostic, and/or clinical trial monitoring applications. Generally, peptidomimetics are structurally similar to a paradigm polypeptide (i.e., a polypeptide that has a biological or pharmacological activity), such as a human LAPTM4B or YWHAZ polypeptide or a fragment thereof, but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1.979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds. Derivitization (e.g., labeling) of peptidomimetics should not substantially interfere with the desired diagnostic and/or prognostic utility of the peptidomimetic.

These peptides or peptidomimetic molecules can also be chimeric or fusion proteins. As used herein, a “chimeric protein” or “fusion protein” comprises a protein, peptide, or peptidomimetic molecule or a fragment thereof operatively linked to another protein, peptide, or peptidomimetic molecule or a fragment thereof. A “LAPTM4B molecule” or “YWHAZ molecule” refers to a polypeptide having an amino acid sequence corresponding to LAPTM4B or YWHAZ or a fragment thereof, whereas a “non-LAPTM4B molecule” or a “non-YWHAZ molecule” refers to a polypeptide having an amino acid sequence corresponding to a protein which is not substantially homologous to the respective LAPTM4B or YWHAZ molecule, e.g., a protein which is different from the LAPTM4B or YWHAZ molecule, and which is derived from the same or a different organism. Within a LAPTM4B or YWHAZ fusion protein, the LAPTM4B or YWHAZ portion can correspond to all or a portion of a full length LAPTM4B or YWHAZ molecule. Within the chimeric or fusion protein, the term “operatively linked” is intended to indicate that the independent protein, peptide, or peptidomimetic molecules or fragments thereof are fused in-frame to each other in such a way as to preserve functions exhibited when expressed independently of the fusion.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. Preferably, the first peptide consists of a portion of LAPTM4B or YWHAZ that comprises at least one biologically active portion of a LAPTM4B or YWHAZ molecule. In another preferred embodiment, the first peptide consists of a portion of a biologically active molecule. The second peptide can include an immunoglobulin constant region, for example, a human Cγ1 domain or Cγ4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture, typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). A polypeptide encoding nucleic acid can be cloned into such an expression vector such that the fusion moiety is linked in-frame to the LAPTM4B or YWHAZ encoding sequences.

In another embodiment, the fusion protein contains a heterologous signal sequence at its N-terminus. In certain host cells (e.g., mammalian host cells), expression and/or secretion of a polypeptide can be increased through use of a heterologous signal sequence.

The fusion proteins of the invention can be used as immunogens to produce antibodies in a subject. Such antibodies may be used to purify the respective natural polypeptides from which the fusion proteins were generated, or in screening assays to identify polypeptides which inhibit the interactions between a LAPTM4B or YWHAZ polypeptide or a fragment thereof and its natural binding partner(s) or a fragment(s) thereof.

### IV. USES AND METHODS OF THE INVENTION

The LAPTM4B or YWHAZ molecules, e.g., molecules comprising the LAPTM4B or YWHAZ nucleic acid molecules, polypeptides, polypeptide homologues, antibodies, and fragments thereof, as well as inhibitors thereof such as RNA interference or small molecules thereof, described herein can be used in one or more of the following methods: a) predictive medicine and b) treatment of hyperproliferative disorders.

The isolated nucleic acid molecules of the invention can be used, for example, to detect a marker (e.g., LAPTM4B or YWHAZ mRNA or functionally similar homologs thereof, including a fragment or genetic alteration thereof) or MCR (e.g., human chromosomal region 8q22-23, or a sub-region therein, such as region NC_{—}000008.10 or region 98,778K to 101,970K on human chromosome 8 of the Genome Reference Consortium Human genome build 37, of the present invention and as described further below. Moreover, the anti-LAPTM4B or anti-YWHAZ antibodies or fragments thereof of the invention can be used to detect LAPTM4B or YWHAZ polypeptides or fragments thereof.

A. Predictive Medicine

The methods according to the present invention may be used to provide a prediction of subject survival and/or relapse following treatment by means of a therapy comprising at least one anthracyline. In one embodiment, prediction is defined in terms of subject survival and/or relapse. In this embodiment subjects survival times and/or relapse are predicted according to their gene expression or genetic or epigenetic modifications thereof. In this aspect of the invention, subjects can be tested prior to receiving any adjuvant anthracycline treatment.

In another aspect, the predictive methods described herein can be used to determine whether a subject can be administered an agent (e.g., an agonist, antagonist, peptidomimetic, polypeptide, peptide, nucleic acid, small molecule, or other drug candidate comprising or not comprising an anthracycline) to treat a disease or disorder associated with aberrant or unwanted target MCR or marker expression or activity. For example, such methods can be used to determine whether a subject can be effectively treated with an agent, such as a treatment regimen comprising or not comprising an anthracycline, for a cancer. Thus, the present invention provides methods for determining whether a subject can be effectively treated with an agent for a disorder associated with aberrant or unwanted target MCR or marker expression or activity in which a test sample is obtained and target MCR or marker polypeptide or nucleic acid copy number, expression or activity is detected e.g., wherein the abundance of MCR or marker polypeptide or nucleic acid copy number, expression or activity is predictive of whether a subject can effectively be treated with the agent.

It is herein described that aberrant copy number of human chromosomal region 8q22-23, or a sub-region therein, such as region NC_{—}000008.10 or region 98,778K to 101,970K on human chromosome 8 of the Genome Reference Consortium Human genome build 37 and/or aberrant expression of at least one gene selected from the group consisting of LAPTM4B and YWHAZ and functionally similar homologs thereof, including a fragment or genetic alteration thereof (e.g., in regulatory or promoter regions thereof) is predictive of outcome of treatment of cell hyperproliferative disorder subjects wherein said treatment comprises an anthracycline. Such markers thereby provide a novel means for the characterization of cell hyperproliferative disorders. As described herein, determination of the expression of at least one gene selected from the group consisting LAPTM4B and YWHAZ and functionally similar homologs thereof, including a fragment or genetic alteration thereof (e.g., in regulatory or promoter regions thereof) enables the prediction of treatment response of a subject treated with a therapy comprising an anthracycline. In one embodiment, the aberrant expression of both LAPTM4B and YWHAZ is determined. In another embodiment, subjects are analyzed for aberrant copy number or expression of markers described herein prior to receiving any treatment comprising an anthracycline.

As used herein, the term “aberrant” includes a MCR (e.g., human chromosomal region 8q22-23, or a sub-region therein, such as region NC_{—}000008.10 or region 98,778K to 101,970K on human chromosome 8 of CRCh37) copy number or LAPTM4B and/or YWHAZ expression or activity which deviates from the wild type LAPTM4B and/or YWHAZ expression or activity. Aberrant expression or activity includes increased or decreased expression or activity, as well as expression or activity which does not follow the wild type developmental pattern of expression or the subcellular pattern of expression. For example, aberrant LAPTM4B and/or YWHAZ expression or activity is intended to include the cases in which a mutation in the LAPTM4B and/or YWHAZ gene or regulatory sequence thereof causes the LAPTM4B and/or YWHAZ gene to be under-expressed or over-expressed and situations in which such mutations result in a non-functional LAPTM4B and/or YWHAZ polypeptide or a polypeptide which does not function in a wild-type fashion, e.g., a polypeptide which does not interact with a LAPTM4B and/or YWHAZ binding partner(s) or one which interacts with a non-LAPTM4B and/or non-YWHAZ binding partner(s). As used herein, the term “unwanted” includes an unwanted phenomenon involved in a biological response such as immune cell activation. For example, the term unwanted includes a LAPTM4B and/or YWHAZ expression or activity which is undesirable in a subject.

As used herein, functionally similar homologs thereof, including a fragment or genetic alteration thereof include identifying the presence or absence of a genetic alteration characterized by at least one alteration affecting the integrity of a gene encoding a LAPTM4B and/or YWHAZ polypeptide or functionally similar homolog thereof, or the mis-expression of the LAPTM4B and/or YWHAZ gene or functionally similar homologs thereof. For example, such genetic alterations can be detected by ascertaining the existence of at least one of 1) a deletion of one or more nucleotides from a LAPTM4B and/or YWHAZ gene, 2) an addition of one or more nucleotides to a LAPTM4B and/or YWHAZ gene, 3) a substitution of one or more nucleotides of a LAPTM4B and/or YWHAZ gene, 4) a chromosomal rearrangement of a LAPTM4B and/or YWHAZ gene, 5) an alteration in the level of a messenger RNA transcript of a LAPTM4B and/or YWHAZ gene, 6) aberrant modification of a LAPTM4B and/or YWHAZ gene, such as of the methylation pattern of the genomic DNA, 7) the presence of a non-wild type splicing pattern of a messenger RNA transcript of a LAPTM4B and/or YWHAZ gene, 8) a non-wild type level of a LAPTM4B and/or YWHAZ polypeptide, 9) allelic loss or gain of a LAPTM4B and/or YWHAZ gene, and 10) inappropriate post-translational modification of a LAPTM4B and/or YWHAZ polypeptide. As described herein, there are a large number of assays known in the art which can be used for detecting alterations in a LAPTM4B and/or YWHAZ gene.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a LAPTM4B and/or YWHAZ gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a LAPTM4B and/or YWHAZ gene under conditions such that hybridization and amplification of the LAPTM4B and/or YWHAZ gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a LAPTM4B and/or YWHAZ gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in LAPTM4B and/or YWHAZ can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in LAPTM4B and/or YWHAZ can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such genetic mutations in LAPTM4B and/or YWHAZ can be identified in a variety of contexts, including, for example, germline and somatic mutations.

In yet another embodiment, any of a variety of sequencing reactions known in the art can be used to directly sequence the LAPTM4B and/or YWHAZ gene and detect mutations by comparing the sequence of the sample LAPTM4B and/or YWHAZ with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad. Sci. USA 74:5463. It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159). Various next-generation sequencing techniques are known in the art (e.g., in Metzker (2010) Nat. Rev. Genet. 11:31-46; Voelkerding et al. (2009) Clin. Chem. 55:641-658, Dhiman et al. (2009) Expert Rev. Vaccines 8:963-967; and Turner et al. (2009) Mamm. Genome 20:327-338). Nanopore sequencing is reviewed, e.g., in Branton et al. (2008) Nat. Biotech. 26:1146-1153. Furthermore, equipment and reagents for sequencing are commercially available from a large number of suppliers. Exemplary commercial suppliers of next-generation sequencing systems and reagents include Roche/454 (454 sequencing system, 454.corn), Illumina (e.g., Solexa Genome Analyzer, illumina.corn), Applied Biosystems (SOLiD™ system, appliedbiosystems.corn), Helicos BioSciences (HeliScope, helicosbio.com), and Pacific Biosciences (PacBio® and SMRT sequencing, pacificbiosciences.corn). Such high throughput and/or next-generation sequencing reagents, techniques, and methods are well known in the art.

Other methods for detecting mutations in the LAPTM4B and/or YWHAZ gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type LAPTM4B and/or YWHAZ sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in LAPTM4B and/or YWHAZ cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a LAPTM4B and/or YWHAZ sequence, e.g., a wild-type LAPTM4B and/or YWHAZ sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in LAPTM4B and/or YWHAZ genes. For example, single strand conformation polymorphism (SSCP) may be used to detect differences in electrophoretic mobility between mutant and wild type nucleic acids (Orita et al. (1989) Proc Natl. Acad. Sci. USA 86:2766; see also Cotton (1993) Mutat. Res. 285:125-144 and Hayashi (1992) Genet. Anal. Tech. Appl. 9:73-79). Single-stranded DNA fragments of sample and control LAPTM4B and/or YWHAZ nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

In a further aspect, the invention relates to new methods and sequences, which may be used as tools for the selection of suitable treatments of subjects diagnosed with cell hyperproliferative disease based on a prediction of likelihood of relapse, survival or outcome.

The methods described herein may be enabled by means of any analysis of DNA, the expression of a RNA transcribed therefrom, or polypeptide or protein translated from said RNA, preferably by means of mRNA expression analysis or polypeptide expression analysis. Accordingly the present invention also provides prognostic assays and methods, both quantitative and qualitative for detecting the expression of at least one MCR or marker described herein in a subject with a cell hyperproliferative disorder and determining therefrom upon the prediction of outcome of treatment comprising an anthracycline of said subject.

In one embodiment, a change in genomic MCR or marker (e.g., region 98,778K to 101,970K on human chromosome 8 of CRCh37, LAPTM4B, or YWHAZ) copy number is detected. In one embodiment, a biological sample (described further below) is tested for the presence of copy number changes in genomic loci containing the genomic MCR or marker (e.g., region 98,778K to 101,970K on human chromosome 8 of CRCh37, LAPTM4B, or YWHAZ). A copy number of at least 3, 4, 5, 6, 7, 8, 9, or 10 is predictive of poorer outcome of treatment comprising an anthracycline.

Methods of evaluating the copy number of a particular MCR or marker (e.g., region 98,778K to 101,970K on human chromosome 8 of CRCh37, LAPTM4B, or YWHAZ) include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the copy number of a MCR or marker in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl. Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

In another embodiment, detecting or determining expression levels of LAPTM4B and YWHAZ and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject. When obtaining the cells, it is preferable to obtain a sample containing predominantly cells of the desired type, e.g., a sample of cells in which at least about 50%, preferably at least about 60%, even more preferably at least about 70%, 80% and even more preferably, at least about 90% of the cells are of the desired type. Tissue samples can be obtained according to methods known in the art.

It is also possible to obtain a cell sample from a subject, and then to enrich it in the desired cell type. For example, cells can be isolated from other cells using a variety of techniques, such as isolation with an antibody binding to an epitope on the cell surface of the desired cell type. Where the desired cells are in a solid tissue, particular cells can be dissected out, e.g., by microdissection.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in target MCR or marker sequences. Enrichment can be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCRbased techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising of LAPTM4B and/or YWHAZ DNA. Positive hybridization signal is obtained with the sample containing LAPTM4B and/or YWHAZ transcripts. Methods of preparing DNA arrays and their use are well known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to target MCR or marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize target MCR or marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the target MCR or marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting target MCR or marker polypeptide, mRNA, genomic DNA, or fragments thereof, such that the presence of the target MCR or marker polypeptide, mRNA, genomic DNA, or fragments thereof, is detected in the biological sample, and comparing the presence of the target MCR or marker polypeptide, mRNA, genomic DNA, or fragments thereof, in the control sample with the presence of the target MCR or marker polypeptide, mRNA, genomic DNA, or fragments thereof in the test sample.

In another aspect, the present invention further provides for methods for the detection of the presence of the polypeptide encoded by a marker of the present invention in a biological sample obtained from a subject. Aberrant levels of polypeptide expression of the polypeptides encoded by LAPTM4B and YWHAZ and functionally similar homologs thereof, including a fragment or genetic alteration thereof (e.g., in regulatory or promoter regions thereof) are associated with outcome of cell hyperproliferative disorder treatments comprising an anthracycline. Any method known in the art for detecting polypeptides can be used. Such methods include, but are not limited to immunodiffusion, immunoelectrophoresis, immunochemical methods, binder-ligand assays, immunohistochemical techniques, agglutination and complement assays (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired LAPTM4B and/or YWHAZ standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, LAPTM4B and/or YWHAZ in the sample is allowed to react with the corresponding immobilized antibody, radioisotope- or enzyme-labeled anti-LAPTM4B and/or YWHAZ antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring LAPTM4B and/or YWHAZ levels comprises the steps of: contacting a biological specimen with an antibody or variant (e.g., fragment) thereof which selectively binds LAPTM4B and/or YWHAZ, and detecting whether said antibody or variant thereof is bound to said sample and thereby measuring the levels of LAPTM4B and/or YWHAZ.

Enzymatic and radiolabeling of LAPTM4B and/or YWHAZ and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect LAPTM4B and/or YWHAZ according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Anti-LAPTM4B and/or YWHAZ antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of LAPTM4B and/or YWHAZ, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-LAPTM4B and/or YWHAZ antibodies, such as intrabodies, may also be used for imaging purposes, for example, to detect the presence of LAPTM4B and/or YWHAZ in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m. The labeled antibody or antibody fragment will then preferentially accumulate at the location of cells which contain LAPTM4B and/or YWHAZ. The labeled antibody or antibody fragment can then be detected using known techniques.

Antibodies that may be used to detect LAPTM4B and/or YWHAZ include any antibody, whether natural or synthetic, full length or a fragment thereof, monoclonal or polyclonal, that binds sufficiently strongly and specifically to the LAPTM4B and/or YWHAZ polypeptide to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to LAPTM4B and/or YWHAZ relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., LAPTM4B and/or YWHAZ binding fragments, of antibodies. For example, antibody fragments capable of binding to LAPTM4B and/or YWHAZ or portions thereof, including, but not limited to, Fv, Fab, Fab′ and F (ab′) 2 fragments can be used. Such fragments can be produced by enzymatic cleavage or by recombinant techniques. For example, papain or pepsin cleavage can generate Fab or F (ab′) 2 fragments, respectively. Other proteases with the requisite substrate specificity can also be used to generate Fab or F (ab′) 2 fragments. Antibodies can also be produced in a variety of truncated forms using antibody genes in which one or more stop codons have been introduced upstream of the natural stop site. For example, a chimeric gene encoding a F (ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to LAPTM4B and/or YWHAZ other than antibodies are used, such as peptides. Peptides that specifically bind to LAPTM4B and/or YWHAZ can be identified by any means known in the art. For example, specific peptide binders of LAPTM4B and/or YWHAZ can be screened for using peptide phage display libraries.

In addition, the LAPTM4B and/or YWHAZ protein may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18, 151-160; Rowley et al. (2000) Methods 20, 383-397; Kuster and Mann (1998) Curr. Opin. Structural Biol. 8, 393-400). Further, mass spectrometric techniques have been developed that permit at least partial de novo sequencing of isolated proteins (see, e.g., Chait et al. (1993) Science 262, 89-92; Keough et al. (1999) Proc. Natl. Acad. Sci. USA. 96, 7131-7136; reviewed in Bergman (2000) EXS 88, 133-44).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes (see, e.g., Hellenkamp et al., U.S. Pat. No. 5,118,937 and Beavis and Chait, U.S. Pat. No. 5,045,694).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied (see, e.g., Hutchens and Yip, U.S. Pat. No. 5,719,060 and Hutchens and Yip, WO 98/59361). The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually or by computer analysis) to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. ¹³C) thereby permitting the test sample to be mixed with the known control sample in the same mass spectrometry run.

In one embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantification of the amount of the biomolecule present in the first sample. In certain embodiments, the identity of the biomolecules in the first and second sample can also be determined.

B. LAPTM4B and/or YWHAZ-Based Therapeutics for Treating Cancers

Based on the discovery that high LAPTM4B and/or YWHAZ levels in primary tumors is associated with poorer outcomes of adjuvant treatment regimens comprising an anthracycline, it is possible to increase responsiveness to an adjuvant treatment regimen comprising an anthracycline, halt the progression of cancer, and prevent cancer recurrence by inhibiting or reducing the expression level of LAPTM4B and/or YWHAZ or functionally similar homologs thereof in the tumor or tissue of the subject. In one embodiment, a method for treating cancer, such as lung, ovarian, pancreatic, liver, breast, prostate, and colon carcinomas, comprises reducing the level of expression of LAPTM4B and/or YWHAZ. A method may include reducing the expression of a LAPTM4B and/or YWHAZ gene, reducing the amount of LAPTM4B and/or YWHAZ protein, or inhibiting the activity of a LAPTM4B and/or YWHAZ protein. In a method for treatment, one may reduce LAPTM4B and/or YWHAZ levels or activity in a tumor, e.g., a primary tumor. In a method for preventing cancer recurrence, one may reduce LAPTM4B and/or YWHAZ levels or activity in tissue likely to develop cancer, e.g., tissue that exhibits high levels of LAPTM4B and/or YWHAZ expression.

Prophylaxis may be appropriate even at very early stages of the disease, to prevent recurrent tumorigenesis or metastasis. Thus, administration of an agent that reduces LAPTM4B and/or YWHAZ levels or activity may be effected as soon as cancer is diagnosed, and treatment continued for as long as is necessary, generally until the threat of the disease has been removed. Such treatment may also be used prophylactically in individuals at high risk for development of certain cancers, e.g., breast cancer.

1. RNAi Technology

In one embodiment, LAPTM4B and/or YWHAZ levels are decreased by administration of or expression in a subject, e.g., in cells or a tissue of the subject, of one or more siRNAs.

Isolated RNA molecules specific to LAPTM4B and/or YWHAZ mRNA, which mediate RNAi, are antagonists useful in the method of the present invention (see, e.g., U.S. Patent Application Nos: 20030153519A1; 20030167490A1; and U.S. Pat. Nos. 6,506,559; 6,573,099, which are herein incorporated by reference in their entirety).

In one embodiment, the RNA is comprised of, or capable of being cleaved to, short interfering or small interfering RNAs (siRNAs). The term “short interfering RNAs (siRNA)” as used herein is intended to refer to any nucleic acid molecule capable of mediating RNAi or gene silencing. The term siRNA is intended to encompass various naturally generated or synthetic compounds, with RNAi function. Such compounds include, without limitation, duplex synthetic oligonucleotides, of about 21 to 23 base pairs with terminal overlaps of 2 or 3 base pairs; hairpin structures of one oligonucleotide chain with sense and complementary, hybridizing, segments of 21-23 base pairs joined by a loop of 3-5 base pairs; and various genetic constructs leading to the expression of the preceding structures or functional equivalents. Such genetic constructs are usually prepared in vitro and introduced in the test system, but can also include siRNA from naturally occurring siRNA precursors coded by the genome of the host cell or animal.

It is not a requirement that the siRNA be comprised solely of RNA. In one embodiment, the siRNA comprises one or more chemical modifications and/or nucleotide analogues. The modification and/or analogue may be any modification and/or analogue, respectively, that does not negatively affect the ability of the siRNA to inhibit LAPTM4B and/or YWHAZ expression. The inclusion of one or more chemical modifications and/or nucleotide analogues in an siRNA may be used to prevent or slow nuclease digestion, and in turn, create a more stable siRNA for practical use. Chemical modifications and/or nucleotide analogues which stabilize RNA are known in the art. Phosphorothioate derivatives, which include the replacement of non-bridging phosphoryl oxygen atoms with sulfur atoms, are one example of analogues showing increased resistance to nuclease digestion. Sites of the siRNA which may be targeted for chemical modification include the loop region of a hairpin structure, the 5′ and 3′ ends of a hairpin structure (e.g. cap structures), the 3′ overhang regions of a double-stranded linear siRNA, the 5′ or 3′ ends of the sense strand and/or antisense strand of a linear siRNA, and one or more nucleotides of the sense and/or antisense strand.

As used herein, the term siRNA is intended to be equivalent to any term in the art defined as a molecule capable of mediating sequence-specific RNAi. Such equivalents include, for example, double-stranded RNA (dsRNA), microRNA (mRNA), short hairpin RNA (shRNA), short interfering oligonucleotide, and post-transcriptional gene silencing RNA (ptgsRNA).

siRNAs may be introduced into cells to suppress gene expression for therapeutic or prophylactic purposes as described in International Publication Number WO 0175164. Such molecules may be introduced into cells to suppress gene expression for therapeutic or prophylactic purposes as described in various patents, patent applications and papers. Publications herein incorporated by reference, describing RNAi technology include, but are not limited to, the following: U.S. Pat. No. 6,686,463, U.S. Pat. No. 6,673,611, U.S. Pat. No. 6,623,962, U.S. Pat. No. 6,506,559, U.S. Pat. No. 6,573,099, and U.S. Pat. No. 6,531,644; International Publication Numbers WO04061081; WO04052093; WO04048596; WO04048594; WO04048581; WO04048566; WO04046320; WO04044537; WO04043406; WO04033620; WO04030660; WO04028471; WO 0175164. Papers which describe the methods and concepts for the optimal use of these compounds include, but are not limited to, the following: Brummelkamp Science 296: 550-553 (2002); Caplen Expert Opin. Biol. Ther. 3:575-86 (2003); Brummelkamp, Science Express 21 Mar. 3 1-6 (2003); Yu Proc Natl Acad Sci USA 99:6047-52 (2002); Paul, Nature Biotechnology 29:505-8 (2002); Paddison, Proc Natl Acad Sci USA 99:1443-8 (2002); Brummelkamp, Nature 424: 797-801 (2003); Brummelkamp, Science 296: -550-3 (2003); Sui, Proc Natl Acad Sci USA 99: 5515-20 (2002); Paddison, Genes and Development 16:948-58 (2002).

A composition comprising an siRNA effective to inhibit LAPTM4B and/or YWHAZ expression may include an RNA duplex comprising a sense sequence of LAPTM4B and/or YWHAZ. In this embodiment, the RNA duplex comprises a first strand comprising a sense sequence of LAPTM4B and/or YWHAZ and a second strand comprising a reverse complement of the sense sequence of the LAPTM4B and/or YWHAZ. In one embodiment the sense sequence of LAPTM4B and/or YWHAZ comprises of from 10 to 25 nucleotides in length. In another embodiment, the sense sequence of LAPTM4B and/or YWHAZ comprises of from 19 to 25 nucleotides in length. In yet another embodiment, the sense sequence of LAPTM4B and/or YWHAZ comprises of from 21 to 23 nucleotides in length. The sense sequence of LAPTM4B and/or YWHAZ can comprises a sequence of LAPTM4B and/or YWHAZ containing a translational start site or a portion of LAPTM4B and/or YWHAZ sequence within the first 400 nucleotides of the human LAPTM4B and/or YWHAZ mRNA.

In another embodiment, a composition comprising an siRNA effective to inhibit LAPTM4B and/or YWHAZ expression may comprise in a single molecule a sense sequence of LAPTM4B and/or YWHAZ, the reverse complement of the sense sequence of LAPTM4B and/or YWHAZ, and an intervening sequence enabling duplex formation between the sense and reverse complement sequences. The sense sequence of LAPTM4B and/or YWHAZ may comprise 10 to 25 nucleotides in length, 19 to 25 nucleotides in length, or 21 to 23 nucleotides in length.

It will be readily apparent to one of skill in the art that an siRNA of the present invention may comprise a sense sequence of LAPTM4B and/or YWHAZ or the reverse complement of the sense sequence of LAPTM4B and/or YWHAZ which is less than perfectly complementary to each other or to the targeted region of LAPTM4B and/or YWHAZ. In other words, the siRNA may comprise mismatches or bulges within the sense or reverse complement sequence. In one aspect, the sense sequence or its reverse complement may not be entirely contiguous. The sequence or sequences may comprise one or more substitutions, deletions, and/or insertions. The only requirement of the present invention is that the siRNA sense sequence possess enough complementarity to its reverse complement and to the targeted region of LAPTM4B and/or YWHAZ to allow for RNAi activity. It is an object of the present invention, therefore, to provide for sequence modifications of an siRNA of the present invention that retain sufficient complementarity to allow for RNAi activity. One of skill in the art may predict that a modified siRNA composition of the present invention will work based on the calculated binding free energy of the modified sequence for the complement sequence and targeted region of LAPTM4B and/or YWHAZ. Methods for calculating binding free energies for nucleic acids and the effect of such values on strand hybridization are known in the art.

A wide variety of delivery systems are available for use in delivering an siRNA of the present invention to a target cell in vitro and in vivo. An siRNA of the present invention may be introduced directly or indirectly into a cell in which LAPTM4B and/or YWHAZ inhibition is desired. An siRNA may be directly introduced into a cell by, for example, injection. As such, it is an object of the invention to provide for a composition comprising an siRNA effective to inhibit LAPTM4B and/or YWHAZ in injectable, dosage unit form. An siRNA of the present invention may be injected intravenously or subcutaneously, as an example, for therapeutic use in conjunction with the methods and compositions of the present invention. Such treatment may include intermittent or continuous administration until therapeutically effective levels are achieved to inhibit LAPTM4B and/or YWHAZ expression in the desired tissue.

Indirectly, an expressible DNA sequence or sequences encoding the siRNA may be introduced into a cell and the siRNA, thereafter, transcribed from the DNA sequence or sequences. It is an object of the present invention, therefore, to provide for compositions comprising a DNA sequence or sequences which encode an siRNA effective to inhibit LAPTM4B and/or YWHAZ expression.

A DNA composition of the present invention comprises a first DNA sequence which encodes a first RNA sequence comprising a sense sequence of LAPTM4B and/or YWHAZ and a second DNA sequence which encodes a second RNA sequence comprising the reverse complement of the sense sequence of LAPTM4B and/or YWHAZ. The first and second RNA sequences, when hybridized, form an siRNA duplex capable of forming an RNA-induced silencing complex, the RNA-induced silencing complex being capable of inhibiting LAPTM4B and/or YWHAZ expression. The first and second DNA sequences may be chemically synthesized or synthesized by PCR using appropriate primers to LAPTM4B and/or YWHAZ. Alternatively, the DNA sequences may be obtained by recombinant manipulation using cloning technology, which is well known in the art. Once obtained, the DNA sequences may be purified, combined, and then introduced into a cell in which LAPTM4B and/or YWHAZ inhibition is desired. Alternatively, the sequences may be contained in a single vector or separate vectors and the vector or vectors introduced into the cell in which LAPTM4B and/or YWHAZ inhibition is desired.

Delivery systems available for use in delivering a DNA composition of the present invention to a target cell include, for example, viral and non-viral systems. Examples of suitable viral systems include, for example, adenoviral vectors, adeno-associated virus, lentivirus, poxvirus, retroviral vectors, vaccinia, herpes simplex virus, HIV, the minute virus of mice, hepatitis B virus and influenza virus. Non-viral delivery systems may also be used, for example using, uncomplexed DNA, DNA-liposome complexes, DNA-protein complexes and DNA-coated gold particles, bacterial vectors such as salmonella, and other technologies such as those involving VP22 transport protein, Co-X-gene, and replicon vectors. A viral or non-viral vector in the context of the present invention may express the antigen of interest.

2. Antisense Technology

In another embodiment, the level of LAPTM4B and/or YWHAZ is reduced or decreased by administration or the expression of antisense molecules in a subject or tissue or cell thereof.

Gene expression can be controlled through triple-helix formation or antisense DNA or RNA, both of which methods are based on binding of a polynucleotide to DNA or RNA. An antisense nucleic acid molecule which is complementary to a nucleic acid molecule encoding LAPTM4B and/or YWHAZ can be designed based upon the isolated nucleic acid molecules encoding LAPTM4B and/or YWHAZ. An antisense nucleic acid molecule can comprise a nucleotide sequence which is complementary to a coding strand of a nucleic acid, e.g. complementary to an mRNA sequence, constructed according to the rules of Watson and Crick base pairing, and can hydrogen bond to the coding strand of the nucleic acid. The antisense sequence complementary to a sequence of an mRNA can be complementary to a sequence in the coding region of the mRNA or can be complementary to a 5′ or 3′ untranslated region of the mRNA. Furthermore, an antisense nucleic acid can be complementary in sequence to a regulatory region of the gene encoding the mRNA, for instance, a transcription initiation sequence or regulatory element. In one embodiment, an antisense nucleic acid complementary to a region preceding or spanning the initiation codon or in the 3′ untranslated region of an mRNA is used. An antisense nucleic acid can be designed based upon the nucleotide sequence of LAPTM4B and/or YWHAZ. A nucleic acid is designed which has a sequence complementary to a sequence of the coding or untranslated region of the shown nucleic acid. Alternatively, an antisense nucleic acid can be designed based upon sequences of the LAPTM4B and/or YWHAZ gene, which can be identified by screening a genomic DNA library with an isolated nucleic acid of the invention. For example, the sequence of an important regulatory element can be determined by standard techniques and a sequence which is antisense to the regulatory element can be designed.

The antisense nucleic acids and oligonucleotides of the invention can be constructed using chemical synthesis and enzymatic ligation reactions using procedures known in the art. The antisense nucleic acid or oligonucleotide can be chemically synthesized using naturally occurring nucleotides or variously modified nucleotides designed to increase the biological stability of the molecules or to increase the physical stability of the duplex formed between the antisense and sense nucleic acids. For example, phosphorothioate derivatives and acridine substituted nucleotides can be used. Alternatively, the antisense nucleic acids and oligonucleotides can be produced biologically using an expression vector into which a nucleic acid has been subcloned in an antisense orientation (i.e. nucleic acid transcribed from the inserted nucleic acid will be of an antisense orientation to a target nucleic acid of interest). The antisense expression vector is introduced into cells in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1 (1) 1986.

In addition, ribozymes can be used to inhibit in vitro expression of LAPTM4B and/or YWHAZ. For example, the nucleic acids of the invention can further be used to design ribozymes which are capable of cleaving a single-stranded nucleic acid encoding a LAPTM4B and/or YWHAZ protein, such as a LAPTM4B and/or YWHAZ mRNA transcript. A catalytic RNA (ribozyme) having ribonuclease activity can be designed which has specificity for an mRNA encoding LAPTM4B and/or YWHAZ based upon the sequence of a nucleic acid of the invention. For example, a derivative of a Tetrahymena L-19 IVS RNA can be constructed in which the base sequence of the active site is complementary to the base sequence to be cleaved in a LAPTM4B and/or YWHAZ-encoding mRNA (see, e.g., Cech et al., U.S. Pat. No. 4,987,071; Cech, et al., U.S. Pat. No. 5,116,742). Alternatively, a nucleic acid of the invention could be used to select a catalytic RNA having a specific ribonuclease activity from a pool of RNA molecules (see, e.g., Bartel and Szostak (1993) Science 261, 1411-1418). RNA-mediated interference (RNAi) (Fire et al. (1998) Nature 391, 806-811) may also be used.

3. LAPTM4B and/or YWHAZ Blocking Antibodies or Aptamers

In yet another embodiment, LAPTM4B and/or YWHAZ levels are reduced by administration to or expression in a subject or a cell or tissue thereof, of LAPTM4B and/or YWHAZ blocking antibodies or aptamers.

Antibodies, or their equivalents and derivatives, e.g., intrabodies, or other LAPTM4B and/or YWHAZ antagonists, may be used in accordance with the present invention for the treatment or prophylaxis of cancers. Administration of a suitable dose of the antibody or the antagonist may serve to block the activity of the protein and this may provide a crucial time window in which to treat malignant growth.

A method of treatment involves attachment of a suitable toxin to the antibodies which then target the area of the tumor. Such toxins are well known in the art, and may comprise toxic radioisotopes, heavy metals, enzymes and complement activators, as well as such natural toxins as ricin which are capable of acting at the level of only one or two molecules per cell. It may also be possible to use such a technique to deliver localized doses of suitable physiologically active compounds, which may be used, for example, to treat cancers.

In addition to using antibodies to inhibit LAPTM4B and/or YWHAZ, it may also be possible to use other forms of inhibitors. For example, it may be possible to identify antagonists that functionally inhibit LAPTM4B and/or YWHAZ. In addition, it may also be possible to interfere with the binding of LAPTM4B and/or YWHAZ to target proteins. Other suitable inhibitors will be apparent to the skilled person.

The antibody (or other inhibitors or intrabody) can be administered by a number of methods. One method is set forth by Marasco and Haseltine in PCT WO94/02610, which is incorporated herein by reference. This method discloses the intracellular delivery of a gene encoding the antibody. In one embodiment, a gene encoding a single chain antibody is used. In another embodiment, the antibody would contain a nuclear localization sequence (e.g. an SV40 nuclear localization signal). By this method, one can intracellularly express an antibody, which can block LAPTM4B and/or YWHAZ functioning in desired cells.

Where the present invention provides for the administration of, for example, antibodies to a subject, then this may be by any suitable route. If the tumor is still thought to be, or diagnosed as, localized, then an appropriate method of administration may be by injection direct to the site. Administration may also be by injection, including subcutaneous, intramuscular, intravenous and intradermal injections.

Aptamers can be produced using the methodology disclosed in a U.S. Pat. No. 5,270,163 and WO 91/19813.

4. Other LAPTM4B and/or YWHAZ Inhibitors

Compounds that inhibit the activity of LAPTM4B and/or YWHAZ may also be used. Such compounds include small molecules, e.g., molecules that interact with the active site or a binding site of the protein, e.g., an RNA binding site. Such compounds are well known in the art and additional compounds may be identified according to methods known in the art.

C. Pharmaceutical Formulations

Formulations may be any that are appropriate to the route of administration, and will be apparent to those skilled in the art. The formulations may contain a suitable carrier, such as saline, and may also comprise bulking agents, other medicinal preparations, adjuvants and any other suitable pharmaceutical ingredients. Catheters constitute another mode of administration.

The term “pharmaceutically acceptable” refers to compounds and compositions which may be administered to mammals without undue toxicity. Exemplary pharmaceutically acceptable salts include mineral acid salts such as hydrochlorides, hydrobromides, phosphates, sulfates, and the like; and the salts of organic acids such as acetates, propionates, malonates, benzoates, and the like.

The antibodies, nucleic acids or antagonists of the invention may be administered orally, topically, or by parenteral means, including subcutaneous and intramuscular injection, implantation of sustained release depots, intravenous injection, intranasal administration, and the like. Accordingly, antibodies or nucleic acids of the invention may be administered as a pharmaceutical composition comprising the antibody or nucleic acid of the invention in combination with a pharmaceutically acceptable carrier. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol Registered TM, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, Silvadene Registered TM (Marion), Aquaphor Registered TM (Duke Laboratories), and the like. Other topical formulations include aerosols, bandages, and other wound dressings. Alternatively, one may incorporate or encapsulate the compounds in a suitable polymer matrix or membrane, thus providing a sustained-release delivery device suitable for implantation near the site to be treated locally. Other devices include indwelling catheters and devices such as the Alzet Registered TM minipump. Ophthalmic preparations may be formulated using commercially available vehicles such as Sorbi-care Registered TM (Allergan), Neodecadron Registered TM (Merck, Sharp & Dohme), Lacrilube Registered TM, and the like, or may employ topical preparations such as that described in U.S. Pat. No. 5,124,155, incorporated herein by reference. Further, one may provide an antagonist in solid form, especially as a lyophilized powder. Lyophilized formulations typically contain stabilizing and bulking agents, for example human serum albumin, sucrose, mannitol, and the like. A thorough discussion of pharmaceutically acceptable excipients is available in Remington's Pharmaceutical Sciences (Mack Pub. Co.).

The amount of antibody, nucleic acid or inhibitor required to treat any particular disorder will of course vary depending upon the nature and severity of the disorder, the age and condition of the subject, and other factors readily determined by one of ordinary skill in the art.

### V. KITS

The invention also encompasses kits for detecting the presence of a LAPTM4B and/or YWHAZ nucleic acid, polypeptide, or fragments thereof, in a biological sample. For example, the kit can comprise a labeled compound or agent capable of detecting a target MCR or marker nucleic acid, polypeptide, or fragments thereof in a biological sample; means for determining the amount of the target MCR or marker nucleic acid, polypeptide, or fragments thereof in the sample; and means for comparing the amount of the target MCR or marker nucleic acid, polypeptide, or fragments thereof in the sample with a standard. The compound or agent can be packaged in a suitable container. The kit can further comprise instructions for using the kit to detect the target MCR or marker nucleic acid, polypeptide, or fragments thereof. In a preferred embodiment, the kit for use in determining treatment strategy for a subject with a cell hyperproliferative disorder comprises (a) a plurality of oligonucleotides or polynucleotides able to hybridize under stringent or moderately stringent conditions to the transcription products of at least one gene selected from a target MCR or marker; (b) a container suitable for containing the oligonucleotides or polynucleotides and a biological sample of the subject comprising the transcription products wherein the oligonucleotides or polynucleotide can hybridise under stringent or moderately stringent conditions to the transcription products, (c) means to detect the hybridisation of (b); and optionally, (d) instructions for use and interpretation of the kit results. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR. In another preferred embodiment, a kit according to the embodiments of the present invention is used for the determination of expression step.

### EXAMPLES

### Example 1

### Materials and Methods Used in Examples 2-6

A. Cohort

Primary breast tumors of 115 subjects were obtained from the U.S. National Cancer Institute-Harvard Breast Specialized Program Of Research Excellence blood and tissue repository under protocols approved by the DF/HCC Institutional Review Board, with informed consent from subjects. Affymetrix U133 plus 2 gene expression array analysis was performed as previously described (Matros et al. (2005) Breast Cancer Res. Treat. 91, 179-186; Lu et al. (2008) Breast Cancer Res. Treat. 108, 191-201). A subset of 85 tumors was represented in tissue microarrays and these were used for FISH analysis according to methods described further below. 50 of the cases were analyzed by Affymetrix 10K SNP array as previously described (Richardson et al. (2006) Cancer Cell 9, 121-132; Wang et al. (2004) Cancer Res. 64, 64-71). A portion of the SNP and gene expression data was reported previously (Matros et al. (2005) Breast Cancer Res. Treat. 91, 179-186; Lu et al. (2008) Breast Cancer Res. Treat. 108, 191-201; Richardson et al. (2006) Cancer Cell 9, 121-132; Wang et al. (2004) Cancer Res. 64, 64-71). Clinical and pathologic characteristics for each sample in the cohort are provided in Table 4.

B. Neoadjuvant Clinical Trials

The neoadjuvant Trial of Principle for breast cancer was conducted in European hospitals and coordinated at the Institut Jules Bordet. This trial is registered on the clinical trials site of the U.S. National Cancer Institute, which is available on the world wide web at http://clinicaltrials.gov/ct2/show/NCT00162812?term=N CT00162812&rank=1/. Single-agent epirubicin was given as neoadjuvant (preoperative) chemotherapy to 118 reportedly ER− cases. After central review, four of the cases were found to be ER+. Of the remaining 114 ER− cases, 87 cases were classified as HER2− on the basis of low v-erb-b2 erythroblastic leukemia viral oncogene homolog-2 (ERBB2 or HER2) module score (Desmedt et al. (2008) Clin. Cancer Res. 14, 5158-5165). Pretreatment core biopsy of the primary breast tumor was performed for diagnosis and RNA isolation. At completion of chemotherapy, pathologic response was determined by microscopic examination of the excised tumor and nodes. pCR was defined by the absence of residual invasive breast carcinoma in the breast and axillary nodes. This study was approved by the medical ethics committee of Institute Jules Bordet, and all women gave written informed consent before study entry. Gene expression data of U133plus 2 were generated from RNA of pretreatment core biopsies.

The trial of single-agent cisplatin (APP Pharmaceuticals) as neoadjuvant chemotherapy was given to women with ER−PR−HER2− breast cancer (Silver et al. (2010) J. Clin. Oncol. 28, 1145-1153). Gene expression array data from pretreatment biopsies was available for 24 cases (Silver et al. (2010) J. Clin. Oncol. 28, 1145-1153). Pathological response was determined by microscopy examination after chemotherapy as described above.

ROC curve analysis was performed to evaluate the mean level of combined LAPTM4B and YWHAZ expression, or the levels of each individual gene, for their capacity to predict pCR. The association of ranked gene expression levels was evaluated with pCR by determining the AUC estimated through the concordance index (Harrell et al. (1982) J. Am. Med. Assoc. 247, 2543-2546). The corresponding P values are from one-sided Wilcoxon's rank test (Pencina et al. (2004) Stat. Med. 23, 2109-2123). The ROC curves are plotted for prediction of pCR, so a curve below the midline in the low-right area of the graph indicates the ranked gene expression is associated with the absence of pCR.

C. Statistical Analysis of Gene Expression Arrays

Affymetrix U133plus2.0 array data from tumor samples of 115 cases were classified into those with distant recurrence within 36 months of diagnosis or those without distant recurrence after at least 36 months of follow-up. The 115 arrays were log-transformed and normalized, and invariable genes were removed by filtering with dChip software (P value <0.05). Differentially expressed genes were determined by PAM (Tibshirani et al. (2002) Proc. Natl. Acad. Sci. USA 99, 6567-6572) using the PAMR package implemented in R language (available on the world wide web at http://cran.r-project.org/web/packages/pamr/index.html). PAMR implements the shrunken nearest centroid method (Tibshirani et al. (2002) Proc. Natl. Acad. Sci. USA 99, 6567-6572). Genes were selected at a false discovery threshold that minimized a ten-fold cross-validation and test errors near the shrinkage parameter Δ=2. The PAM score indicates the degree of statistical association for expression of each gene and metastatic recurrence. Cox proportional hazard regression analysis (Tibshirani et al. (2002) Proc. Natl. Acad. Sci. USA 99, 6567-6572; Wang et al. (2005) Lancet 365, 671-679) was used to discover differentially expressed genes associated with time to recurrence.

D. Statistical Analysis in Independent Cohorts

Six independent gene expression array data sets (Chin et al. (2006) Cancer Cell 10, 529-541; Wang et al. (2005) Lancet 365, 671-679; van de Vijver et al. (2002) N. Engl. J. Med. 347, 1999-2009; Ivshina et al. (2006) Cancer Res. 66, 10292-10301; Sotiriou et al. (2006) J. Natl. Cancer Inst. 98, 262-272; Pawitan et al. (2005) Breast Cancer Res. 7, R953-R964) were pooled for analysis as previously described (van Vliet et al. (2008) BMC Genomics 9, 375). 8qEI was calculated as the mean expression value of the twelve 8q22 genes identified in PAM analysis, and this was scored in each of the six independent cohorts. An 8qEI level higher than the median was defined as 8qEI high and an 8qEI level lower than the median was defined as 8qEI low. The classification was performed in six cohorts separately and then combined into one data sheet with 1,348 samples. Of these, 1,130 had annotation for treatment and follow-up, 361 received adjuvant chemotherapy with or without hormonal therapy and 725 received no adjuvant hormonal or chemotherapy. Follow-up time was constrained to a maximum of 10 years with annotation for disease-free survival. Kaplan-Meier analyses were carried out with the survival package within the R statistical package. P values were derived with the Mantel-Cox log-rank test.

E. Microarray Data

Microarray data sets are deposited in the National Center for Biotechnology Information Gene Expression Omnibus database under the following accession numbers: gene expression data from the Dana-Farber/Harvard Cancer Center (DF/HCC) cases, GSE19615; SNP array data from 50 of the DF/HCC cases, GSE19594; gene expression data from the neoadjuvant epirubicin ‘Trial of Principle’, GSE16446; gene expression data from the neoadjuvant cisplatin trial, GSE18864.

F. Cell Lines, siRNA Transfection, and Gene Transfer

Breast cancer cell lines were maintained at 37° C. with 5% CO₂ in RPMI 1640 medium and/or DMEM medium supplemented with 10% FBS or other supplements as recommended by ATCC for each line. Human mammary epithelial cells (HMECs) carrying a dominant negative allele of p53 were cultured in DMEM/F12 containing 0.3% FBS, EGF (10 ng ml⁻¹ final), hydrocortisone (0.5 g ml⁻¹), cholera toxin (100 ng ml⁻¹), and insulin (10 μg ml⁻¹) (Zhao et al. (2005) Proc. Natl. Acad. Sci. U.S.A. 102, 18443-18448). HMEC cells are diploid at 8q22, express a low level of LAPTM4B and YWHAZ and are sensitive to doxorubicin. BT549 breast tumor cells have 8q22 gain and over-expression of 8q22 genes.

Gene-specific and negative control oligonucleotides (Stealth™ Select RNAi, Invitrogen) were transfected using the Lipofectamine™ RNAi MAX protocol from Invitrogen. Three gene-specific oligonucleotide siRNAs were tested for each gene (LAPTM4b siRNA1: 5′-CCCGUAUGAUGAUGCCACUGUGAAU-3′ and 3′-AUUCACAGUGGCAUCAUCAUACGGG-5′; LAPTM4b siRNA2: 5′-UCGGCGUCUGGUAUCUGAUCAUCAA-3′ and 3′-UUGAUGAUCAGAUACCAGACGCCGA-5′; LAPTM4b siRNA3: 5′-CAUCAAUGGUAGGAACUCCUCUGAU-3′ and 3′-AUCAGAGGAGUUCCUACCAUUGAUG-5′; LAPTM4b shRNA: Top strand, CACCGCTGATCCGGATCAGTATAACCGAAGTTATACTGATCCGGATCAGC, Bottom strand, AAAAGCTGATCCGGATCAGTATAACTTCGGTTATACTGATCCGGATCAGC; YWHAZ siRNA1: 5′-ACAGCACGCUAAUAAUGCAAUUACU-3′ and 3′-AGUAAUUGCAUUAUUAGCGUGCUGU-5′; YWHAZ siRNA2: 5′-GAGAGACAACUUGACAUUGUGGACA-3′ and 3′-UGUCCACAAUGUCAAGUUGUCUCUC-5′; and YWHAZ siRNA3: 5′-GGAUACCCAAGGAGACGAAGCUGAA-3′ and 3′-UUCAGCUUCGUCUCCUUGGGUAUCC-5′). Transfections were performed in triplicate and the experiment was repeated four times to estimate the effect and standard deviation from the mean. Because specific antibodies were not available for the 12 genes tested, the effectiveness of siRNA knockdown by each oligonucleotide was determined by qRT-PCR assay of mRNA levels according to methods described herein.

The full length LAPTM4B and YWHAZ cDNAs linked to an HA tag were cloned into the pWZL (Boehm et al. (2005) Mol. Cell. Biol. 25, 6464-6474) and pBabe vectors, respectively, and packaged using the Phoenix™ Retroviral System from Orbigen. pWZL-LAPTM4B HA and pBabe-YWHAZ-HA were introduced into HMEC cells by retroviral infection and stably selected with blasticidin and puromycin, respectively.

G. Cell Growth and Apoptosis Assays

Viable cell number was quantified using CellTiter 96® Aqueous One Solution Cell Proliferation Assay (Promega). Results are reported as optical density at 490 nm wavelength. Cells (4,000 well⁻¹) were seeded into 96-well plates in triplicate. siRNA transfections were performed 24 h after cell seeding. Chemotherapeutic drugs (daunorubicin, doxorubicin, paclitaxel, and cisplatin) were added 24 h after cell seeding or siRNA transfection. Cells were exposed to a series of concentrations of individual drugs for additional 48 h. Results are presented as the percentage of viable cells in drug-treated wells vs. media-treated control wells and plotted as a drug-dose dependent cell survive curve. The dose of drug causing a fifty percent reduction of viable cells (IC₅₀) determined drug sensitivity. Drug-induced apoptosis was evaluated 48 h after drug treatment (doxorubicin at 1 μM or cisplatin at 25 μM) following siRNA transfection by detection of active caspase 3 and cleaved poly(ADP-ribose) polymerase (PARP) by Western blot analysis using caspase 3- and PARP-specific antibodies (Cell Signaling Technology), respectively.

H. Cellular Distribution of Doxorubicin

Twenty-four h after transfection with specific siRNA or control oligos, cells were challenged with 0.5 μM doxorubicin in complete medium for 24 h. After 24 h, the drug was removed and cells were cultured in fresh drug-free medium and examined at 36 and 48 h time points. At each time point, cells were washed twice in PBS, and fixed for 5 min in 3.0% paraformaldehyde and permeabilized in 0.1% Triton X-100 for 5 min. Following washing with PBS, cells were mounted in Prolong Gold® anti-fade reagent with DAPI (Invitrogen). Subcellular localization of doxorubicin was viewed by the Rhodamine (Cy3) channel and nuclear staining was viewed by the DAPI channel on a Zeiss HAL100 fluorescence microscope.

I. qRT-PCR Assay

Total RNA was isolated using TriZol (Invitrogen). RT and PCR reactions were prepared using SYBR® GreenER™ Two-Step qRT-PCR Kit for iCycler (Invitrogen) and PCR amplification was performed using Bio-Rad ICycler iQ® Realtime PCR detection system. All reactions were performed in duplicate. The average C_{T} (threshold cycle) was calculated from duplicate reactions to analyze the mRNA levels as described further below. To adjust for different amount of cDNA in each reaction, averaged C_{T} values are normalized to C_{T} values for a control gene (GADPH). Relative mRNA levels=Δ (Δ C_{T}) (log 2 scale) or=2^{Δ(Δ CT)} (linear scale), with Δ C_{T}=C_{T}, target gene—C_{T}, GAPDH, and Δ (Δ C_{T})=Δ C_{T}, reference sample—Δ C_{T}, test sample.

J. Western Blotting

Tumor cell line lysates were fractionated in 14% SDS-PAGE, and electrophoretically transferred to a nitrocellulose membrane and probed with primary antibodies specific for β-actin (Sigma); antibodies specific for phospho-Histone H2A.X (Cell Signaling Technology), and HA-HRP specific antibody (12CA5) from Roche Applied Science), and HRP-conjugated secondary antibodies. The probed proteins were visualized using the ECL Western blotting detection system according to the manufacturer's instructions. The reference sample was RNA isolated from human mammary epithelial cells (HMECs). For the siRNA screening, the Δ (Δ C_{T}) values are relative to the control siRNA.

K. Fluorescence In Situ Hybridization (FISH)

Paraffin sections (5 μm) of four tissue microarrays, were hybridized to a BAC probe RP11-347C18 (Children's Hospital Oakland Research Institute, CA) to the 8q22 region labeled orange, and a probe to the chromosome 8 centromere (8 CEN, Vysis, Downers Groves, Ill.), and labeled with FITC (green). 8q22 copy number is reported as the average number of 8q22 signals per nucleus manually counted from 50 cells of each tumor analyzed. An average copy number of 3.5 obtained from interphase FISH was equivalent to ˜4 copies based on an average copy number of 1.7 observed for normal diploid cells in this assay. Average copy number >3.5 from FISH was considered as 8q22 gain.

L. Correlation of SNP or FISH Copy Number and Expression

Copy number was determined from SNP array data using dChip software. Copy gain was defined as copy number ≧4. Kendall correlation between copy number at each locus determined by SNP array and the expression of its closest gene neighbor by genomic location, and between 8q22 FISH copy number and expression level of all genes from chromosome 8 were computed with R package version 2.4.1.

M. Multivariate Cox Proportional Hazards Analysis

Univariate analysis was first performed for clinical prognostic factors including age. (years, continuous), axillary lymph node involvement (1=positive, 0=negative), tumor size (cm, continuous), tumor grade (1=grade III, 0=grade I or II), receipt of adjuvant chemotherapy (0=no, 1=yes), and 8q22 FISH (copy number >4 or <4). The most significant factors (p<0.05) were included for multivariate analysis.

### Example 2

### 8q22 Copy Number Aberrations and Overexpression of 8q22 Genes in Various Tumors

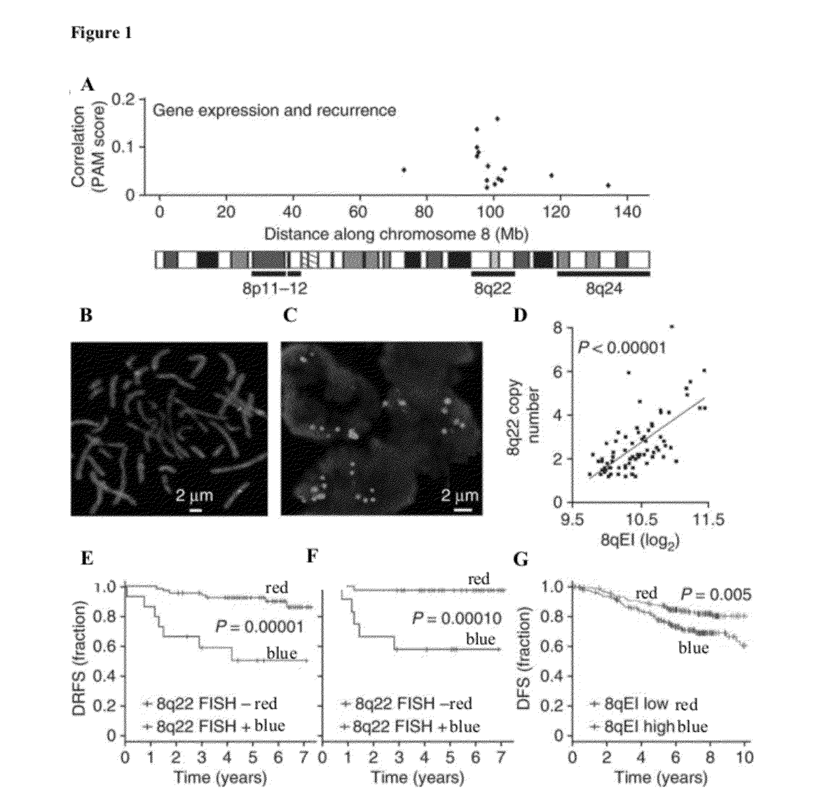

Gene expression profiles of 115 breast carcinomas from women diagnosed between 2000 and 2003 and treated according to current guidelines, including adjuvant chemotherapy if indicated, were analyzed. Predictive analysis of microarrays (PAM) (Tibshirani et al. (2002) Proc. Natl. Acad. Sci. USA 99, 6567-6572) analyses were performed and 114 probes were identified, encoding 75 known genes, differentially expressed between cases with early distant metastatic recurrence and cases without distant recurrence (Table 1). Fifteen percent of these probes, corresponding to 12 different genes, mapped to chromosome 8q22, the only chromosomal region with statistically significant enrichment (P<2.1×10⁻⁹) of probes associated with metastatic recurrence (FIG. 1A).

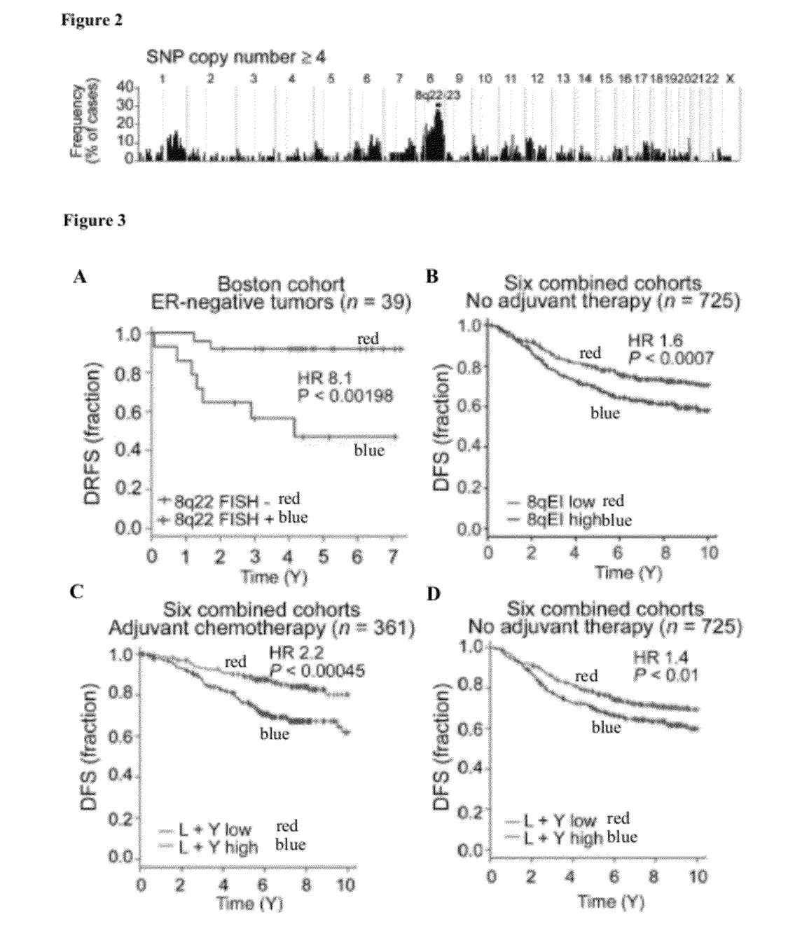

An alternative gene selection method based on Cox proportional hazard regression (Tibshirani et al. (2002) Proc. Natl. Acad. Sci. USA 99, 6567-6572; Wang et al. (2005) Lancet 365, 671-679) was also applied, which also showed differential overexpression of these 8q22 genes in tumors with distant recurrence. These genes included those encoding cyclin E2 (CCNE2) and metadherin (MTDH), which are reportedly associated with metastatic recurrence and poor prognosis of breast cancer (van't Veer et al. (2002) Nature 415, 530-536; Hu et al. (2009) Cancer Cell 15, 9-20). Coordinate overexpression of neighboring genes often reflects chromosomal amplification. Indeed, 8q22 amplification by single nucleotide polymorphism (SNP) array analysis in 50 breast cancers was observed (FIG. 2), and expression of the 8q22 genes correlated with DNA copy number (Table 1).

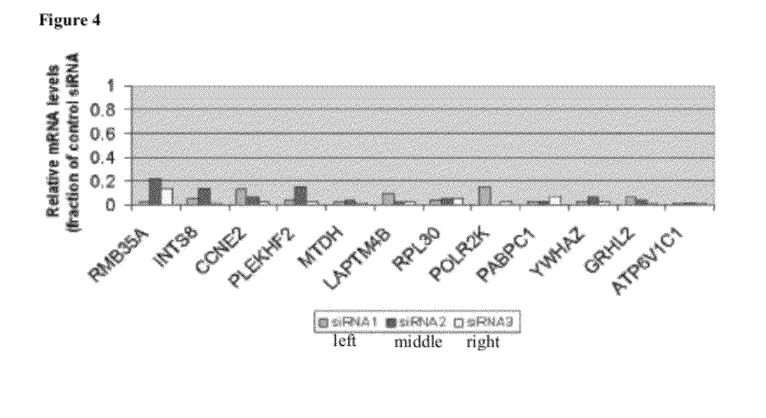

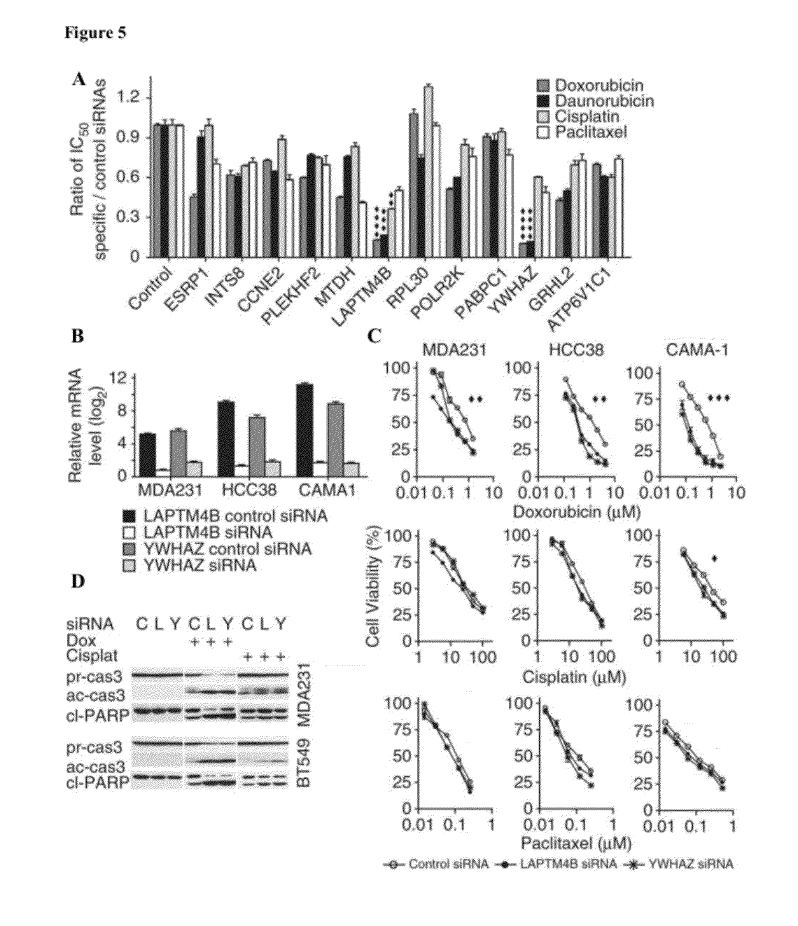

8q22 amplifications were confirmed by DNA interphase fluorescence in situ hybridization (FISH) and these were found in 21% of 85 breast cancers that could be scored by FISH (FIGS. 1B and 1C). The degree of copy gain correlated with average expression of the 12 recurrence-associated 8q22 genes (8q gene expression index, 8qEI) (FIG. 1D and Table 1). Kaplan-Meier analysis showed 8q22 amplification was associated with reduced metastasis-free survival in the entire cohort evaluated by FISH (FIG. 1E), in the estrogen receptor-negative (ER−) cases (FIG. 3A) and in the women who had received anthracycline-based adjuvant chemotherapy (FIG. 1F). In multivariate analysis, amplification of 8q22 was a strong independent prognostic factor for breast cancer recurrence (Table 2).

Meta-analysis of six independent cohorts annotated with treatment and outcome were used to validate the findings (Chin et al. (2006) Cancer Cell 10, 529-541; Wang et al. (2005) Lancet 365, 671-679; van de Vijver et al. (2002) N. Engl. J. Med. 347, 1999-2009; Ivshina et al. (2006) Cancer Res. 66, 10292-10301; Sotiriou et al. (2006) J. Natl. Cancer Inst. 98, 262-272; Pawitan et al. (2005) Breast Cancer Res. 7, R953-R964). Kaplan-Meier analysis showed a significant difference in disease-free survival between 8qEI low-expression and high-expression groups in either chemotherapy-treated (FIG. 1G) or untreated individuals (FIG. 3B). These results indicate that 8q22 amplification promotes overexpression of 8q22 genes in tumor tissue, which is associated with poor prognosis in untreated cases and shorter disease-free survival despite adjuvant chemotherapy.

### Example 3

### YWHAZ and LAPTM4B Gain-of-Function Significantly Increase Tumor Sensitivity to Anthracyclines

To determine whether 8q22 genes influence sensitivity to chemotherapy, the breast cancer cell line BT549 harboring chromosome 8q22 amplification was treated with siRNA specific for the 12 candidate genes (FIG. 4) and screened for alteration of sensitivity to chemotherapeutic drugs (FIG. 5A). Depletion of two genes in particular, YWHAZ and LAPTM4B, significantly increased the sensitivity to anthracyclines (FIG. 5A). YWHAZ codes for a known antiapoptotic protein, 14-3-3ζ (Niemantsverdriet et al. (2007) Oncogene 27, 1315-1319; Neal et al. (2009) Cancer Res. 69, 3425-3432). LAPTM4B encodes a recently described lysosomal protein called lysosomal associated protein transmembrane 4B, about which little is known with regard to breast cancer.

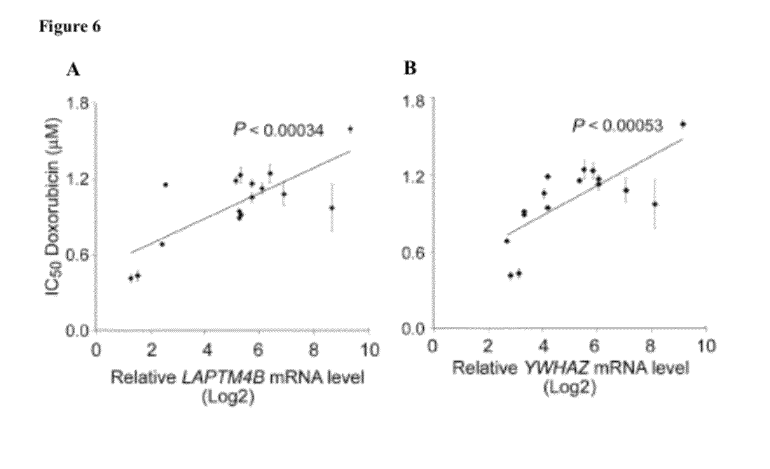

Sixteen breast cancer cell lines were examined and found to have a strong positive correlation between higher endogenous LAPTM4B mRNA level and higher half-maximal inhibitory concentration (IC₅₀) (relative resistance) to anthracyclines (P<0.00034, FIG. 6A) and a weaker or no correlation with IC₅₀ to cisplatin and paclitaxel (P=0.008 and P=0.4, respectively).

The expression of YWHAZ in cell lines also correlated with the IC₅₀ to doxorubicin (FIG. 6A). Specific knockdown of LAPTM4B and YWHAZ in three breast cancer cell lines, MDA-MB-231 (MDA231), HCC38 and CAMA-1 (FIG. 5B), increased sensitivity to the anthracyclines, doxorubicin (FIG. 5C) and daunorubicin, but had a weaker or no effect on sensitivity to cisplatin and paclitaxel (FIG. 5C). Knockdown of either LAPTM4B or YWHAZ considerably increased doxorubicin-induced apoptosis (FIG. 5D). Induction of apoptosis was less apparent in response to cisplatin treatment (FIG. 5D).

### Example 4

### YWHAZ and LAPTM4B Act Synergistically to Increase Tumor Sensitivity to Anthracyclines

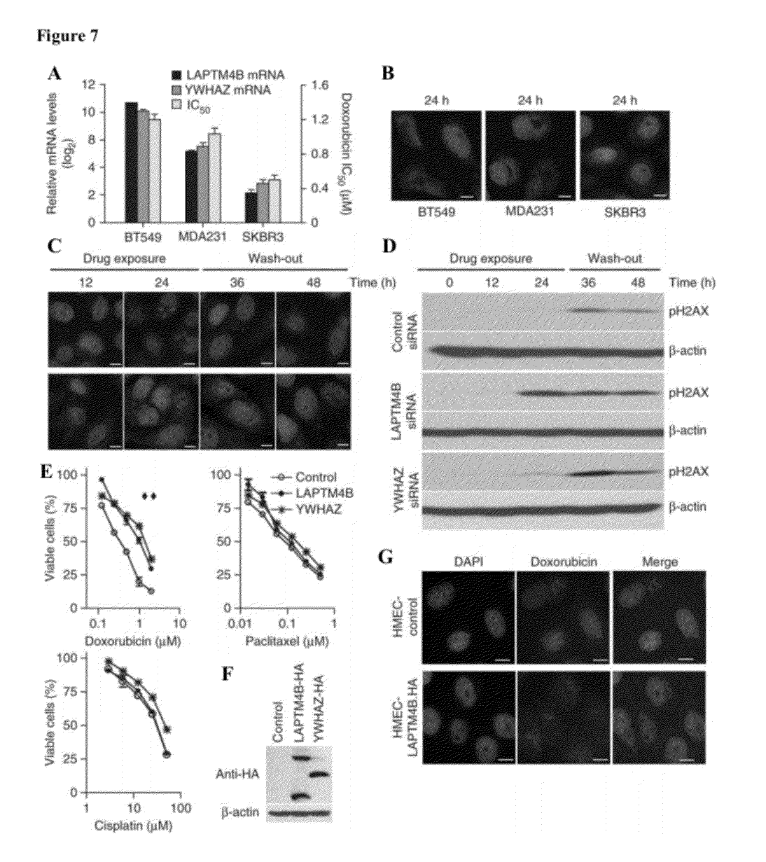

To investigate the mechanism of how these two genes were modulating sensitivity to anthracyclines, the intracellular localization of anthracyclines was tracked by following the autofluorescence of doxorubicin. LAPTM4B expression in cell lines correlated with both the IC₅₀ of doxorubicin (FIG. 7A) and the appearance of doxorubicin in the nucleus within 24 h (FIG. 7B). Knockdown of LAPTM4B by siRNA in MDA-MB-231 cells resulted in a substantial increase in nuclear localization of doxorubicin, detectable within 12 h to 24 h of treatment, maximal at 24 h to 36 h (12 h after withdrawal of drug) and sustained at 48 h (FIG. 7C). Knockdown of LAPTM4B in BT549 cells resulted in a similar increase in nuclear localization at 24 h. The decreased distribution of doxorubicin into the nucleus is associated with less phosphorylation of H2A histone family, member X (phospho-H2AX) (FIGS. 7C and 7D), a marker of DNA damage response, and consistent with reduced doxorubicin-induced apoptosis and increased IC₅₀ (FIG. 5). YWHAZ abundance correlated with the IC₅₀ of doxorubicin (FIG. 7A), and knockdown of YWHAZ slightly increased the phospho-H2AX levels in drug-treated cells (FIG. 7D). These results indicate that a mechanism of poorer outcome for women harboring breast cancers with 8q22 amplification is increased expression of LAPTM4B and interference with nuclear accumulation of anthracyclines, as well as the synergistic effect of YWHAZ on drug sensitivity through inhibition of apoptosis (Niemantsverdriet et al. (2007) Oncogene 27, 1315-1319; Neal et al. (2009) Cancer Res. 69, 3425-3432).

Hemagglutinin (HA)-tagged full-length LAPTM4B and YWHAZ were also introduced into partially transformed human mammary epithelial cells (HMECs) (Zhao et al. (2005) Proc. Natl. Acad. Sci. USA 102, 18443-18448). Expression of either exogenous LAPTM4B or exogenous YWHAZ increased the 1050 of doxorubicin (270% increase, P<0.002 or 394% increase, P=0.0001, respectively) but had no significant effect on sensitivity to paclitaxel or cisplatin (FIGS. 7E and 7F). The LAPTM4B-induced decrease in drug sensitivity parallels and is consistent with delayed appearance of anthracycline in the nucleus of LAPTM4B-overexpressing HMECs (FIG. 7G).

### Example 5

### YWHAZ and LAPTM4B Gain-of-Function is Associated with Poorer Outcomes of Adjuvant Chemotherapy Treatment

Kaplan-Meier analysis of women treated with adjuvant chemotherapy showed that the expression of YWHAZ and LAPTM4B above median level was associated with shorter disease-free survival (FIG. 3C). The association of higher expression levels of YWHAZ and LAPTM4B with poor outcome after adjuvant chemotherapy is consistent with either a prognostic effect or a role of these two genes in chemotherapy resistance.

### Example 6

### YWHAZ and LAPTM4B Gain-of-Function is Associated with Poorer Outcomes of Neoadjuvant Chemotherapy Treatment

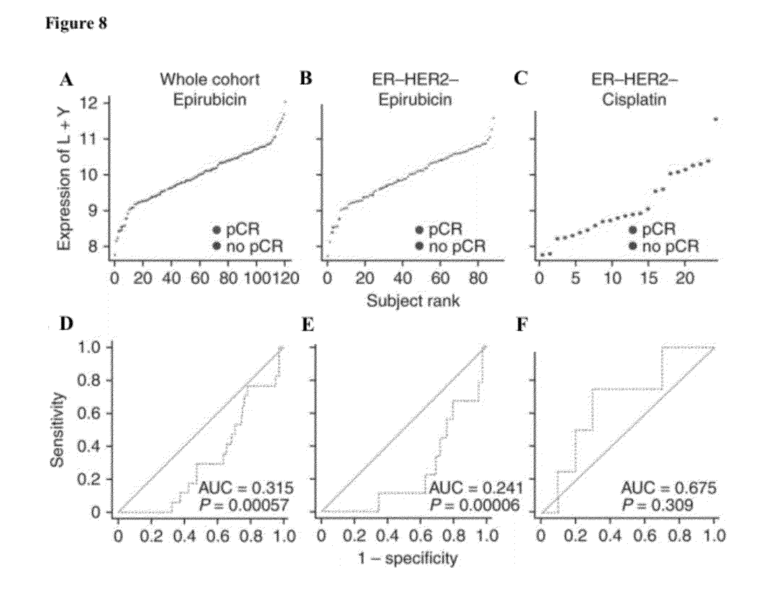

LAPTM4B and YWHAZ were tested for their association with response to anthracyclines in a neoadjuvant (preoperative) treatment trial of epirubicin monotherapy and with response to cisplatin in a neoadjuvant trial of cisplatin monotherapy (FIG. 8). The average of LAPTM4B and YWHAZ expression levels were evaluated from expression array data of pretreatment tumor biopsies for their association with pathologic complete response (pCR) to epirubicin. The two gene expression levels showed a coherent pattern with higher levels of expression associated with the absence of pCR (and the presence of residual disease) after epirubicin treatment (FIGS. 8A and 8B). The capability of the two genes to predict pCR was evaluated by measuring the area under receiver operator characteristic (ROC) curve (AUC) (Zweig et al. (1993) Clin. Chem. 39, 561-577; Farmer et al. (2009) Nat. Med. 15, 68-74), which showed that higher expression of the two genes is associated with the absence of pCR after anthracycline chemotherapy in the cohort of 118 breast tumors (AUC 0.315, P<0.00058, FIG. 8D). The association is more significant in 87 ER− tumors that were also negative for overexpression of v-erb-b2 erythroblastic leukemia viral oncogene homolog-2(HER2−) tumors (AUC 0.241, P<0.000062, FIG. 8E).

When the two genes were analyzed separately, both genes were predictive of poor response in the whole cohort and in the ER−HER2− subset, but only LAPTM4B level was predictive in the ER−HER2+ subset (Table 3). In contrast, the expression of the two genes was not associated with treatment response to cisplatin monotherapy in a separate neoadjuvant clinical trial in tumors negative for ER, progesterone receptor (PR) and HER2 (ER−PR−HER2−) (Silver et al. (2010) J. Clin. Oncol. 28, 1145-1153) (AUC 0.675, P>0.3, FIGS. 8C and 8F and Table 3). Although MTDH, one of the twelve 8q22 genes, has been reported to induce chemoresistance to a broad spectrum of drugs in experimental models (Hu et al. (2009) Cancer Cell 15, 9-20), its expression was not predictive for pCR in either the epirubicin or the cisplatin trial.

A third single-agent neoadjuvant therapy trial of predominantly ER+ tumors treated with docetaxel was evaluated (Chang et al. (2003) Lancet 362, 362-369) and it was found that higher levels of expression of LAPTM4B and YWHAZ were not associated with an inferior clinical response to therapy. These results support the notion that LAPTM4B and YWHAZ overexpression is associated preferentially with poor response to anthracyclines.

LAPTM4B is similar to its family member LAPTM4A, which promotes selective resistance to anthracyclines but not cisplatin in Saccharomyces cerevisiae (Hogue et al., (1999) J. Biol. Chem. 274, 12877-12882). The results presented herein indicate that LAPTM4B acts on anthracycline trafficking by reducing drug entry into the nucleus and decreasing drug-induced DNA damage. Higher YWHAZ expression protects cells from drug-induced apoptosis. Because these genes reside in proximity on chromosome 8q, amplification produces coordinated upregulation of their various functions, together resulting in preferential resistance to anthracyclines. The results from the three clinical trials support this contention and indicate that clinical options for the treatment of primary breast cancers might depend upon the status of 8q22 amplification and overexpression of LAPTM4B and YWHAZ in tumors. Anthracyclines are believed to be a reasonable treatment in tumors without 8q22 amplification, and alternatives might be selected for those whose cancers harbor amplification.

### Example 7

### Materials and Methods for Examples 8-12

A. Reagents

Doxorubicin and FITC-Dextran were obtained from Sigma Corp. The irreversible cysteine protease inhibitor trans-epoxysuccinyl-L-leucylamindo-3-methylbutane ethyl ester (EST), the reversible aspartic protease inhibitor pepstatin A, and the reversible caspase inhibitor z-Val-Ala-Asp-fluoromethylketone (z-VAD-fmk) were obtained from Calbiochem Corp. Solutions of EST, z-VAD-fmk and Pepstatin A were prepared at a concentration of 50 μM, 25 mM and 100 μM, respectively.

B. Cell Lines, siRNA Transfection and Gene Transfer

Breast cell lines BT549 and MDA-MB-468 were obtained from American Type Culture Collection. Tert-immortalized human mammary epithelial cells (HMEC) were from Clontech Inc. BT549 cancer cells have amplification and overexpression of LAPTM4B (Neve et al. (2006) Cancer Cell, 10, 515-527) and were used for siRNA knockdown experiments. MDA-MB-468 (MDA468) cancer cells have normal DNA copy number and expression of LAPTM4B (Neve et al. (2006) Cancer Cell, 10, 515-527) and were chosen for LAPTM4B-transfection for overexpression and tumor xenograft experiments. Cell culture condition, siRNA transfection, expression construct carrying LAPTM4B cDNA, and stable transfer of this construct into MDA468 were performed as described in (Li et al. (2010) Nat. Med., 16, 214-218).

C. Analysis of Lysosomal Membrane Permeabilization (LMP)

Control and LAPTM4B expression-modified cells were incubated with FITC-labeled dextrans of different molecular weights from 4, 40, and 70 KDa (Sigma Corp.) for 12 hours, followed by wash out of excess dextran with culture medium. The cells were examined on a Zeiss HAL100 fluorescence microscope and photographed with a Zeiss camera.

D. Immunocytochemical Detection of LAPTM4B Localization in Cells

Cells plated on cover-slips were treated with Lyso Tracker DND-99 (Invitrogen) for 1 hour, fixed in 4% formaldehyde and rinsed in PBS. Fixed cells were permeabilized with 0.2% Triton X-100 in PBS for 6 min and washed with PBS containing 5% BSA. The cells were incubated with 1:100 diluted polyclonal rabbit anti-human antibodies to six His (Cell Signaling) for 1 hour in a humidifier at room temperature. After rinsing in the incubation buffer (2×5 min), the specimens were exposed to 1:5000 diluted goat anti-rabbit IgG-FITC (Abcam) for 1 hour at room temperature, washed and mounted. Subcellular localization of LAPTM4B and lysosomes were viewed by fluorescence microscopy.

E. Cellular Distribution of Doxorubicin

Forty-eight hours after transfection with specific siRNA or scramble control oligonucleotide, cells were treated with 0.1 μM doxorubicin in complete medium for 24 hours. After 24 hours of drug exposure, the drug was removed and cells were cultured in fresh drug-free medium and examined 24 hours later. Cells were washed, fixed and stained with DAPI as described in Li et al. (2010) Nat. Med., 16, 214-218. Subcellular localization of doxorubicin autofluorescence (excited wave: 480 nm; emitted wave: 560-620 nm) and lysosomes indicated by LysoTracker DND-26 (excited wave: 504 nm; emitted wave: 509-540 nm) was examined on a Leica SP5X Laser Scanning Confocal Microscope with a white light laser.

F. Cell Growth and Apoptosis Assays

The doxorubicin treatment, cell viability and proliferation assay, and evaluation of apoptosis by immunoblot were performed as described previously in Li et al. (2010) Nat. Med., 16, 214-218.

G. Cell Growth in Low Nutrient, Hypoxic, or Acidic Conditions

BT549 cells were plated in 96 wells plates of nutrient rich DMEM medium (10% fetal calf serum (FCS)), low serum DMEM medium (1% FCS), or glucose-free DMEM medium (Invitrogen, Inc.). After 1, 2, 3, and 4 days incubation, cell viability was determined as described in Li et al. (2010) Nat. Med., 16, 214-218. Acidified medium was prepared using HCl to adjust pH and measured using a pH meter. Cell viability was determined after 72 hours incubation in acidic medium. For hypoxia-induced stress, cells were transferred to a hypoxia chamber (Coy laboratory, Grass Lake, Mich.) with a humidified atmosphere of 1% O₂ and 5% CO₂ balanced with N₂ and incubated at 37° C. for 5 days. Cell viability was measured as described in Li et al. (2010) Nat. Med., 16, 214-218. The data were presented as mean±SD of three different experiments.

H. Tumorigenicity Assay

Two groups of mice were injected with LAPTM4B expression-manipulated MDA468 cells or the control counterpart cells into the inguinal mammary fat pads. Mice were routinely monitored for health and tumor size. Mice were sacrificed when tumors reached 2 centimeters. Tumor growth rates were measured and compared between LAPTM4B-manipulated and control tumors. LAPTM4B expression level and the autophagosome marker LC3 were measured by immunoblot in tumor lysates prepared from xenograft tumors.

I. qRT-PCR Assay

qRT-PCR assays were performed and relative mRNA level was calculated as described in Li et al. (2010) Nat. Med., 16, 214-218.

J. Extraction of Cytosol

Cytosolic extraction was performed using the detergent digitonin which permeabilizes cholesterol-rich membranes such as plasma membrane, leaving the cholesterol-poor membranes of lysosomes and mitochondria more or less intact. Cells were exposed to an extraction buffer (250 mM sucrose, 20 mM Hepes, 10 mM KCl, 1.5 mM MgCl2, 1 mM EDTA, 1 mM EGTA, and 1 mM Pefabloc, pH 7.5) containing 25 μg/ml digitonin for 20 min on ice, inverting the tubes every 2 min. The extraction buffer was collected and proteins were precipitated by addition of 5% trichloric acid on ice for 10 min and centrifuged at ˜20,800×g for 15 min to obtain the protein pellet. The pellet was dissolved in lysis buffer.

K. Immunoblotting

Tumor cell line lysates or cytosols were fractionated in 7, 12 or 14% SDS-PAGE, electrophoretically transferred to a nitrocellulose membrane and probed with primary antibodies (monoclonal Anti-β-Actin antibody (Sigma), anti-Bid (BD PharMingen), anti-caspase 3, anti-H is, anti-LC3 and anti-PARP antibodies (Cell Signaling Technology), and anti-cathepsin B, anti-cathepsin D, anti-p62, HRP-conjugated secondary antibodies and FITC-conjugated secondary antibodies (Abcam). The probed proteins were visualized using the ECL Western blotting detection system from Pierce according to the manufacturer's instructions. Band densities were quantified on a AlphaImager HP imaging system (Cell Biosciences).

L. Statistical Analysis

Differences in cell viability, tumor size, and ratios of LC3II/LC3I, or of p62/actin between control and testing groups were evaluated by Student t-test and p-values derived from this test were used to determine the significance of the difference.

### Example 8

### LAPTM4B Localizes to Lysosomes in Mammary Epithelial Cells

Chemoresistance is often an acquired characteristic of recurrent tumors either induced or selected by exposure to a drug. Alternatively, de novo drug resistance can occur due to intrinsic features of the primary tumor which are presumably selected by contributing a proliferative or survival advantage to tumor cells. The possible mechanisms of chemoresistance are complicated and may include altered drug uptake, intracellular distribution, efflux and turnover. Lysosomal retention of drugs is associated with drug resistance and lysosomal concentration of anthracyclines is thought to increase drug efflux and decrease drug nuclear localization, thereby preventing effective chemotherapy-induced DNA damage (Larsen et al. (2000) Pharmacol. Ther. 85, 217-229).

Lysosomes are spherical organelles that contain many hydrolytic enzymes, such as proteases, nucleases and lipases. Through release of lysosomal contents, lysosomal-membrane permeabilization (LMP) can pose a threat to cellular homeostasis and is a recognized trigger of cell death (Johansson et al. (2010) Apoptosis 15, 527-540). For example, a selective and limited release of lysosomal cathepsins B or D may activate the mitochondrial cell death pathway by either generating reactive oxygen species and lipid mediators or triggering mitochondrial outer membrane permeabilization. Cathepsin B is major mediator of apoptotic pathways activating the pro-apoptotic protein Bid by truncation. Truncated Bid (tBid) translocates to mitochondria to active the caspase cascade and promotes apoptosis. Cathepsin D is an aspartate protease that directly activates caspase (Baumgartner et al. (2007) Am. J. Physiol. Gastrointest. Liver Physiol., 293, G296-307; Conus et al. (2008) J. Exp. Med, 205, 685-698). The current knowledge of the molecules on lysosomal membranes critical for affecting or regulating these various lysosomal functions is limited.

Autophagy is a conserved lysosome-mediated intracellular trafficking pathway that degrades and recycles intracellular components (Burman et al. (2010) Semin. Immunopathol., 32, 397-413). Autophagy is also a homeostatic mechanism that regulates metabolism and energy production and may be up regulated in response to a variety of cell stresses (Burman et al. (2010) Semin. Immunopathol., 32, 397-413; He et al. (2009) Annu. Rev. Genet., 43, 67-93; Klionsky, D. J. (2005) J. Cell Sci., 118, 7-18; Rabinowitz et al., (2010) Science, 330, 1344-1348). As cancers develop, the process of autophagy (autophagy flux) may be up-regulated to support tumor cell survival and allow tumors to adapt to these stresses (Jin et al. (2007) Autophagy, 3, 28-31; Degenhardt et. al. (2006) Cancer Cell, 10, 51-64). Conversely, too much autophagy may catabolize essential components resulting in autophagic cell death (Scarlatti et al. (2009) Cell Death Differ., 16, 12-20; Eisenberg-Lerner et al. (2009) Cell Death Differ., 16, 966-975). Autophagy is regulated by a signaling cascade involving mammalian target of rapamycin (mTOR) pathway inhibition, the autophagy proteins (Atgs), and two ubiquitin-like conjugation systems (Klionsky et al. (2003) Dev. Cell, 5, 539-545; Yorimitsu et al. (2005) Cell Death Differ., 12 Suppl 2, 1542-1552; Wullschleger et al. (2006) Cell, 124, 471-484; Hanada et al. (2007) J. Biol. Chem., 282, 37298-37302; Sou et al. (2008) Mol. Biol. Cell, 19, 4762-4775). During autophagy initiation, a portion of the cytosol is surrounded by a cisternal membrane, the phagophore (Seglen et al. (1990) Semin. Cell Biol., 1, 441-448), that closes to form a double-membraned vesicle, the autophagosome (Juhasz et al. (2006) PLoS Biol., 4, e36). During autophagosome formation, cytosolic LC3 is cleaved by a protease and then conjugated to phosphatidylethanolamine to form autophagosomal membrane-associated LC3_{II} (Kabeya et al. (2000) EMBO J., 19, 5720-5728); the level of LC3_{II} correlates with the number of autophagosomes (Klionsky et al. (2007) Autophagy, 3, 181-206; Klionsky et al. (2008) Autophagy, 4, 151-175). After their formation, autophagosomes fuse with endosomes to form amphisomes (Razi et al. (2009) J. Cell Biol., 185, 305-321; Stromhaug et al. (1993) Biochem. J. 291 (Pt 1), 115-121) and then with lysosomes to form autolysosomes where sequestered material is degraded (Burman et al. (2010) Semin. Immunopathol., 32, 397-413; Dunn, W. A., Jr. (1990) J. Cell Biol., 110, 1935-1945; Eskelinen, E. L. (2005) Autophagy, 1, 1-10; Mehrpour et al. (2010) Cell Res., 20, 748-762). Blockade of autophagosome maturation and fusion with the lysosomal compartment results in accumulation of autophagosomes and interruption in the flux of material through the autophagic pathway (Rubinsztein et al. (2009) Autophagy, 5, 585-589). Autophagic flux can be monitored by measuring the level of substrates normally degraded by autophagy such as p62/SQSTM1 (Klionsky et al. (2008) Autophagy, 4, 151-175). The p62 protein is a ubiquitin-binding scaffold protein that binds directly to LC3 and is itself degraded in autolysosomes (Pankiv et al. (2007) J. Biol. Chem., 282, 24131-24145). Inhibition of autophagic degradation results in an increase in p62 levels and increased autophagy flux is indicated by decreased p62 levels (Bjorkoy et al. (2009) Methods Enzymol., 452, 181-197). Many molecules have been implicated in these later stages of autophagosome maturation (Mehrpour et al. (2010) Cell Res., 20, 748-762).

Lysosomal-associated protein transmembrane-4 beta (LAPTM4B) is a putative novel oncogene (Shao et al. (2003) Oncogene, 22, 5060-5069; Kasper et al. (2005) Cancer Lett., 224, 93-103) frequently amplified and overexpressed in primary treatment-naive breast cancers (Li et al. (2010) Nat. Med., 16, 214-218). Like other LAPTM family members, LAPTM4B protein has a lysosome localization motif (Shao et al. (2003) Oncogene, 22, 5060-5069) and co-localizes with markers of late endosomes and lysosomes (Vergarajauregui et al. (2010) J. Cell Sci., 124, 459-468). Increased expression of LAPTM4B has been demonstrated in hepatocellular carcinoma (HCC), and lung, ovarian, uterine, and gastric cancers (Kasper et al. (2005) Cancer Lett., 224, 93-103; Liu et al. (2007) Ann. Oncol., 18, 311-316; Yang et al. (2010) J. Cancer Res. Clin. Oncol., 136, 275-281; Meng et al. (2010), Int. Gynecol. Cancer, 20, 745-750). Overexpression of the LAPTM4B-35 isoform in hepatocellular carcinoma cell lines promotes apoptosis resistance in vitro and growth and metastasis of HCC xenografts in mice (Yang et al. (2010) Cancer Lett., 294, 236-244). Overexpression of LAPTM4B in primary breast tumors is associated with resistance to chemotherapy, specifically anthracyclines, and thereby can serve as a predictive biomarker for distant recurrence in patients treated with adjuvant chemotherapy (Li et al. (2010) Nat. Med., 16, 214-218). By sequestering drug in cytoplasmic compartment and enhancing efflux of drugs from cancer cells, overexpression of LAPTM4B decreases nuclear localization of drug and drug-induced DNA damage and thereby reduces drug effectiveness (Li et al. (2010) Nat. Med., 16, 214-218; Li et al. (2010) Oncogene, 29, 5785-5795). It is described herein the potential mechanisms by which LAPTM4B promotes tumor growth and survival in treatment-naive cancers. Specifically, high expression of LAPTM4B inhibits lysosome-mediated death pathways, promotes autophagy, and leads to stress tolerance, thereby enhancing tumor cell growth and survival. LAPTM4B may be an important new therapeutic target for inhibiting cancer growth or sensitizing tumors to chemotherapy.

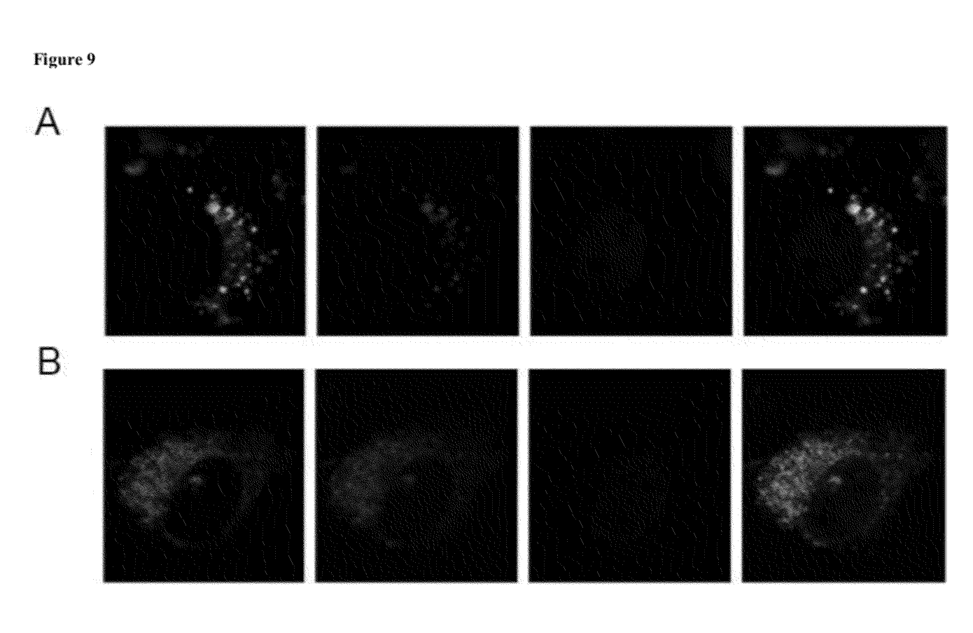

LAPTM4B is a member of a family of proteins which contain a lysosome targeting motif at the C-terminus (Shao et al. (2003) Oncogene, 22, 5060-5069). To determine lysosome localization in mammary epithelial cells, HMEC cells stably expressing exogenous six-His epitope tagged LAPTM4B were analyzed by immunofluorescence microscopy and expression of His-LAPTM4B in the lysosomal compartment was determined using the lysosome-marker Lysotracker DND-99 (FIG. 9A). Lysosomes are known to play a role in drug trafficking and overexpression of LAPTM4B is associated with cytoplasmic retention of the anthracycline drug, doxorubicin (Li et al. (2010) Nat. Med., 16, 214-218). To further investigate the intracellular cytoplasmic distribution of doxorubicin in cells that over express LAPTM4B, immunofluorescence confocal microscopy was performed on BT549 cells treated with doxorubicin. As shown in FIG. 9B, doxorubicin auto-fluorescence co-localizes with the lysosome marker, Lysotracker green DND-26. This finding indicates that over-expression of LAPTM4B may alter lysosomal function in a way that leads to increased lysosomal uptake and retention of anthracycline drugs.

### Example 9

### Down Regulation of LAPTM4B Triggers Lysosomal Membrane Permeabilization (LMP)

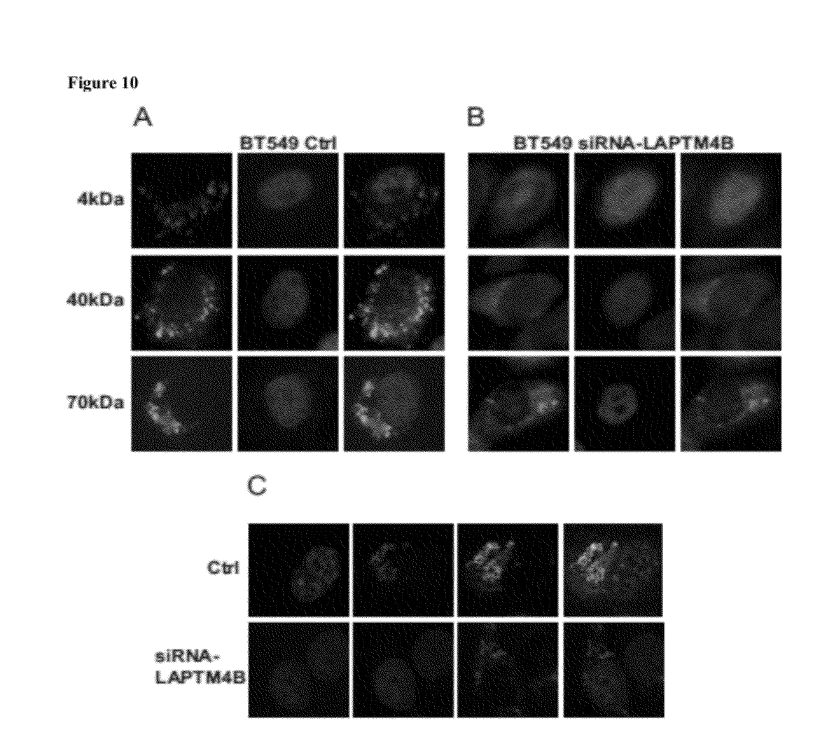

Lysosomes are intracellular organelles that contain acid hydrolase enzymes that digest macromolecules from phagocytosis, endocytosis, or autophagy. The lysosomal membrane protects the cytosol from exposure to degradative enzymes. To understand how LAPTM4B might modulate lysosome function, the relationship between LAPTM4B expression and lysosome membrane permeability was investigated. LMP was evaluated by measuring endocytic uptake and release of a non-digestible fluid-phase substrate, fluorescein isothiocyanate (FITC)-dextran. In BT549 breast cancer cells transfected with a scramble control vector, the dextran particles were taken up and retained in lysosomes (FIG. 10A). Knockdown of LAPTM4B expression by siRNA resulted in a diffuse release of the 4 kDa and 40 kDa FITC-dextran molecules throughout the cell (FIG. 10B). There is not global destabilization of lysosomes upon LAPTM4B knockdown as the larger molecular weight 70 kDa FITC-dextrans were mostly retained in cytoplasmic vesicles (FIG. 10B, lower panels). These results demonstrate that LAPTM4B exerts lysosome-stabilizing properties to retain low and intermediate molecular weight macromolecules. Doxorubicin is small molecule with a molecular weight of approximately 580 Dalton. As shown in FIG. 10C, down-regulation of LAPTM4B in BT549 cells led to a similar release of doxorubicin from lysosomes, resulting in much of the drug being relocated into the nucleus.

### Example 10

### Knockdown of LAPTM4B by siRNA Provokes Lysosomal-Mediated Programmed Cell Death Through Induction of Cathepsin Release

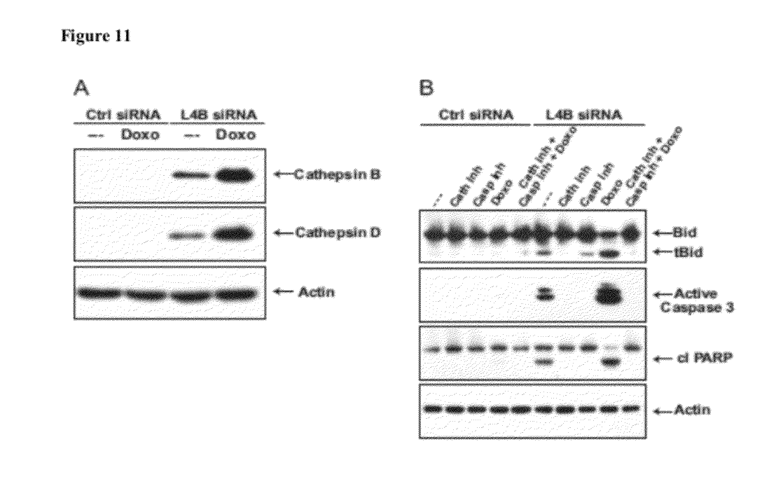

The maintenance of lysosomal membrane integrity is important for cell survival. LMP leads to cathepsin release followed by caspase activation. This process initiates apoptosis which in turn triggers further lysosomal destabilization to induce lysosomal-mediated cell death in a positive feed back loop (Johansson et al. (2010) Apoptosis 15, 527-540). To determine if LAPTM4B knockdown led to cathepsin released from lysosomes, immunoblot analysis for cathepsins was performed on cell cytosol fractions of BT549 tumor cells after transfection with control or LAPTM4B-specific siRNA. Depletion of LAPTM4B resulted in appearance of cathepsin B and cathepsin D in the cytosol of cells and cathepsin release was more pronounced when the cells were treated with doxorubicin (FIG. 11A). It was next tested whether cathepsin release after LAPTM4B depletion led to initiation of apoptosis. As shown in FIG. 11B, knockdown of LAPTM4B alone resulted in Bid truncation, caspase 3 activation and PARP cleavage, an effect that was more marked after exposure to doxorubicin. Pan inhibitors of cathepsins (EST and pepstatin A) protected BT549 cells from LAPTM4B knockdown-induced Bid truncation and apoptosis. The pan-caspase inhibitor z-VAD-FMK was sufficient to abrogate caspase 3 activation but did not prevent Bid truncation. These results indicate that a certain level of LAPTM4B is required to prevent lysosome-mediated initiation of apoptosis. This requirement is more notable in the setting of additional cellular insult, such as exposure to chemotherapy agents.

### Example 11

### LAPTM4B Over-Expression Promotes Autophagy

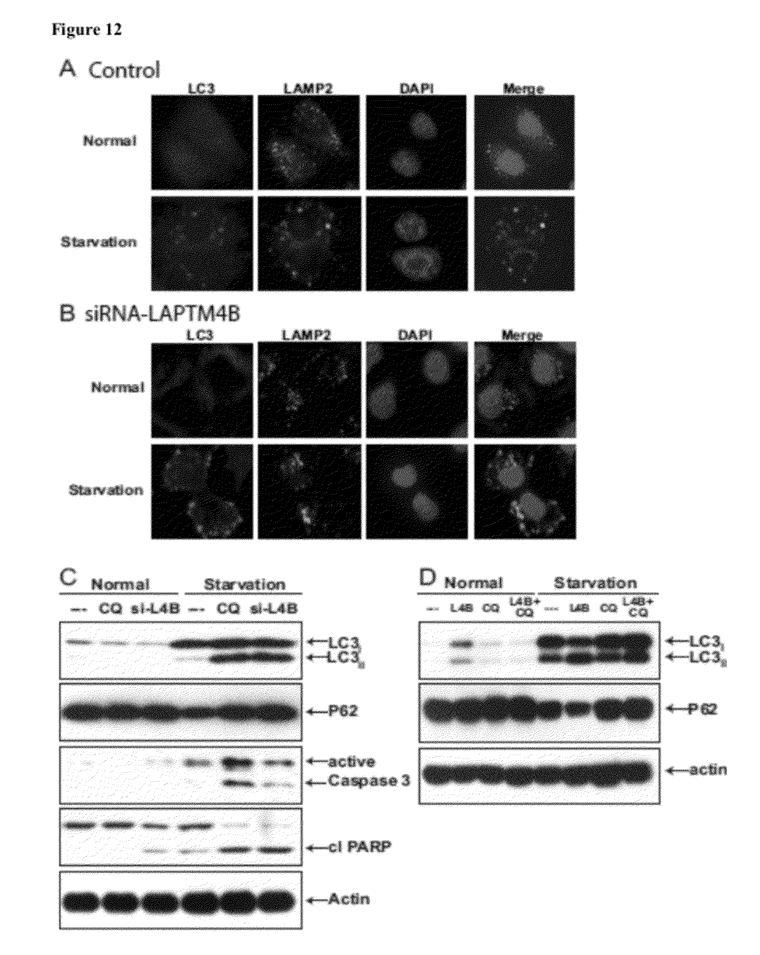

Autophagy plays a critical survival role by supporting energy requirements and sustaining viability under adverse conditions and may also promote resistance to chemotherapy induced genotoxic stress. To determine if modulation of LAPTM4B expression has an effect on autophagy, markers of autophagosome maturation and flux were examined during starvation-induced autophagy in BT549 breast cancer cells with or without depletion of LAPTM4B. In parental BT549 cells (with inherent overexpression of LAPTM4B), autophagy was induced by starvation as indicated by the appearance of punctuate LC3 staining in the cytoplasm. Many of the LC3-puncta co-localized with the lysosome marker LAMP2 indicating normal autophagosome maturation and fusion to lysosomes (FIG. 12A). In contrast, in cells in which LAPTM4B has been depleted by siRNA, starvation resulted in marked cytoplasmic accumulation of enlarged LC3-positive autophagosomes which were not co-localized with LAMP2-positivelysosomes (FIG. 12B). This indicates that depletion of LAPTM4B results in a block in autophagosome-lysosome fusion and blocked autolysosome formation.

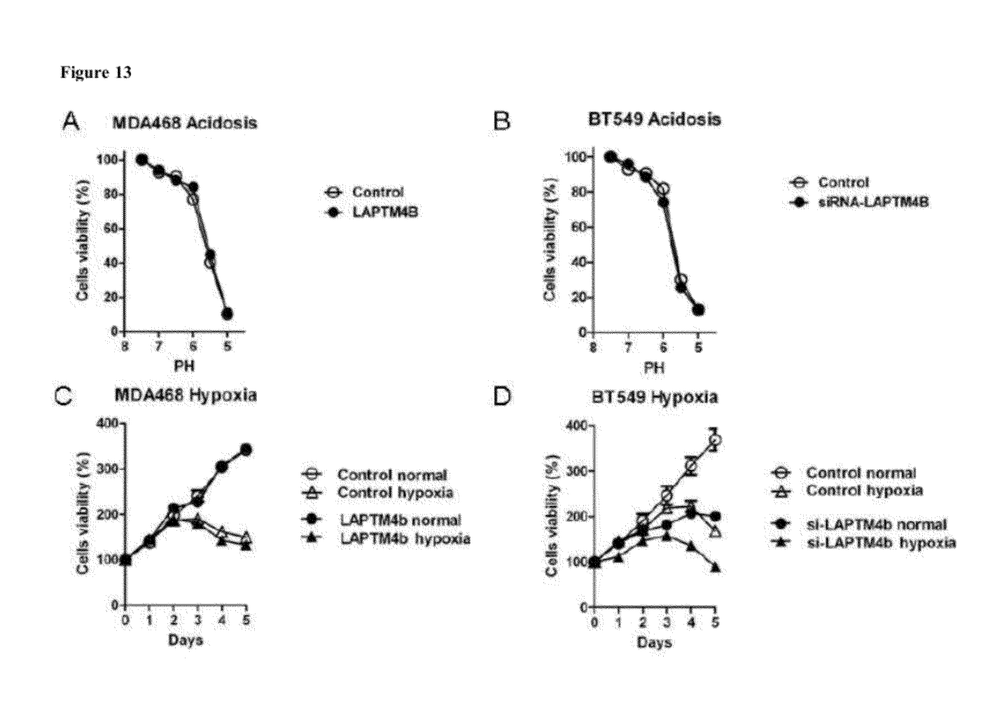

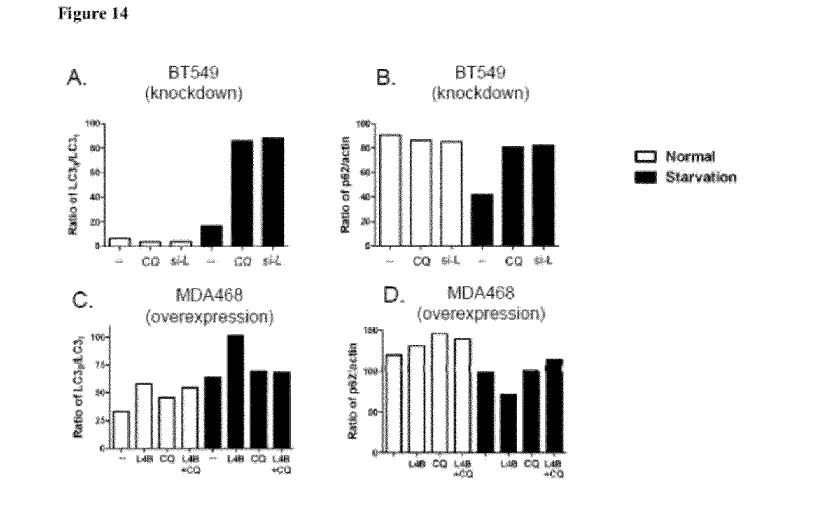

Autophagy was also analyzed in these cells by immunoblotting for LC3_{II} and p62. siRNA depletion of LAPTM4B had no effect on the level of LC3_{II} in BT549 cells grown in nutrient rich medium, but resulted in significant increase in the level of LC3_{II} when cells were stressed by serum starvation (FIG. 12C and FIG. 13A). LC3II is an indicator of autophagosome number and levels can increase due to increased autophagosome formation or from a block in autophagosome maturation or autolysosomal degradation or both (Rubinsztein et al. (2009) Autophagy, 5, 585-589). To determine if autophagy flux was increased or blocked, levels of the autophagy substrate, p62, were measured. In control cells, serum starvation increased autophagy flux as indicated by lower p62 levels in the serum starved cells compared to cells grown in nutrient rich medium. However, in cells depleted of LAPTM4B by siRNA, there was no change in the level of p62 after serum starvation, indicating a block in starvation-induced autophagy flux (FIG. 12C and FIG. 14B). The effect of knockdown of LAPTM4B on LC3_{II} and p62 levels was similar to what was observed in serum starved cells treated with chloroquine (CQ), a lysosomotropic agent that raises the lysosomal pH and inhibits lysosome-autophagosome fusion and blocks autolysosome degradation. In conjunction with the immunofluorescence findings, these results indicate that LAPTM4B is required for the later stages of autophagy maturation in which autophagosomes are fused with lysosomes to form autolysosomes.

Abortive accumulation of autophagasomes might act as a cell death messenger to trigger caspase dependent or independent cell death. Accordingly, an increase in cell death was observed, as indicated by caspase 3 activation and cleaved PARP in LAPTM4B-depleted cells with starvation-induced aggregation of autophagasomes (FIG. 12C). Although down-regulation of LAPTM4B alone may trigger LMP and to a lesser extent caspase activation, the combination of LMP and blocked autophagy results in more pronounced caspase activation and cell death. In a complimentary experiment, stable over expression of LAPTM4B in MDA468 cells with inherently low levels of LAPTM4B, resulted in greater starvation-induced autophagy as indicated by higher levels of LC3_{II} and lower levels of p62 (FIG. 12D and FIGS. 14C-14D). This effect was blocked by the autolysosome inhibitor chloroquine. These results are consistent with the notion that over-expression of LAPTM4B promotes increased autophagy flux in cancer cells exposed to metabolic stress.

### Example 12

### Over Expression of LAPTM4B Promotes Tumor Cell Survival and Resistance to Apoptosis Induced by Low Serum Concentration or Glucose Deprivation

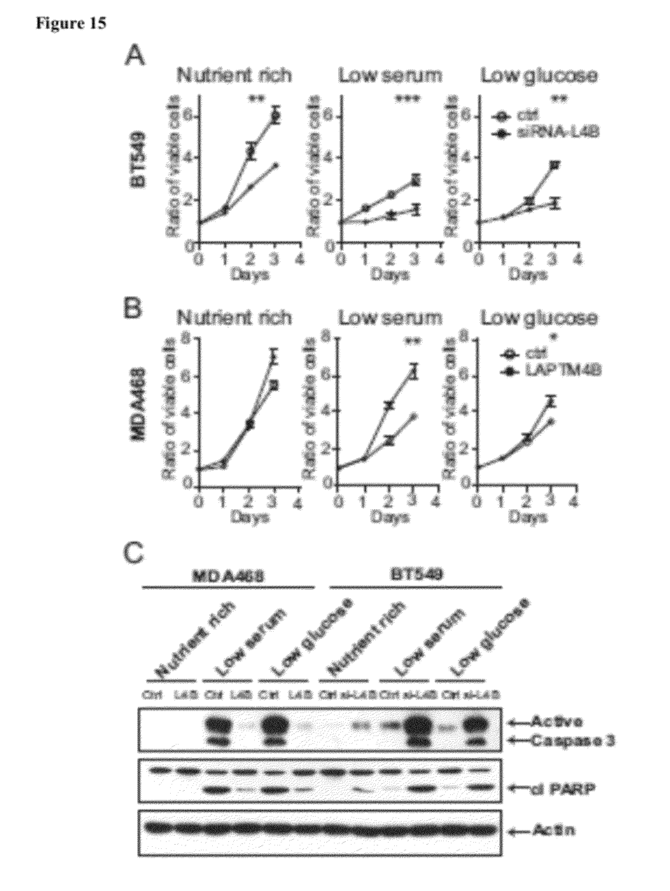

The previous experiments indicate that overexpression of LAPTM4B results in decreased sensitivity of tumor cells to insults that trigger LMP, lysosome-mediated programmed cell death, or induce autophagy. Therefore, the effect of modulating LAPTM4B expression levels on cell survival in cells exposed to various environmental stressors was examined. In BT549 cells which have amplification and over-expression of LAPTM4B, siRNA depletion of LAPTM4B resulted in approximately 50% decrease (P=0.0267) in viable cells when cells were cultured in nutrient-rich medium. Depletion of LAPTM4B resulted in a similar or more pronounced decrease in viable cells when grown in low serum or low glucose medium (P=0.001 and 0.0260, respectively; FIG. 15A). MDA468 breast cancer cells have inherently low normal levels of LAPTM4B expression. MDA468 cells with forced over-expression of LAPTM4B had similar cell viability to parental cells when cultured in nutrient-rich media (P=0.105). These results indicate that a certain level of LAPTM4B may be required for tumor cell survival, but its over-expression does not directly promote in vitro cell growth under normal culture conditions. However, the cells with over-expression of LAPTM4B showed higher cell viability than control cells when cultured in low serum or low glucose medium (P=0.0256 and 0.0960, respectively; FIG. 15B). Cell lysates from cells grown in nutrient-rich medium, low serum or glucose free medium were examined for markers of caspase activation and apoptosis. As shown in FIG. 15C, the decreased cell viability of BT549 cells after knockdown of LAPTM4B was associated with increased activation of caspase 3 and PARP cleavage. Similarly, the enhanced survival of MDA468 cells over-expressing LAPTM4B in nutrient deprived conditions was associated with much lower levels of active caspase 3 and PARP cleavage. These results support our hypothesis that high expression of LAPTM4B potentiates the growth and survival of breast cancer cells under metabolic stress.

It was next determined whether modulation of LAPTM4B affects survival of cells exposed to other types of stress. As shown in FIGS. 13A-13B, expression levels of LAPTM4B had no effect on cell viability when cells were cultured in acidified medium. Exposure to hypoxic stress showed variable results. Forced overexpression of LAPTM4B in MDA468 cells had no significant effect on cell viability of cells grown in low oxygen (FIG. 13C). However, BT549 with LAPTM4B-depletion were more sensitive to hypoxia and had reduced viability compared to parental BT549 cells that over-express LAPTM4B (FIG. 13D).

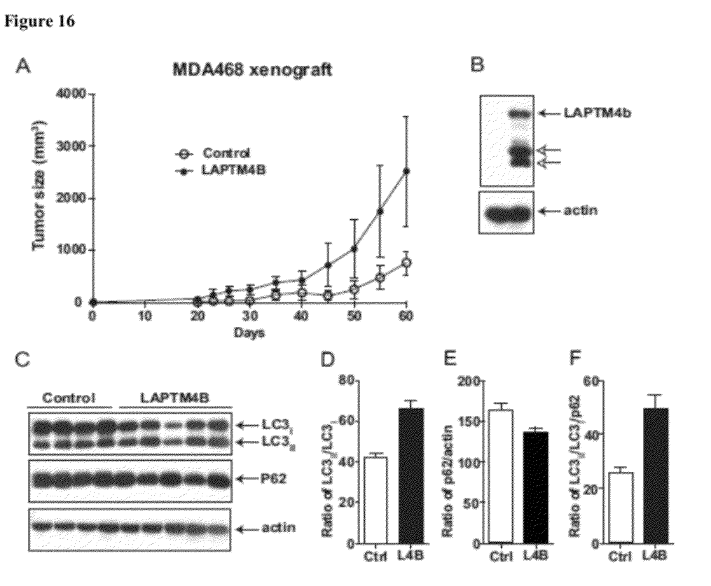

The effects of LAPTM4B modulation on tumor growth and survival in an in vivo xenograft model were also examined. MDA468 cells with stable expression of either a control vector or a vector driving LAPTM4B over-expression were inoculated into the cleared mammary fat pad of nude mice. After two months, it was found that the tumor xenografts that over-expressed LAPTM4B grew much faster than control xenografts (t-test P=0.033, FIG. 16A). Examination of the xenograft tumor explants demonstrated that each continued to over-express LAPTM4B compared to control xenograft tumors (FIG. 16B); the presence of the smaller proteolytic fragments indicates proper delivery of LAPTM4B to the lysosomes (Vergarajauregui et al. (2010) J. Cell Sci., 124, 459-468). We examined tumor lysates for markers of autophagy by immunoblot (FIG. 16C). LAPTM4B-overexpressing xenografts demonstrated higher LC3_{II}/LC3_{I} ratio (P=0.0040, FIG. 16D), decreased p62 levels (P=0.0363, FIG. 16E), and higher LC3_{II}/LC3_{I}/p62 ratio (P=0.0074, FIG. 16F) consistent with increased autophagic flux. This result indicates that LAPTM4B over-expression promotes tumor growth in vivo, perhaps by promoting greater tolerance to stress through increased induction of autophagy.

The cancer gene LAPTM4B is one of two genes amplified and overexpressed from chromosome 8q22 which predicts for de novo anthracycline resistance and metastatic recurrence in breast cancer patients (Li et al. (2010) Nat. Med., 16, 214-218). One mechanism by which LAPTM4B promotes chemotherapy resistance is by retention of anthracyclines in a cytoplasmic compartment thereby preventing nuclear drug localization and drug-induced DNA damage. The frequent occurrence of 8q amplification and overexpression of LAPTM4B in treatment-naive cancers raised the possibility that LAPTM4B may provide a growth or survival advantage to tumor cells in the absence of a therapy challenge, resulting in its selection and upregulation in primary (untreated) cancers. The results provided herein indicate that LAPTM4B is localized in lysosomes of mammary cells and promotes lysosome membrane stability. Decreased expression of LAPTM4B leads to increased LMP, lysosomal release of cathepsins and lysosome-mediated cell death. This effect is more pronounced when cells are exposed to anthracycline chemotherapy, consistent with the increased sensitivity to anthracyclines in tumors with low LAPTM4B expression (Li et al. (2010) Nat. Med., 16, 214-218). LAPTM4B also plays a role in small molecule trafficking, including drugs, from lysosome to other cellular compartments, as evidenced by the reduction of doxorubicin trafficking from the lysosome compartment to the nucleus when LAPTM4B is overexpressed. Thus, it was determined herein that LAPTM4B plays a critical role in autophagy and insufficient levels of LAPTM4B results in a failure of autophagosome-lysosome fusion causing a block in autolysosome formation and decreased autophagy flux. In contrast, overexpression of LAPTM4B in breast cancer cells that have normal DNA copy number of 8q22 and express normal levels of LAPTM4B results in increased autophagy flux. These observations indicate that the proper function of LAPTM4B is required for lysosome-mediated autophagy maturation. A recent study showed that transient over-expression of LAPTM4B without coordinate increase of the expression of its partner MCOLN1 in retinal epithelial cells led to increased LC3_{II} levels due to an accumulation of autophagosomes (Vergarajauregui et al. (2010) J. Cell Sci., 124, 459-468). This finding could result from increased autophagy induction or a block in autophagy flux, but is consistent with the notion that appropriate levels of LAPTM4B are crucial to proper lysosome-mediated autophagy.

Fusion of lysosome with autophagosome is a critical step for autophagy maturation. Rab7, SKD1, Lamp2, UVRAG and Rebicon have been shown to be essential for this process (Burman et al. (2010) Semin. Immunopathol., 32, 397-413; Eskelinen, E. L. (2005) Autophagy, 1, 1-10; Mehrpour et al. (2010) Cell Res., 20, 748-762; Vergarajauregui et al. (2010) J. Cell Sci., 124, 459-468; Liang et al. (2008) Nat. Cell. Biol., 10, 776-787; Matsunaga et al. (2009) Nat. Cell Biol., 11, 385-396). Frequent frameshift mutations have been identified in the UVRAG gene in colon and gastric cancers (Kim et al. (2008) Hum. Pathol., 39, 1059-1063); however, no significant aberrations in autophagy were found in cancer cells carrying this mutation (Knaevelsrud et al. (2010) Autophagy, 6, 863-870). LAPTM4B thus represents the first gene crucial for autophagy maturation and flux that is amplified in cancer (Li et al. (2010) Nat. Med., 16, 214-218; Hu et al. (2009) Cancer Cell, 15, 9-20) and is associated with treatment resistance and poor clinical outcome in various cancers (Shao et al. (2003) Oncogene, 22, 5060-5069; Li et al. (2010) Nat. Med., 16, 214-218; Yang et al., (2010) J. Cancer Res. Clin. Oncol., 136, 275-281; Meng et al. (2010), Int. J. Gynecol. Cancer, 20, 745-750; Yang et al. (2010) Cancer Lett., 294, 236-244; Hu et al. (2009) Cancer Cell, 15, 9-20). Metadherin was recently shown to induce autophagy in different cell lineages by increasing expression of atg5 and activating AMP kinase (Bhutia et al. (2010) Proc. Natl. Acad. Sci. USA, 107, 22243-22248). Interestingly, the gene for metadherin, MTDH, is the immediate neighbor to LAPTM4B on chromosome 8q22 and is co-amplified with LAPTM4B in cancers. It is thus proposed herein that the coordinate amplification of the two genes may activate simultaneously the two major stages of the autophagy pathway and thereby enhance tumor survival.

Tumors frequently experience elevated metabolic stress from nutrient and oxygen deprivation due to insufficient angiogenesis (Jin et al. (2007) Autophagy, 3, 28-31). The high metabolic demands of cell proliferation and altered metabolism provide additional tumor cell-intrinsic stress. Thus, tumors may be more dependent on survival mechanisms such as autophagy to maintain growth (White et al. (2009) Clin. Cancer Res., 15, 5308-5316). It is shown herein that the roles of LAPTM4B in limiting lysosome-mediated cell death and promoting autophagy have significant survival effects in cancer cells, including greater resistant to nutrient deprivation, hypoxia, and chemotherapy-induced genotoxic stress. It is further demonstrated herein that LAPTM4B pro-survival effect is associated with greater in vivo tumor growth. The new knowledge from this study contributes to a better understanding of the molecular mechanisms of lysosome-mediated drug resistance and the particular dependency of tumors on lysosomal function for cell survival. As normal tissues are seldom under nutrient deficiency, there may be a significant therapeutic window for inhibiting LAPTM4B in tumors that over-express this gene and are dependent on it for survival. Targeting LAPTM4B to inhibit tolerance to stress can serve as a new strategy for anticancer drugs and provide a way to clip the Achilles heel of many cancers.

TABLE 1
Pam Probe List

| | | | | | | | Expression | | Expression | |
|---|---|---|---|---|---|---|---|---|---|---|
| | | | | | | | and FISH | | and SNP copy | |
| | | | | | | Expression | 8q22 copy | | Correlation | |
| | | | | | | &recurrence | Kendall | | Kendall | |
| | Gene | | | | | PAM | | p- | | p- |
| Probe | ID | Gene | Gene Name | Chrom. | Start | score | Correlation | value | Correlation | value |
| 206165_s_at | 9635 | CLCA2 | | chr 1 | 86601845 | 0.2186 | | | | |
| 206164_at | 9635 | CLCA2 | | chr 1 | 86601845 | 0.2751 | | | | |
| 206166_s_at | 9635 | CLCA2 | | chr 1 | 86601845 | 0.2591 | | | | |
| 217528_at | 9635 | CLCA2 | | chr 1 | 86633528 | 0.2389 | | | | |
| 213048_s_at | NA | NA | | chr 1 | 92251249 | 0.0263 | | | | |
| 214370_at | 6279 | S100A8 | | chr 1 | 150175580 | 0.0266 | | | | |
| 202917_s_at | 6279 | S100A8 | | chr 1 | 150175581 | 0.0497 | | | | |
| 223126_s_at | 81563 | C1orf21 | | chr 1 | 181087866 | −0.0243 | | | | |
| 235599_at | 339535 | LOC339535 | | chr 1 | 234969703 | 0.1972 | | | | |
| 204914_s_at | 6664 | SOX11 | | chr 2 | 5783376 | 0.0449 | | | | |
| 204915_s_at | 6664 | SOX11 | | chr 2 | 5783396 | 0.1111 | | | | |
| 201287_s_at | 6382 | SDC1 | | chr 2 | 20322885 | 0.2254 | | | | |
| 212777_at | 6654 | SOS1 | | chr 2 | 39122573 | 0.0186 | | | | |
| 208724_s_at | 5861 | RAB1A | | chr 2 | 65226664 | 0.0392 | | | | |
| 224311_s_at | 51719 | CAB39 | | chr 2 | 231450221 | 0.0079 | | | | |
| 204992_s_at | 5217 | PFN2 | | chr 3 | 151165391 | 0.0325 | | | | |
| 211812_s_at | 8706 | B3GALNT1 | | chr 3 | 162286248 | 0.0099 | | | | |
| 212592_at | 10117 | ENAM | | chr 4 | 71886293 | −0.0461 | | | | |
| 220225_at | 50805 | IRX4 | | chr 5 | 1930548 | 0.0034 | | | | |
| 229185_at | 3977 | LIFR | | chr 5 | 38592696 | 0.0495 | | | | |
| 212195_at | 3572 | IL6ST | | chr 5 | 55268230 | −0.0152 | | | | |
| 210571_s_at | 8418 | CMAH | | chr 6 | 25189776 | 0.1079 | | | | |
| 1557910_at | 3326 | HSP90AB1 | | chr 6 | 44322809 | 0.0052 | | | | |
| 214359_s_at | 3326 | HSP90AB1 | | chr 6 | 44327296 | 0.0094 | | | | |
| 214581_x_at | 27242 | TNFRSF21 | | chr 6 | 47307891 | 0.0427 | | | | |
| 208651_x_at | 934 | CD24 | | chr 6 | 111694211 | 0.1329 | | | | |
| 216379_x_at | 934 | CD24 | | chr 6 | 111694211 | 0.1464 | | | | |
| 208650_s_at | 934 | CD24 | | chr 6 | 111694211 | 0.0782 | | | | |
| 209771_x_at | 934 | CD24 | | chr 6 | 111694211 | 0.1598 | | | | |
| 266_s_at | 934 | CD24 | | chr 6 | 111694211 | 0.0923 | | | | |
| 225002_s_at | 25870 | SUMF2 | | chr 7 | 55906221 | −0.0102 | | | | |
| 225945_at | 79027 | ZNF655 | | chr 7 | 98816406 | −0.0646 | | | | |
| 204718_at | 2051 | EPHB6 | | chr 7 | 142069659 | 0.0507 | | | | |
| 218541_s_at | 56892 | C8orf4 | | chr 8 | 40130159 | −0.1438 | −0.305 | 0.00016518 | <0.3 | |
| 225534_at | 51125 | GOLGA7 | | chr 8 | 42515874 | −0.0647 | 0.059 | 0.46857363 | <0.3 | |
| 1552378_s_at | 157506 | RDH10 | | chr 8 | 74369818 | 0.0543 | 0.203 | 0.01213494 | <0.3 | |
| 219121_s_at | 54845 | ESRP1 | epithelial | chr 8 | 95727619 | 0.1002 | 0.350 | 1.49E−05 | 0.411 | 0.00131 |
| | | | splicing | | | | | | | |
| | | | regulatory | | | | | | | |
| | | | protein 1 | | | | | | | |
| 218905_at | 55656 | INTS8 | integrator | chr 8 | 95930909 | 0.0825 | 0.350 | 1.56E−05 | 0.454 | 0.00040 |
| | | | complex | | | | | | | |
| | | | subunit 8 | | | | | | | |
| 205034_at | 9134 | CCNE2 | cyclin E2 | chr 8 | 95961644 | 0.0706 | 0.465 | 9.40E−09 | 0.321 | 0.01230 |
| 211814_s_at | 9134 | CCNE2 | cyclin E2 | chr 8 | 95963035 | 0.1388 | 0.426 | 1.41E−07 | <0.3 | |
| 222699_s_at | 79666 | PLEKHF2 | pleckstrin | chr 8 | 96215227 | 0.0898 | 0.256 | 0.00154424 | 0.436 | 0.00068 |
| | | | homology | | | | | | | |
| | | | domain | | | | | | | |
| | | | containing, | | | | | | | |
| | | | family F | | | | | | | |
| | | | (with FYVE | | | | | | | |
| | | | domain) | | | | | | | |
| | | | member 2 | | | | | | | |
| 212251_at | 92140 | MTDH | metadherin | chr 8 | 98725684 | 0.0174 | 0.494 | 1.04E−09 | 0.461 | 0.00034 |
| 208767_s_at | 55353 | LAPTM4B | lysosomal | chr 8 | 98857236 | 0.0188 | 0.496 | 9.20E−10 | 0.335 | 0.00918 |
| | | | protein | | | | | | | |
| | | | transmembrane | | | | | | | |
| | | | 4 beta | | | | | | | |
| 1554679_a_at | 55353 | LAPTM4B | lysosomal | chr 8 | 98857413 | 0.0319 | 0.451 | 2.55E−08 | 0.327 | 0.01084 |
| | | | protein | | | | | | | |
| | | | transmembrane | | | | | | | |
| | | | 4 beta | | | | | | | |
| 200062_s_at | 6156 | RPL30 | ribosomal | chr 8 | 99124047 | 0.0618 | 0.468 | 7.50E−09 | 0.472 | 0.00024 |
| | | | protein L30 | | | | | | | |
| 202635_s_at | 5440 | POLR2K | polymerase | chr 8 | 101232073 | 0.0244 | 0.506 | 4.19E−10 | 0.382 | 0.00281 |
| | | | (RNA) II | | | | | | | |
| | | | (DNA | | | | | | | |
| | | | directed) | | | | | | | |
| | | | polypeptide | | | | | | | |
| | | | K, 7.0 kDa | | | | | | | |
| 215823_x_at | 26986 | PABPC1 | poly(A) | chr 8 | 101784319 | 0.0768 | 0.345 | 2.02E−05 | 0.359 | 0.00531 |
| | | | binding | | | | | | | |
| | | | protein, | | | | | | | |
| | | | cytoplasmic 1 | | | | | | | |
| 215157_x_at | 26986 | PABPC1 | poly(A) | chr 8 | 101784558 | 0.1592 | 0.320 | 7.87E−05 | 0.378 | 0.00335 |
| | | | binding | | | | | | | |
| | | | protein, | | | | | | | |
| | | | cytoplasmic 1 | | | | | | | |
| 200640_at | 7534 | YWHAZ | tyrosine 3- | chr 8 | 102000089 | 0.0349 | 0.292 | 0.00031223 | 0.382 | 0.00305 |
| | | | monooxygenase/ | | | | | | | |
| | | | tryptophan | | | | | | | |
| | | | 5- | | | | | | | |
| | | | monooxygenase | | | | | | | |
| | | | activation | | | | | | | |
| | | | protein, zeta | | | | | | | |
| | | | polypeptide | | | | | | | |
| 200638_s_at | 7534 | YWHAZ | tyrosine 3- | chr 8 | 102001097 | 0.0101 | 0.235 | 0.00374108 | <0.3 | |
| | | | monooxygenase/ | | | | | | | |
| | | | tryptophan | | | | | | | |
| | | | 5- | | | | | | | |
| | | | monooxygenase | | | | | | | |
| | | | activation | | | | | | | |
| | | | protein, zeta | | | | | | | |
| | | | polypeptide | | | | | | | |
| 200641_s_at | 7534 | YWHAZ | tyrosine 3- | chr 8 | 102001930 | 0.0176 | 0.212 | 0.00897247 | <0.3 | |
| | | | monooxygenase/ | | | | | | | |
| | | | tryptophan | | | | | | | |
| | | | 5- | | | | | | | |
| | | | monooxygenase | | | | | | | |
| | | | activation | | | | | | | |
| | | | protein, zeta | | | | | | | |
| | | | polypeptide | | | | | | | |
| 235247_at | 79977 | GRHL2 | grainyhead- | chr 8 | 102572595 | 0.0295 | 0.205 | 0.01149533 | 0.370 | 0.00404 |
| | | | like 2 | | | | | | | |
| | | | (Drosophila) | | | | | | | |
| 202874_s_at | 528 | ATP6V1C1 | ATPase, H+ | chr 8 | 104102502 | 0.056 | 0.369 | 5.15E−06 | 0.391 | 0.00247 |
| | | | transporting, | | | | | | | |
| | | | lysosomal | | | | | | | |
| | | | 42 kDa, V1 | | | | | | | |
| | | | subunit C1 | | | | | | | |
| 200607_s_at | 5885 | RAD21 | | chr 8 | 117927353 | 0.0427 | 0.390 | 1.46E−06 | <0.3 | |
| 200632_s_at | 10397 | NDRG1 | | chr 8 | 134318600 | 0.0221 | 0.093 | 0.25244788 | <0.3 | |
| 203222_s_at | 7088 | TLE1 | | chr 9 | 81428652 | 0.0146 | | | | |
| 1554640_at | 114299 | PALM2 | | chr 9 | 109482211 | 0.0507 | | | | |
| 214697_s_at | 9991 | ROD1 | | chr 9 | 112064033 | 0.0544 | | | | |
| 1553530_a_at | 3688 | ITGB1 | | chr | 33230500 | 0.0331 | | | | |
| | | | | 10 | | | | | | |
| 202887_s_at | 54541 | DDIT4 | | chr | 73703687 | 0.0077 | | | | |
| | | | | 10 | | | | | | |
| 203571_s_at | 10974 | C10orf116 | | chr | 88718250 | −0.064 | | | | |
| | | | | 10 | | | | | | |
| 203058_s_at | 9060 | PAPSS2 | | chr | 89409627 | 0.0443 | | | | |
| | | | | 10 | | | | | | |
| 203059_s_at | 9060 | PAPSS2 | | chr | 89409637 | 0.1217 | | | | |
| | | | | 10 | | | | | | |
| 222399_s_at | 56889 | TM9SF3 | | chr | 98270273 | 0.0074 | | | | |
| | | | | 10 | | | | | | |
| 228523_at | 340719 | NANOS1 | | chr | 120779907 | 0.1295 | | | | |
| | | | | 10 | | | | | | |
| 219359_at | 80162 | ATHL1 | | chr | 280681 | −0.0894 | | | | |
| | | | | 11 | | | | | | |
| 211730_s_at | 5441 | POLR2L | | chr | 830206 | −0.0073 | | | | |
| | | | | 11 | | | | | | |
| 212561_at | 23258 | RAB6IP1 | | chr | 9116947 | −0.0289 | | | | |
| | | | | 11 | | | | | | |
| 238710_at | 283284 | IGSF22 | | chr | 18682425 | 0.0082 | | | | |
| | | | | 11 | | | | | | |
| 1554712_a_at | 219970 | GLYATL2 | | chr | 58358117 | 0.5153 | | | | |
| | | | | 11 | | | | | | |
| 222758_s_at | 54972 | TMEM132A | | chr | 60448521 | 0.0048 | | | | |
| | | | | 11 | | | | | | |
| 226226_at | 120224 | TMEM45B | | chr | 129227577 | 0.0241 | | | | |
| | | | | 11 | | | | | | |
| 213240_s_at | 3851 | KRT4 | | chr | 51486599 | 0.0409 | | | | |
| | | | | 12 | | | | | | |
| 200998_s_at | 10970 | CKAP4 | | chr | 105134121 | 0.0187 | | | | |
| | | | | 12 | | | | | | |
| 208113_x_at | 5042 | PABPC3 | | chr | 24568313 | 0.1398 | | | | |
| | | | | 13 | | | | | | |
| 215091_s_at | 2971 | GTF3A | | chr | 26896759 | −0.0314 | | | | |
| | | | | 13 | | | | | | |
| 224734_at | 3146 | HMGB1 | | chr | 29930484 | −0.0383 | | | | |
| | | | | 13 | | | | | | |
| 215096_s_at | 2098 | ESD | | chr | 46243391 | −0.0372 | | | | |
| | | | | 13 | | | | | | |
| 211981_at | 1282 | COL4A1 | | chr | 109599315 | 0.0099 | | | | |
| | | | | 13 | | | | | | |
| 200814_at | 5720 | PSME1 | | chr | 23675219 | −0.1133 | | | | |
| | | | | 14 | | | | | | |
| 218572_at | 29082 | CHMP4A | | chr | 23748662 | −0.0439 | | | | |
| | | | | 14 | | | | | | |
| 218571_s_at | 29082 | CHMP4A | | chr | 23748662 | −0.1102 | | | | |
| | | | | 14 | | | | | | |
| 228763_at | 29082 | CHMP4A | | chr | 23752989 | −0.0602 | | | | |
| | | | | 14 | | | | | | |
| 228764_s_at | 29082 | CHMP4A | | chr | 23752989 | −0.0511 | | | | |
| | | | | 14 | | | | | | |
| 243200_at | NA | NA | | chr | 45245212 | 0.1996 | | | | |
| | | | | 14 | | | | | | |
| 209189_at | 2353 | FOS | | chr | 74815311 | −0.029 | | | | |
| | | | | 14 | | | | | | |
| 205965_at | 10538 | BATF | | chr | 75058536 | −0.0102 | | | | |
| | | | | 14 | | | | | | |
| 202510_s_at | 7127 | TNFAIP2 | | chr | 102662416 | −0.0388 | | | | |
| | | | | 14 | | | | | | |
| 218007_s_at | 51065 | RPS27L | | chr | 61233107 | −0.0336 | | | | |
| | | | | 15 | | | | | | |
| 212070_at | 9289 | GPR56 | | chr | 56220138 | 0.0528 | | | | |
| | | | | 16 | | | | | | |
| 201942_s_at | 1362 | CPD | | chr | 25730088 | 0.1112 | | | | |
| | | | | 17 | | | | | | |
| 201940_at | 1362 | CPD | | chr | 25730099 | 0.0315 | | | | |
| | | | | 17 | | | | | | |
| 201943_s_at | 1362 | CPD | | chr | 25730109 | 0.0903 | | | | |
| | | | | 17 | | | | | | |
| 210930_s_at | 2064 | ERBB2 | | chr | 35110005 | 0.0443 | | | | |
| | | | | 17 | | | | | | |
| 238480_at | 125488 | C18orf17 | | chr | 19969090 | −4.00E−04 | | | | |
| | | | | 18 | | | | | | |
| 239911_at | 9480 | ONECUT2 | | chr | 53300852 | 0.0222 | | | | |
| | | | | 18 | | | | | | |
| 219762 s_at | 25873 | RPL36 | | chr | 5641373 | −0.0351 | | | | |
| | | | | 19 | | | | | | |
| 204949_at | 3385 | ICAM3 | | chr | 10305453 | −0.0285 | | | | |
| | | | | 19 | | | | | | |
| 204735_at | 5141 | PDE4A | | chr | 10424636 | −0.0068 | | | | |
| | | | | 19 | | | | | | |
| 218553_s_at | 79047 | KCTD15 | | chr | 38979606 | 0.0289 | | | | |
| | | | | 19 | | | | | | |
| 211657_at | 4680 | CEACAM6 | | chr | 46951336 | 0.0519 | | | | |
| | | | | 19 | | | | | | |
| 203757_s_at | 4680 | CEACAM6 | | chr | 46951346 | 0.0954 | | | | |
| | | | | 19 | | | | | | |
| 211883_x_at | 634 | CEACAM1 | | chr | 47705097 | 0.0388 | | | | |
| | | | | 19 | | | | | | |
| 208191_x_at | 5672 | PSG4 | | chr | 48388742 | 0.0033 | | | | |
| | | | | 19 | | | | | | |
| 217807_s_at | 29997 | GLTSCR2 | | chr | 52940621 | −0.0289 | | | | |
| | | | | 19 | | | | | | |
| 201021_s_at | 11034 | DSTN | | chr | 17498515 | 0.038 | | | | |
| | | | | 20 | | | | | | |
| 41469_at | 5266 | PI3 | | chr | 43236457 | 0.0804 | | | | |
| | | | | 20 | | | | | | |
| 209591_s_at | 655 | BMP7 | | chr | 55179365 | 0.0738 | | | | |
| | | | | 20 | | | | | | |
| 219505_at | 51816 | CECR1 | | chr | 16036312 | −0.0455 | | | | |
| | | | | 22 | | | | | | |
| 204541_at | 23541 | SEC14L2 | | chr | 29117486 | −0.0318 | | | | |
| | | | | 22 | | | | | | |
| 230316_at | 23541 | SEC14L2 | | chr | 29144461 | −0.0276 | | | | |
| | | | | 22 | | | | | | |
| 214243_s_at | 253190 | SERHL2 | | chr | 41274422 | 0.0209 | | | | |
| | | | | 22 | | | | | | |
| 217284_x_at | 253190 | SERHL2 | | chr | 41274524 | 0.09 | | | | |
| | | | | 22 | | | | | | |
| 217276_x_at | 253190 | SERHL2 | | chr | 41275732 | 0.0829 | | | | |
| | | | | 22 | | | | | | |

TABLE 2
Association of clinical and pathologic
factors to risk of distant recurrence

| | Univariate analysis | | Multivariate analysis | |
|---|---|---|---|---|
| | P-value ^{a} | Hazard ratio | P-value | Hazard ratio |
| 8q22 copy gain ^{b} | 0.00021 | 7.77 | 0.014 | 4.8 |
| Tumor size (cm) | 0.0002 | 2.47 | 0.0029 | 2.4 |
| Lymph node positive | 0.042 | 3.33 | 0.36 | 1.81 |
| Tumor grade | 0.084 | 2.19 | 0.58 | 1.31 |
| Age (years) | 0.23 | 1.95 | 0.41 | 1.7 |
| No adjuvant | 0.6 | 0.73 | 0.17 | 0.35 |
| chemotherapy | | | | |

^{a} Cox proportional hazard regression; significant P-value < 0.05 in bold.
^{b} Mean 8q22 copy number ≧3.5 determined by fluorescence in situ hybridization

TABLE 3
Prediction of pathological complete response to epirubicin

| | | ROC curve analysis | |
|---|---|---|---|
| Cohort of Trial | Gene(s) | AUC | P_value |
| 1. TOP Epirubicin | | | |
| All cases (n = 118) | LAPTM4B | 0.375 | 0.0215 |
| | YWHAZ | 0.297 | 0.00065 |
| | LAPTM4B + YWHAZ | 0.315 | 0.00057 |
| ER⁻HER2⁻ (n = 87) | LAPTM4B | 0.349 | 0.046 |
| | YWHAZ | 0.207 | 0.00001 |
| | LAPTM4B + YWHAZ | 0.241 | 0.00006 |
| ER⁻HER2⁺ (n = 27) | LAPTM4B | 0.341 | 0.035 |
| | YWHAZ | 0.389 | 0.157 |
| | LAPTM4B + YWHAZ | 0.365 | 0.069 |
| 2. Cisplatin | | | |
| Triple negative (n = 24) | LAPTM4B | 0.402 | 0.85 |
| | YWHAZ | 0.75 | 0.135 |
| | LAPTM4B + YWHAZ | 0.675 | 0.309 |

TABLE 4
Tumor sample features

| | | | 8q22 | | | | | | | | | | | | | | | |
|---|---|---|---|---|---|---|---|---|---|---|---|---|---|---|---|---|---|---|
| | | SNP | SNP | 8q22 | Time of | Metastasis | Distant | | | | | | | | | | | |
| Patient | Exp Array File | array | copy | FISH | followup | Free | Recur | | Histology | | | | | Tumor | lymph | | | Hormonal |
| # | Name | name | # | copy # | (mo) | Survival | Y/N | Age | type | Grade | ER | PR | HER-2 | size (cm) | nodes | Chemo | Chemo class | Rx |
| 4 | | 45_T27 | 3 | | | | | 83 | Ductal | III | pos | pos | neg | 4.0 | positive | | | |
| 6 | AR2004021143 | | | 2.1 | 59 | | N | 57 | Ductal | II | pos | pos | neg | 2.1 | negative | AC x 4 | Anthracycline-based | Tam |
| 7 | AR2004021294 | | | 2.7 | 81 | | N | 59 | Lobular | I | pos | pos | neg | 1.0 | negative | none | none | Tam |
| 9 | AR2004021296 | | | 3.0 | 65 | | N | 54 | Ductal | II | pos | pos-low | low pos | 1.5 | negative | none | none | Tam |
| 10 | | | | 3.0 | 35 | | N | 53 | Lobular | II | pos | pos | neg | 1.2 | negative | Unknown | Unknown | Tam |
| 11 | AR2004021134 | | | 1.4 | 70 | | N | 81 | Lobular | I | pos | pos-low | neg | 2.5 | positive | none | none | Tam |
| 12 | AR2004021138 | 25_T38 | 2 | 2.9 | 75 | | N | 48 | Ductal | III | neg | neg | neg | 2.5 | negative | AC x 4 | Anthracycline-based | none |
| 14 | AR2004021125 | | | 2.5 | 87 | | N | 45 | Ductal | III | neg | neg | pos (3+) | 1.5 | negative | AC x 4 | Anthracycline-based | none |
| 21 | AR2004021135 | | | | 83 | | N | 45 | Ductal | III | neg | neg | low pos | 3.0 | positive | AC-taxol | Anthracycline-based | none |
| 34 | | 8_T56 | 3 | | | | | 72 | Ductal | III | neg | neg | neg | 3.1 | negative | | | |
| 35 | AR2004021254 | | | 1.6 | 48 | | N | 37 | Ductal | II | pos | pos | pos (3+) | 1.3 | positive | AC-taxol | Anthracycline-based | Tam |
| 55 | | | | 2.8 | | 38 | Y | 54 | Lobular | II | pos | pos | low pos | 3.5 | positive | AC-taxol | Anthracycline-based | Tam |
| 56 | | 7_T50 | 3 | | | | | 85 | Ductal | III | pos | neg | neg | 1.9 | positive | | | |
| 58 | AR2004021144 | 6_T44 | 3 | 3.0 | 85 | | N | 46 | Ductal | III | pos | pos-low | neg | 1.5 | positive | AC-taxol | Anthracycline-based | Tam |
| 60 | AR2004021129 | | | 2.0 | 88 | | N | 41 | Ductal | I | pos | pos | neg | 3.0 | positive | AC-taxol | Anthracycline-based | Tam |
| 65 | AR2004021117 | | | 1.9 | 83 | | N | 45 | Ductal | II | pos-low | pos-low | pos (3+) | 1.7 | positive | AC- | Anthracycline-based | Tam |
| 72 | | 46_T46 | 3 | 6.2 | 85 | | N | 39 | Ductal | III | neg | neg | pos (3+) | 2.8 | positive | AC-taxol | Anthracycline-based | none |
| 73 | AR2004021107 | | | | 85 | | N | 46 | Mixed | II | pos | pos | neg | 0.9 | negative | AC x 4 | Anthracycline-based | Tam |
| 74 | AR2004021110 | | | 1.3 | 82 | | N | 54 | Ductal | II | pos | pos-low | pos (3+) | 2.2 | negative | AC x 4 | Anthracycline-based | Tam |
| 86 | AR2004021105 | | | | | 6 | Y | 46 | Mixed | II | pos | pos | neg | 2.6 | positive | CMF | Other | Tam |
| 87 | AR2004021293 | | | 1.6 | 81 | | N | 58 | Ductal | I | pos | neg | neg | 0.8 | negative | none | none | Tam |
| 91 | AR2004021278 | | | 2.0 | 79 | | N | 57 | Mixed | I | pos | neg | neg | 1.1 | negative | none | none | Tam |
| 92 | AR2004021101 | | | | 80 | | N | 62 | Lobular | I | pos | pos | neg | 3.5 | negative | AC x 4 | Anthracycline-based | Tam |
| 96 | AR2004021104 | 11_T4 | 3 | 1.8 | 51 | | N | 55 | Ductal | III | pos-low | pos-low | pos (3+) | 1.4 | positive | AC x 4 | Anthracycline-based | Tam |
| 119 | AR2004021147 | | | | 81 | | N | 58 | Mixed | II | pos | pos | neg | 3.5 | positive | AC-taxol | Anthracycline-based | Tam |
| 123 | AR2004021112 | | | | 79 | | N | 84 | Mixed | II | pos | pos | neg | 2.2 | positive | none | none | Tam |
| 126 | AR2004021141 | 5_T41 | 4 | 3.3 | 73 | | N | 57 | Ductal | III | pos | pos | low pos | 1.7 | positive | AC- | Anthracycline-based | Tam |
| 129 | AR2004021102 | | | 2.3 | 78 | | N | 43 | Ductal | III | pos | pos | pos (3+) | 1.0 | negative | Unknown | Unknown | unknown |
| 131 | AR2004021103 | | | | 78 | | N | 54 | Ductal | I | pos | pos | low pos | 1.7 | negative | none | none | Tam |
| 138 | AR2004021130 | 4_T30 | 3 | 2.4 | 80 | | N | 46 | Lobular | III | pos | pos | low pos | 2.3 | positive | AC-taxol | Anthracycline-based | Tam |
| 142 | AR2004021274 | 03_142 | 2 | | 88 | | N | 60 | Ductal | III | pos | pos | neg | 2.2 | positive | AC-taxol | Anthracycline-based | Tam |
| 148 | AR2004021121 | 6_T21 | 4 | 4.9 | | 18 | Y | 53 | Ductal | III | neg | neg | neg | 3.0 | negative | AC x 4 | Anthracycline-based | none |
| 150 | AR2004021288 | | | | 83 | | N | 62 | Lobular | I | pos | pos | neg | 5.0 | negative | none | none | Tam |
| 151 | AR2004021255 | | | | 58 | | N | 55 | Ductal | III | neg | neg | pos (3+) | 2.5 | positive | AC-taxol | Anthracycline-based | none |
| 154 | AR2004021252 | | | | 81 | | N | 62 | Ductal | I | pos | pos | neg | 1.5 | negative | none | none | Tam |
| 156 | AR2004021279 | | | 1.3 | | 15 | Y | 57 | Ductal | II | neg | neg | pos (3+) | 2.9 | positive | AC-taxol | Anthracycline-based | none |
| 157 | AR2004021118 | | | | 85 | | N | 35 | Lobular | II | pos | neg | neg | 1.1 | positive | AC x 4 | Anthracycline-based | Tam |
| 162 | AR2004021123 | | | | 83 | | N | 32 | Ductal | II | pos | pos-low | neg | 1.5 | positive | AC-taxol | Anthracycline-based | Tam |
| 164 | AR2004021145 | 2_T45 | 3 | | 77 | | N | 50 | Ductal | III | neg | neg | low pos | 1.9 | negative | AC x 4 | Anthracycline-based | none |
| 165 | AR2004021139 | | | | 77 | | N | 53 | Lobular | II | pos | neg | neg | 2.5 | negative | AC x 4 | Anthracycline-based | Tam |
| 168 | AR2004021275 | | | 2.1 | 47 | | N | 50 | Ductal | I | pos | pos | neg | 1.3 | positive | AC-taxol | Anthracycline-based | Tam |
| 169 | | 10_T84 | 3 | | | | | 31 | Ductal | III | pos-low | pos-low | pos (3+) | 4.5 | positive | | | |
| 174 | AR2004021290 | | | 1.8 | 79 | | N | 50 | Ductal | I | pos | pos-low | neg | 1.4 | negative | none | none | Tam |
| 175 | AR2004021124 | | | 1.3 | 74 | | N | 40 | Ductal | I | pos | neg | neg | 4.0 | positive | AC-taxol | Anthracycline-based | Tam |
| 177 | AR2004021285 | | | 1.8 | 78 | | N | 46 | Lobular | II | pos | pos | neg | 1.2 | negative | CMF | Other | Tam |
| 178 | AR2004021291 | | | 1.2 | 74 | | N | 74 | Ductal | I | pos | pos | neg | 1.1 | negative | none | none | Tam |
| 180 | AR2004021113 | | | 2.8 | 52 | | N | 43 | Ductal | III | pos | neg | pos (3+) | 4.2 | negative | AC x 4 | Anthracycline-based | Tam |
| 182 | AR2004021253 | | | 1.8 | 57 | | N | 44 | Ductal | III | neg | neg | neg | 2.0 | negative | AC x 4 | Anthracycline-based | none |
| 187 | AR2004021263 | | | 2.1 | 77 | | N | 53 | Ductal | III | neg | neg | pos(3+) | 3.0 | negative | AC x 4 | Anthracycline-based | none |
| 192 | AR2004021292 | 05_192 | 3 | 2.2 | 74 | | N | 57 | Ductal | III | pos | pos | neg | 1.1 | positive | none | none | Tam |
| 196 | AR2004021111 | | | 2.5 | 75 | | N | 49 | Ductal | III | neg | neg | pos (3+) | 1.8 | negative | AC x 4 | Anthracycline-based | none |
| 199 | | 08_M199 | 4 | 2.0 | | 76 | Y | 55 | Ductal | III | pos | pos | neg | 2.5 | positive | AC x 4 | Anthracycline-based | Tam |
| 203 | AR2004021257 | | | 1.2 | | 21 | Y | 64 | Ductal | III | neg | neg | neg | 1.7 | negative | none | none | none |
| 204 | AR2004021131 | | | 1.7 | 78 | | N | 36 | Ductal | II | pos | pos | neg | 1.0 | negative | AC x 4 | Anthracycline-based | Tam |
| 206 | AR2004021272 | 47_T72 | 3 | | | 2 | Y | 38 | Mixed | III | pos-low | pos-low | neg | 4.2 | positive | none | none | none |
| 207 | AR2004021115 | | | 1.5 | 59 | | N | 45 | Lobular | II | pos | pos | neg | 2.3 | negative | none | none | Tam |
| 212 | AR2004021108 | | | 1.9 | 73 | | N | 44 | Ductal | I | pos | pos | neg | 0.8 | negative | none | none | Tam |
| 214 | AR2004021137 | 20_T37 | 6 | 5.5 | | 14 | Y | 53 | Lobular | III | neg | neg | neg | 5.2 | positive | AC x 2 | Anthracycline-based | none |
| 216 | | | | 2.4 | | 66 | Y | 44 | Mixed | II | pos | pos | pos (3+) | 2.9 | positive | AC x 4 | Anthracycline-based | none |
| 219 | AR2004021277 | | | 2.4 | 54 | | N | 53 | Ductal | III | pos | pos-low | pos (3+) | 2.0 | negative | Unknown | Unknown | Tam |
| 221 | AR2004021289 | | | 2.2 | 54 | | N | 58 | Mixed | I | pos | pos | neg | 1.5 | positive | AC-taxol | Anthracycline-based | Tam |
| 222 | AR2004021276 | | | 1.3 | 83 | | N | 37 | Ductal | I | pos | pos | neg | 1.2 | positive | AC-taxol | Anthracycline-based | Tam |
| 224 | AR2004021297 | | | | 83 | | N | 64 | Mixed | I | pos | pos | neg | 1.3 | negative | none | none | Tam |
| 227 | AR2004021281 | 9_T81 | 4 | 3.5 | | 18 | Y | 84 | Ductal | III | pos | pos | neg | 2.5 | positive | none | none | Tam |
| 228 | AR2004021122 | | | 1.2 | 70 | | N | 60 | Ductal | I | pos | pos-low | neg | 2.3 | negative | AC x 4 | Anthracycline-based | Tam |
| 229 | AR2004021295 | | | 1.5 | 78 | | N | 46 | Ductal | II | pos | pos | neg | 2.1 | negative | AC-taxol | Anthracycline-based | Tam |
| 230 | AR2004021273 | 8_T73 | 2 | 1.6 | 78 | | N | 46 | Ductal | III | pos | pos | neg | 3.0 | positive | AC-taxol | Anthracycline-based | Tam |
| 231 | AR2004021286 | | | 1.3 | 81 | | N | 45 | Lobular | I | pos | pos | neg | 1.5 | positive | AC-taxol | Anthracycline-based | Tam |
| 236 | | | | 4.0 | | 50 | Y | 83 | Ductal | III | neg | neg | pos (3+) | 2.2 | positive | none | none | none |
| 237 | AR2004021265 | | | 1.9 | 82 | | N | 66 | Ductal | I | pos-low | pos | neg | 1.1 | positive | AC x 4 | Anthracycline-based | Tam |
| 238 | AR2004021267 | | | 2.6 | 71 | | N | 63 | Ductal | III | pos | pos | pos (3+) | 1.2 | negative | none | none | none |
| 239 | AR2004021132 | | | 1.8 | 73 | | N | 68 | Ductal | III | neg | neg | neg | 1.1 | negative | Unknown | Unknown | unknown |
| 241 | | | | 1.5 | 85 | | N | 43 | Ductal | III | neg | neg | neg | 1.4 | positive | AC-taxol | Anthracycline-based | none |
| 246 | AR2004021126 | | | 1.6 | 86 | | N | 46 | Ductal | I | pos | pos | neg | 1.1 | negative | AC x 4 | Anthracycline-based | none |
| 247 | | | | 2.2 | | 36 | Y | 44 | Mixed | II | pos | pos-low | pos (3+) | 3.1 | positive | AC-taxol | Anthracycline-based | Tam |
| 249 | AR2004021114 | | | 1.3 | 81 | | N | 54 | Ductal | II | neg | neg | pos (3+) | 2.5 | negative | AC x 4 | Anthracycline-based | none |
| 252 | AR2004021271 | | | 1.9 | 55 | | N | 66 | Ductal | I | pos | pos | neg | 1.2 | positive | none | none | Tam |
| 253 | AR2004021133 | | | 2.3 | 56 | | N | 54 | Ductal | III | neg | neg | neg | 1.1 | negative | AC x 4 | Anthracycline-based | none |
| 254 | AR2005110807 | | | | 71 | | N | 54 | Ductal | II | pos | pos | neg | 2.5 | negative | none | none | Tam |
| 260 | AR2005110808 | | | | 48 | | N | 82 | Ductal | II | pos | pos | neg | 2.5 | positive | none | none | Tam |
| 262 | AR2005110809 | | | | 71 | | N | 48 | Mixed | III | pos | pos | neg | 5 | positive | AC- | Anthracycline-based | unknown |
| 274 | AR2005110811 | | | | 71 | | N | 49 | Ductal | III | pos | pos | pos (3+) | 3 | negative | AC x 4 | Anthracycline-based | Tam |
| 281 | AR2005110813 | | | | 74 | | N | 45 | Ductal | II | neg | pos | neg | 1.2 | positive | AC-taxol | Anthracycline-based | none |
| 283 | AR2005110814 | | | | 36 | | N | 47 | Ductal | III | pos | pos | pos (3+) | 4 | positive | Unknown | Unknown | unknown |
| 292 | AR2004020603 | | | | | 24 | Y | 53 | Ductal | III | neg | neg | pos (3+) | 2.3 | positive | AC-taxol | Anthracycline-based | none |
| 300 | AR2005110816 | | | | | 18 | Y | 56 | Lobular | II | pos | pos | neg | 1.6 | negative | AC x 4 | Anthracycline-based | Tam |
| 308 | AR2005110817 | | | | 73 | | N | 45 | Ductal | III | pos | pos | pos (3+) | 1.9 | negative | AC x 4 | Anthracycline-based | Tam |
| 310 | AR2004020604 | | | 3.4 | 38 | | N | 60 | Ductal | III | neg | neg | neg | 0.9 | negative | AC x 4 | Anthracycline-based | none |
| 312 | | 12_C115 | 5 | 5.3 | 29 | | N | 37 | Ductal | III | neg | neg | pos (3+) | 2.5 | negative | Unknown | Unknown | unknown |
| 316 | AR2004020606 | 1_T116 | 3 | | 60 | | N | 65 | Ductal | III | neg | neg | neg | 3.5 | negative | AC x 4 | Anthracycline-based | none |
| 325 | AR2004020607 | 2_T117 | 5 | 8.0 | | 9 | Y | 48 | Ductal | III | neg | neg | pos (3+) | 1.8 | positive | AC- | Anthracycline-based | none |
| 330 | AR2004020608 | 3_T118 | 3 | 4.2 | 62 | | N | 58 | Ductal | III | neg | neg | neg | 2.1 | positive | AC-taxol | Anthracycline-based | none |
| 333 | | 17_T152 | 4 | | | | | 57 | Ductal | III | neg | neg | neg | 1.8 | N/D | | | |
| 343 | AR2004020609 | 14_C119 | 5 | 6.0 | | 16 | Y | 45 | Ductal | III | neg | neg | pos (3+) | 2.5 | positive | AC-taxol | Anthracycline-based | none |
| 362 | AR2004031210 | 06_362 | 3 | 3.2 | 69 | | N | 63 | Ductal | III | pos | pos | neg | 4.5 | positive | CMF | Other | Tam |
| 363 | AR2004031231 | 07_363 | 3 | 2.2 | 67 | | N | 41 | Ductal | III | pos | pos-low | neg | 4.0 | positive | AC x 4 | Anthracycline-based | Tam |
| 365 | AR2004031221 | | | | 53 | | N | 78 | Ductal | III | pos | pos | neg | 2.3 | positive | none | none | Arimidex |
| 373 | | | | 1.7 | 27 | | N | 39 | Lobular | II | pos | pos-low | neg | 3.5 | positive | CAF | Anthracycline-based | Tam |
| 390 | AR2004020610 | | | 3.6 | 64 | | N | 49 | Ductal | III | neg | neg | neg | 1.2 | positive | AC x 4 | Anthracycline-based | none |
| 396 | AR2004020642 | 16_T151 | 3 | 4.6 | 65 | | N | 41 | Ductal | III | pos | pos | pos (3+) | 1.1 | positive | AC x 4 | Anthracycline-based | Tam |
| 397 | AR2004020611 | | | | 67 | | N | 56 | Ductal | III | neg | neg | pos (3+) | 2.1 | negative | AC x 4 | Anthracycline-based | none |
| 400 | AR2004020612 | | | | | 27 | Y | 85 | Ductal | II | neg | neg | pos (3+) | 2.5 | positive | AC x 4 | Anthracycline-based | none |
| 417 | | 20_T123 | 2 | | | | | 42 | Ductal | III | neg | neg | neg | 3.3 | positive | | | |
| 451 | AR2004020615 | 49_T125 | 3 | 4.1 | 62 | | N | 66 | Ductal | III | neg | neg | neg | 2.5 | negative | AC x 4 | Anthracycline-based | none |
| 452 | | 04_452 | 2 | | | | | 56 | Ductal | III | neg | neg | pos (3+) | 8.5 | positive | | | |
| 475 | | 50_T127 | 2 | 1.9 | 63 | | N | 48 | Ductal | III | neg | neg | neg | 2.2 | positive | AC-taxol | Anthracycline-based | none |
| 488 | AR2004020618 | | | 1.4 | 52 | | N | 58 | Ductal | II | neg | neg | neg | 1.8 | negative | Unknown | Unknown | none |
| 489 | AR2004031209 | 3_T161 | 3 | | 59 | | N | 37 | Ductal | II | pos | pos | neg | 6.5 | positive | CAF | Anthracycline-based | Tam |
| 498 | AR2004020619 | 21_T129 | 4 | 4.3 | | 1 | Y | 79 | Ductal | III | neg | neg | neg | 5.0 | negative | none | none | none |
| 511 | | 48_T173 | 3 | | | | | 42 | Ductal | II | pos | pos-low | neg | 5.5 | positive | | | |
| 524 | | 4_T130 | 3 | | | | | 58 | Ductal | III | neg | neg | neg | 4.0 | positive | | | |
| 534 | AR2004020621 | | | 2.2 | 53 | | N | 58 | Ductal | III | neg | neg | neg | 2.1 | negative | Epirubicin, | Anthracycline-based | none |
| 537 | AR2004031211 | | | | 71 | | N | 71 | Lobular | III | pos | pos | pos (3+) | 2.3 | positive | none | none | Tam |
| 538 | AR2004020622 | | | 3.2 | 54 | | N | 48 | Ductal | III | neg | neg | neg | 1.0 | negative | Unknown | Unknown | none |
| 552 | AR2004031208 | | | | 56 | | N | 49 | Mixed | I | pos | pos | neg | 2.0 | pos.micr | CMF | Other | Tam |
| 554 | AR2004020623 | 5_T133 | 2 | 2.0 | 51 | | N | 50 | Ductal | III | neg | neg | neg | 0.9 | negative | AC x 4 | Anthracycline-based | none |
| 558 | AR2004020624 | 6_T134 | 3 | 2.8 | 48 | | N | 40 | Ductal | III | neg | neg | neg | 2.2 | negative | AC x 4 | Anthracycline-based | none |
| 582 | AR2004020625 | | | 2.8 | 52 | | N | 58 | Ductal | III | neg | neg | neg | 1.0 | negative | AC x 4 | Anthracycline-based | none |
| 588 | AR2004020626 | | | | 50 | | N | 63 | Ductal | III | neg | neg | pos (3+) | 3.5 | positive | AC- | Trastuzumab | none |
| 590 | | 22_T137 | 3 | | | | | 33 | Ductal | III | neg | neg | neg | 2.7 | positive | | | |
| 591 | AR2004020628 | | | | 50 | | N | 41 | Ductal | III | neg | neg | neg | 2.7 | negative | AC-taxol | Anthracycline-based | none |
| 598 | AR2004020629 | | | | 40 | | N | 74 | Ductal | II | neg | neg | neg | 2.5 | negative | none | none | none |
| 609 | AR2004020631 | 7_T140 | 3 | 2.1 | 49 | | N | 48 | Ductal | III | neg | neg | neg | 3.0 | negative | AC x 4 | Anthracycline-based | none |
| 612 | AR2004031222 | | | | 53 | | N | 49 | Ductal | III | pos | pos | neg | 5.6 | pos.micr | CAF | Anthracycline-based | Tam |
| 613 | AR2004020632 | 10_T141 | 5 | 5.2 | 53 | | N | 78 | Ductal | III | neg | neg | neg | 1.3 | negative | none | none | none |
| 621 | AR2004031232 | | | | 55 | | N | 32 | Mixed | II | pos | pos | pos (3+) | 4.5 | positive | AC- | Trastuzumab | Tam |
| 636 | | 18_T636 | 2 | | | | | 44 | Ductal | III | neg | neg | neg | 1.7 | positive | | | |
| 643 | AR2004031204 | | | | 51 | | N | 68 | Mixed | III | neg | neg | pos (3+) | 5.5 | positive | AC-taxd | Anthracycline-based | none |
| 651 | AR2004031219 | | | | 53 | | N | 45 | Lobular | II | pos | pos | neg | 4.0 | positive | AC-taxol | Anthracycline-based | Tam |
| 653 | AR2004020633 | | | 4.1 | 50 | | N | 55 | Ductal | III | neg | neg | neg | 1.4 | negative | AC x 4 | Anthracycline-based | none |
| 656 | AR2004031223 | 41_T656 | 3 | | 54 | | N | 42 | Ductal | III | pos | neg | pos (3+) | 4.0 | positive | AC- | Trastuzumab | Tam |
| 664 | AR2004020634 | 8_T143 | 3 | 5.9 | | 35 | Y | 43 | Ductal | III | neg | neg | neg | 1.7 | negative | AC x 4 | Anthracycline-based | none |
| 665 | AR2004020635 | 23_T144 | 3 | 3.0 | 50 | | N | 72 | Ductal | III | neg | neg | neg | 2.3 | negative | none | none | none |
| 666 | AR2004031202 | | | | 48 | | N | 64 | Ductal | III | pos | pos | pos (3+) | 2.1 | negative | AC x 4 | Anthracycline-based | Arimidex |
| 670 | AR2004020636 | 9_T145 | 5 | 4.3 | 36 | | N | 49 | Ductal | III | neg | neg | pos (3+) | 3.4 | positive | AC-taxol | Anthracycline-based | none |
| 691 | | 24_T146 | 3 | 3.7 | 25 | | N | 57 | Ductal | II | neg | neg | neg | 1.5 | negative | Unknown | Unknown | unknown |
| 780 | | 25_T147 | 2 | | | | | 58 | Ductal | III | neg | neg | neg | 2.5 | negative | | | |
| 799 | AR2004020639 | | | 2.7 | 36 | | N | 43 | Ductal | III | neg | neg | neg | 2.8 | negative | CAF | Anthracycline-based | none |
| 828 | AR2004020640 | 26_T149 | 3 | 2.4 | 39 | | N | 56 | Ductal | III | neg | neg | neg | 2.2 | negative | AC- | Anthracycline-based | none |
| 911 | | 19_T911 | 3 | | | | | 50 | Ductal | III | neg | neg | neg | 4.2 | positive | | | |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

LAPTM4B coding nucleic acid sequence (NCBI Accession NM_018407.4) (SEQ ID NO: 1):

| | |
|---|---|
| 1 | atgacgtcac ggactcgggt cacatggcca agtccgcccc gccccctccc cgtccccgcc |
| 61 | gctgcagcgg tcgccttcgg agcgaagggt accgacccgg cagaagctcg gagctctcgg |
| 121 | ggtatcgagg aggcaggccc gcgggcgcac gggcgagcgg gccgggagcc ggagcggcgg |
| 181 | aggagccggc agcagcggcg cggcgggctc caggcgaggc ggtcgacgct cctgaaaact |
| 241 | tgcgcgcgcg ctcgcgccac tgcgcccgga gcgatgaaga tggtcgcgcc ctggacgcgg |
| 301 | ttctactcca acagctgctg cttgtgctgc catgtccgca ccggcaccat cctgctcggc |
| 361 | gtctggtatc tgatcatcaa tgctgtggta ctgttgattt tattgagtgc cctggctgat |
| 421 | ccggatcagt ataacttttc aagttctgaa ctgggaggtg actttgagtt catggatgat |
| 481 | gccaacatgt gcattgccat tgcgatttct cttctcatga tcctgatatg tgctatggct |
| 541 | acttacggag cgtacaagca acgcgcagcc tggatcatcc cattcttctg ttaccagatc |
| 601 | tttgactttg ccctgaacat gttggttgca atcactgtgc ttatttatcc aaactccatt |
| 661 | caggaataca tacggcaact gcctcctaat tttccctaca gagatgatgt catgtcagtg |
| 721 | aatcctacct gtttggtcct tattattctt ctgtttatta gcattatctt gacttttaag |
| 781 | ggttacttga ttagctgtgt ttggaactgc taccgataca tcaatggtag gaactcctct |
| 841 | gatgtcctgg tttatgttac cagcaatgac actacggtgc tgctaccccc gtatgatgat |
| 901 | gccactgtga atggtgctgc caaggagcca ccgccacctt acgtgtctgc ctaa |

LAPTM4B protein sequence (NCBI Accession Number NP_060877.3) (SEQ ID NO: 2):

| | |
|---|---|
| 1 | mtsrtrvtwp spprplpvpa aaavafgakg tdpaearssr gieeagprah gragreperr |
| 61 | rsrqqrrggl qarrstllkt cararatapg amkmvapwtr fysnscclcc hvrtgtillg |
| 121 | vwyliinavv llillsalad pdqynfssse lggdfefmdd anmciaiais llmilicama |
| 181 | tygaykqraa wiipffcyqi fdfalnmlva itvliypnsi qeyirqlppn fpyrddvmsv |
| 241 | nptclvliil lfisiiltfk gyliscvwnc yryingrnss dvlvyvtsnd ttvllppydd |
| 301 | atvngaakep pppyvsa |

| YWHAZ coding nucleic acid sequence (NCBI Accession NM_003406.3) (SEQ ID NO: 3): | |
|---|---|
| 1 | atggataaaa atgagctggt tcagaaggcc aaactggccg agcaggctga gcgatatgat |
| 61 | gacatggcag cctgcatgaa gtctgtaact gagcaaggag ctgaattatc caatgaggag |
| 121 | aggaatcttc tctcagttgc ttataaaaat gttgtaggag cccgtaggtc atcttggagg |
| 181 | gtcgtctcaa gtattgaaca aaagacggaa ggtgctgaga aaaaacagca gatggctcga |
| 241 | gaatacagag agaaaattga gacggagcta agagatatct gcaatgatgt actgtctctt |
| 301 | ttggaaaagt tcttgatccc caatgcttca caagcagaga gcaaagtctt ctatttgaaa |
| 361 | atgaaaggag attactaccg ttacttggct gaggttgccg ctggtgatga caagaaaggg |
| 421 | attgtcgatc agtcacaaca agcataccaa gaagcttttg aaatcagcaa aaaggaaatg |
| 481 | caaccaacac atcctatcag actgggtctg gcccttaact tctctgtgtt ctattatgag |
| 541 | attctgaact ccccagagaa agcctgctct cttgcaaaga cagcttttga tgaagccatt |
| 601 | gctgaacttg atacattaag tgaagagtca tacaaagaca gcacgctaat aatgcaatta |
| 661 | ctgagagaca acttgacatt gtggacatcg gatacccaag gagacgaagc tgaagcagga |
| 721 | gaaggagggg aaaattaa |
| YWHAZ protein sequence (NCBI Accession Number NP_003397.1) (SEQ ID NO: 4): | |
| 1 | mdknelvqka klaeqaeryd dmaacmksvt eqgaelsnee rnllsvaykn vvgarrsswr |
| 61 | vvssieqkte gaekkqqmar eyrekietel rdicndvlsl lekflipnas qaeskvfylk |
| 121 | mkgdyyryla evaagddkkg lvdqsqqayq eafeiskkem qpthpirlgl alnfsvfyye |
| 181 | ilnspekacs laktafdeai aeldtlsees ykdstlimql lrdnitlwts dtqgdeaeag |
| 241 | eggen |

1. A method of predicting the outcome of treatment of a subject with an anthracycline, wherein the subject has a cell hyperproliferative disorder, comprising obtaining a biological sample from the subject, comparing the copy number of a marker in the sample to a control copy number of the marker, wherein said marker comprises region 98,778K to 101,970K on human chromosome 8 or a fragment thereof, and determining therefrom the outcome of treatment of the subject with an anthracycline.

2. (canceled)

3. The method of claim 1, wherein said suitable treatment regimen comprises treatment with an anthracycline when the copy number of the marker is equal to or less than the control copy number of the marker or does not comprise treatment with an anthracycline when the copy number of the marker is greater than the control copy number of the marker.

4. The method of claim 1, wherein the control copy number of the marker is the wild type copy number of the marker in the species to which the subject belongs.

5. A method of predicting the outcome of treatment of a subject with an anthracycline wherein the subject has a hyperproliferative disorder, comprising obtaining a biological sample from the subject, and comparing:
a) the amount, structure, subcellular localization, and/or activity of at least one marker in a subject sample, wherein the marker is selected from the group consisting of LAPTM4B, YWHAZ, and functionally similar homologs thereof; and
b) the amount, structure, subcellular localization, and/or activity of the at least one marker in a control,
wherein a significant difference in the amount, structure, subcellular localization, and/or activity of the at least one marker in the sample and the amount, structure, subcellular localization, and/or activity in the control is predictive of the outcome of treatment of the subject with an anthracycline.

6. The method of claim 5, wherein the marker is LAPTM4B and YWHAZ.

7. The method of claim 1 or 5, wherein the control is determined from a non cell hyperproliferative disorder cell sample from the subject or member of the same species to which the subject belongs.

8. The method of claim 5, wherein the control amount, subcellular localization, structure, and/or activity is the wild type amount, subcellular localization, structure, and/or activity in the species to which the subject belongs.

9. The method of claim 1 or 5, wherein the subject sample is obtained before or after the subject has received adjuvant chemotherapy.

10. (canceled)

11. The method of claim 1 or 5, wherein the sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow.

12. The method of claim 1 or 5, wherein the cell hyperproliferative disorder is selected from the group consisting of breast cancer, ovarian cancer, transitional cell bladder cancer, bronchogenic lung cancer, thyroid cancer, pancreatic cancer, prostate cancer, uterine cancer, testicular cancer, gastric cancer, soft tissue and osteogenic sarcomas, neuroblastoma, Wilms' tumor, malignant lymphoma (Hodgkin's and non-Hodgkin's), acute myeloblastic leukemia, acute lymphoblastic leukemia, Kaposi's sarcoma, Ewing's tumor, refractory multiple myeloma, and squamous cell carcinomas of the head, neck, cervix, and vagina.

13. The method of claim 5, wherein the amount of the marker is determined by determining the level of expression or copy number of the marker.

14. The method of claim 1 or 13, wherein the copy number is determined by using at least one technique selected from the group consisting of fluorescence in situ hybridization (FISH), quantitative PCR (qPCR), comparative genomic hybridization (CGH), and single-nucleotide polymorphism (SNP) array.

15. The method of claim 13, wherein the level of expression of the marker in the sample is assessed by detecting the presence in the sample of a protein corresponding to the marker.

16. The method of claim 15, wherein the protein is detected using a reagent selected from the group consisting of an antibody, an antibody derivative, and an antibody fragment.

17. The method of claim 13, wherein the level of expression of the marker in the sample is assessed by detecting the presence in the sample of a transcribed polynucleotide or portion thereof, wherein the transcribed polynucleotide comprises the marker.

18. The method of claim 17, wherein the transcribed polynucleotide is an mRNA or a cDNA.

19. The method of claim 17, wherein determining the level of expression of the marker comprises the use of at least one technique selected from the group consisting of Northern blot analysis, reverse transcriptase PCR, real-time PCR, RNAse protection, and microarray analysis.

20. The method of claim 17, wherein the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

21. The method of claim 5, wherein the significant difference is an increase in the amount, structure, subcellular localization, and/or activity of the subject sample relative to the control, indicating a reduced likelihood of efficacy of anthracycline treatment of the subject; or wherein the significant difference is a decrease in the amount, structure, subcellular localization, and/or activity of the subject sample relative to the control, indicating an increased likelihood of efficacy of anthracycline treatment of the subject.

22. (canceled)

23. The method of claim 21, wherein the outcome of treatment is measured by at least one criteria selected from the group consisting of survival until mortality, pathological complete response, clinical complete remission, clinical partial remission, clinical stable disease, recurrence-free survival, metastasis free survival, and disease free survival.

24. The method of claim 1 or 5, wherein the anthracycline is selected from the group consisting of mitoxantrone, doxorubicin, aclarubicin, daunorubicin, epirubicin, idarubicin and combinations thereof.

25. A method of treating a subject afflicted with cancer comprising administering to the subject an agent which changes the subcellular localization of or modulates the amount and/or activity of a gene or protein corresponding to at least one marker, wherein the at least one marker is selected from the group consisting of LAPTM4B, YWHAZ, and functionally similar homologs thereof.

26. The method of claim 25, further comprising administering to the subject an anthracycline.

27. The method of claim 26, wherein the anthracycline is selected from the group consisting of mitoxantrone, doxorubicin, aclarubicin, daunorubicin, epirubicin, idarubicin, and combinations thereof.

28-29. (canceled)

30. The method of claim 25, wherein said agent is an antibody or an antigen binding fragment thereof, which specifically binds to a protein corresponding to said marker.

31. The method of claim 30, wherein said antibody is conjugated to a toxin or a chemotherapeutic agent.

32. The method of claim 25, wherein said compound is selected from the group consisting of an RNA interfering agent which inhibits expression of a gene corresponding to said marker; an siRNA molecule or an shRNA molecule which inhibits expression of a gene corresponding to said marker; an antisense oligonucleotide complementary to a gene corresponding to said marker; a peptide or peptidomimetic; a small molecule which inhibits activity of said marker; and an aptamer which inhibits expression or activity of said marker.

33-38. (canceled)

39. A kit for predicting the outcome of treatment of a subject with a cell hyperproliferative disorder with an anthracycline, comprising a reagent for assessing the copy number of at least one marker, wherein the at least one marker comprises region 98,778K to 101,970K on human chromosome 8 or a fragment thereof.

40. A kit for assessing the outcome of treatment of a subject with a cell hyperproliferative disorder with an anthracycline, comprising a reagent for assessing the amount, structure, subcellular localization, and/or activity of at least one marker, wherein the at least one marker is selected from the group consisting of LAPTM4B, YWHAZ, and functionally similar homologs thereof.

41. The kit of claim 39 or 40, wherein the at least one marker is LAPTM4B and YWHAZ.

42. The kit of claim 39 or 40, wherein the reagent is selected from the group consisting of a nucleic acid molecule that hybridizes with the at least one marker.

43. The kit of claim 39 or 40, wherein the reagent is selected from the group consisting of an antibody, and antibody derivative, and an antibody fragment.

44. A kit for treating a subject afflicted with cancer comprising an agent which changes the subcellular localization of or modulates the amount and/or activity of a gene or protein corresponding to at least one marker, wherein the at least one marker is selected from the group consisting of LAPTM4B, YWHAZ, and functionally similar homologs thereof.

45. The kit of claim 44, wherein the at least one marker is LAPTM4B and YWHAZ.

46. The kit of claim 45, further comprising an anthracycline.

47. The kit of claim 46, wherein the anthracycline is selected from the group consisting of mitoxantrone, doxorubicin, aclarubicin, daunorubicin, epirubicin, idarubicin, and combinations thereof.
